SEQUENCE LISTING (2 sequences)

SEQ ID NO: 1 - protein, 108 aa, Artificial Sequence
  Description of Artificial Sequence Synthetic peptide
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 134 aa, Artificial Sequence
  Description of Artificial Sequence Synthetic peptide
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

(19) United States
(12) Patent Application Publication
(10) Pub. No.: US 2007/0269836 A1
(43) Pub. Date: Nov. 22, 2007

(54) Methods and compositions for the diagnosis of venous thromboembolic disease

(76) Inventors: Paul H. McPherson, Encinitas, CA (US); Kenneth F. Buechler, Rancho Santa Fe, CA (US)

Correspondence Address: FOLEY & LARDNER LLP, P.O. BOX 80278, SAN DIEGO, CA, 92138-0278, US

(21) Appl. No.: 11/450,150

(22) Filed: Jun. 9, 2006

Related U.S. Application Data
(60) Provisional application No. 60/688,942, filed on Jun. 9, 2005.

(51) Int. Cl. G01N 33/53; G01N 33/573; G01N 33/68

(52) U.S. Cl. 435/7.4; 435/4; 435/7.1; 435/7.8; 436/183; 436/86

(57) ABSTRACT

The present invention relates to methods and compositions for symptom-based differential diagnosis, prognosis, and determination of treatment regimens in subjects. In particular, the invention relates to methods and compositions selected to rule in or out venous thromboembolic disease, pulmonary embolism, and/or deep vein thrombosis, and for risk stratification in such conditions.

### FIELD OF THE INVENTION

The present invention relates to the identification and use of diagnostic markers related to venous thromboembolic disease (“VTED”). In a various aspects, the invention relates to methods and compositions for use in the diagnosis of VETD, pulmonary embolism, and deep venous thrombosis, and in the stratification of risk in such patients.

### BACKGROUND OF THE INVENTION

The following discussion of the background of the invention is merely provided to aid the reader in understanding the invention and is not admitted to describe or constitute prior art to the present invention.

Venous thromboembolic disease (“VTED”) represents a spectrum of conditions that includes deep venous thrombosis (DVT) and pulmonary embolism (PE). The estimated annual incidence of VETD is 117 cases per 100,000 persons. The incidence rises markedly in persons 60 years and older and may be as high as 900 cases per 100,000 by the age of 85 years. Silverstein et al., Arch. Intern. Med. 158: 585-93, 1998. Risk factors for VETD include increasing age, prolonged immobility, surgery, trauma, malignancy, pregnancy, estrogenic medications (e.g., oral contraceptive pills, hormone therapy, tamoxifen), congestive heart failure, hyperhomocystinemia, diseases that alter blood viscosity (e.g., polycythemia, sickle cell disease, multiple myeloma), and inherited thrombophilias. About 75 percent of patients with VETD have at least one established risk factor. Heit et al., Arch. Intern. Med. 162: 1245-48, 2002.

Most clinically important PEs originate from proximal DVT of the leg, particularly the popliteal, femoral, or iliac veins. Moser and LeMoine, Ann. Intern. Med. 94(4 pt 1): 439-44, 1981. Upper extremity DVT is less common but also may lead to PE. A much less common cause of upper extremity DVT is Paget-Schroetter syndrome (idiopathic upper extremity DVT in young athletes). Classic symptoms of DVT include swelling, pain, and discoloration in the affected extremity. Physical examination may reveal the palpable cord of a thrombosed vein, unilateral edema, warmth, and superficial venous dilation. Such classic signs of DVT are of low predictive value and can occur in other conditions such as musculoskeletal injury, cellulitis, and venous insufficiency. Like DVT, PE is also characterized by a constellation of nonspecific signs and symptoms that are associated with other diseases. The most common symptoms in individuals without preexisting cardiopulmonary disease are dyspnea, pleuritic chest pain, cough, leg edema, leg pain, hemoptysis, and palpitations.

The fibrin degradation polypeptide known as “D-dimer” is a marker of endogenous fibrinolysis and should therefore be detectable in the blood of patients with venous thromboembolic disease. One recent study found that the combination of clinical assessment and a negative D-dimer assay result effectively rules out DVT. Wells et al., N. Engl. J. Med. 349: 1227-1235, 2003. Similarly, use of the D-dimer tests in combination with the clinical assessment is effective in ruling out PE in patients who present to the emergency department. Wells et al., Ann. Intern. Med. 135: 98-107, 2001.

### BRIEF SUMMARY OF THE INVENTION

The present invention relates to the identification and use of markers for the detection of venous thromboembolic disease and in the stratification of risk in VTED patients. The methods and compositions described herein can meet the need in the art for rapid, sensitive and specific diagnostic assay to be used in the diagnosis and differentiation of various forms of VTED. Moreover, the methods and compositions of the present invention can also be used to facilitate the treatment of patients and the development of additional diagnostic and/or prognostic indicators.

In various aspects, the invention relates to materials and procedures for identifying markers that are associated with the diagnosis, prognosis, or differentiation of VTED in a patient; to using such markers in diagnosing and treating a patient and/or to monitor the course of a treatment regimen; to using such markers to identify subjects at risk for one or more adverse outcomes related to VTED; and for screening compounds and pharmaceutical compositions that might provide a benefit in treating or preventing such conditions, e.g., for efficacy.

In a first aspect of the present invention, methods for diagnosing VTED, PE, and/or DVT are described. Such methods comprise performing one or more assays on one or more test samples obtained from the subject, such assay(s) being configured to detect one or more markers as described herein, and using results of the assays performed, typically in the form of a presence or amount of the markers, to assign the presence or absence of VTED, PE, and/or DVT to the subject. A plurality of different assays detecting different biochemical markers are preferably used togetherin a diagnostic “panel.” Such panels may comprise 2, 3, 4, 5, 6, 7, 8, 9, 10, 15, 20, or more or individual assays, and optionally comprise one or more values related to physical characteristics of the subject. These panels most preferably comprise performing a plurality of assays on one or more test samples obtained from the subject, such assays being configured to detect a plurality of markers selected from the group consisting of markers related to blood pressure regulation, markers related to inflammation, markers related to apoptosis, markers related to reactive oxygen species, markers related to myocardial injury, markers related to pulmonary injury, and markers related to coagulation and hemostasis.

In a related aspect, the invention features methods of assigning a risk of one or more clinical outcomes to a subject suffering from VTED, PE, and/or DVT. Such methods comprise performing one or more assays on one or more test samples obtained from the subject, such assay(s) being configured to detect one or more markers as described herein, and using results of the assays performed, typically in the form of a presence or amount of the markers, to assign a risk of one or more clinical outcomes to the subject. As in the case of diagnosis, a plurality of different assays detecting different biochemical markers are preferably used togetherin a prognostic “panel.” Such panels may comprise 2, 3, 4, 5, 6, 7, 8, 9, 10, 15, 20, or more or individual assays, and optionally comprise one or more values related to physical characteristics of the subject.

Suitable markers for use in such methods are described in detail hereinafter. Preferably, such methods comprise performing one or more assays configured to detect one or more markers selected from the group consisting of acidic calponin, adrenomedullin, angiopoietin-4, basic calponin, BMP-4, BNP, BNP₁₋₁₀₈, BNP₃₋₁₀₈, BNP₇₉₋₁₀₈, caspase-3, CCL11, CGRP, CK-BB, CK-MB, CRP, D-dimer, sELAF, endothelin-1, GSTP, hFABP, IL-1ra, IL-25, leptin, sLTBR, MCP-1, MMP-9, MPO, NDKA, neuropilin-2, NGAL, PLGF-1, PLGF 1+2, activated protein C, total protein C, PSAP-A, PSAP-B, PSAP-C, PSAP-D, sRAGE, sPECAM-1, spectrin 145, TIE-2, tissue factor, sTNFR1a, sTNFRSF7, TNFsR14, TpP, UFDP1H, UPA, VCAM-1, VE-cadherin, VEGF, sVEGF-R1, VWF-integrin, and ANP₂₈₋₁₅₁. These preferred markers may be used individually, or in panels that comprise 2, 3, 4, 5, 6, 7, 8, 9, 10, 15, 20, or more or individual assays, and optionally comprise one or more values related to physical characteristics of the subject.

In certain embodiments, result(s) of assay(s) can each be compared to a level (a “threshold”) that is associated with the diagnosis or prognosis of VTED, PE, and/or DVT. By correlating each of the subject's selected marker results to thresholds for each marker of interest, the subject may be assigned to a diagnostic group (e.g., suffering from one of these conditions, or not suffering from one of these conditions. Similarly, by correlating the subject's marker results to prognostic thresholds for each marker, the probability or risk that the subject will suffer one or more future adverse outcomes may be determined.

In other embodiments, particular thresholds for one or more markers in a panel are not relied upon to determine if a profile of marker levels obtained from a subject are indicative of a particular diagnosis or prognosis. Rather, the present invention may utilize an evaluation of the entire profile of markers. For example, by plotting ROC curves for the sensitivity of a particular panel of markers versus 1-(specificity) for the panel at various marker levels, a profile of marker measurements from a subject may be considered together to provide a global probability (a “panel response” expressed either as a numeric score or as a percentage risk) that the symptom(s) observed in an individual are caused by a particular underlying disease. In such embodiments, an increase (or decrease) in a certain subset of markers may be sufficient to indicate a particular diagnosis in one patient, while an increase (or decrease) in a different subset of markers may be sufficient to indicate the same or a different diagnosis in another patient. Methods for performing such analyses are described hereinafter.

In yet other embodiments, multiple determinations of one or more markers can be made, and a temporal change in the markers can be used to rule in or out one or more particular etiologies for observed symptom(s). For example, one or more markers may be determined at an initial time, and again at a second time, and the change (or lack thereof) in the marker level(s) over time determined. In such embodiments, an increase in the marker from the initial time to the second time may be diagnostic of a particular disease underlying one or more symptoms, a particular prognosis, etc. Likewise, a decrease in the marker from the initial time to the second time may be indicative of a particular disease underlying one or more symptoms, a particular prognosis, etc. Temporal changes in one or more markers may also be used together with single time point marker levels to increase the discriminating power of marker panels. In yet another alternative, a “panel response” may be treated as a marker, and temporal changes in the panel response may be indicative of a particular disease underlying one or more symptoms, a particular prognosis, etc.

Receiver Operating Characteristic curves, or “ROC” curves, are typically calculated by plotting the value of a variable versus its relative frequency in “normal” and “disease” populations, where “normal” and “disease” simply indicates the absence and presence of some characteristic of interest. For any particular marker, a distribution of marker levels for subjects with and without a “disease” will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art. See, e.g., Hanley et al., Radiology 143: 29-36 (1982).

Preferably, a plurality of marker assay results are combined to increase the predictive value of the analysis in comparison to that obtained from the markers individually. The skilled artisan will also understand that diagnostic markers, differential diagnostic markers, prognostic markers, time of onset markers, etc., may be combined in a single assay or device. For example, certain marker assay results measured by a device or instrument may be used to diagnose VTED, PE, and/or DVT. Each condition may be diagnosed with sets of markers that may comprise unique markers, or may include markers that overlap with one or both of the other sets. Markers may also be commonly used for multiple purposes by, for example, applying a different set of analysis parameters (e.g., a threshold or a different weighting factor) to the marker(s) for the different purpose(s). For example, a marker at one concentration or weighting may be used, alone or as part of a larger panel, to indicate a diagnosis of VTED, PE, and/or DVT, and the same marker at a different concentration or weighting may be used, alone or as part of a larger panel, to indicate prognosis associated with the diagnosis.

The sensitivity and specificity of a diagnostic and/or prognostic test depends on more than just the analytical “quality” of the test—they also depend on the definition of what constitutes an abnormal result. In practice, In preferred embodiments, markers and/or marker panels are selected to exhibit at least 75% sensitivity, more preferably at least 80% sensitivity, even more preferably at least 85% sensitivity, still more preferably at least 90% sensitivity, and most preferably at least 95% sensitivity, combined with at least 75% specificity, more preferably at least 80% specificity, even more preferably at least 85% specificity, still more preferably at least 90% specificity, and most preferably at least 95% specificity. In particularly preferred embodiments, both the sensitivity and specificity are at least 75%, more preferably at least 80%, even more preferably at least 85%, still more preferably at least 90%, and most preferably at least 95%.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group. In certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

One or more markers may lack predictive value when considered alone, but when used as part of a panel, such markers may be of great value in determining a particular diagnosis/prognosis. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker alone is sufficient to signal a positive result, but only signals a result in combination with one or more other markers in the panel.

While exemplary panels are described herein, assays to detect one or more particular markers may be replaced, added, or subtracted from these exemplary panels while still providing clinically useful results. Panels may comprise both specific markers of VTED, PE, and/or DVT; and/or non-specific markers (e.g., markers that are increased or decreased due to inflammation, regardless of the cause; markers that are increased or decreased due to changes in hemostasis, regardless of the cause, etc.). While non-specific (and/or specific) markers may not individually be diagnostic of VTED, PE, and/or DVT, a particular “fingerprint” pattern of changes may, in effect, act as a specific indicator of a disease or condition. As discussed above, that pattern of changes may be obtained from a single sample, or may optionally consider temporal changes in one or more members of the panel (or temporal changes in a panel response value).

In a particularly preferred embodiment, the plurality of assays used in the diagnostic and prognostic methods and compositions described herein comprises at least one, and preferably two or more, assays configured to detect marker(s) related to coagulation and hemostasis. Particularly preferred markers related to coagulation and hemostasis include those selected from the group consisting of plasmin, thrombin, antithrombin-III, fibrinogen, one or more forms of von Willebrand factor, D-dimer, PAI-1, soluble urokinase plasminogen activator surface receptor (uPAR), Protein C, soluble endothelial protein C receptor (EPCR), TAFI, fibrinopeptide A, plasmin alpha 2 antiplasmin complex, platelet factor 4, platelet-derived growth factor, P-selectin, prothrombin fragment 1+2, B-thromboglobulin, thrombin antithrombin III complex, thrombomodulin, thrombus precursor protein, tissue factor, tissue factor pathway inhibitor-α, and tissue factor pathway inhibitor-β, or markers related thereto. Most preferred markers are selected from the group consisting of D-dimer, uPAR, TpP, and one or more forms of von Willebrand factor, or markers related thereto.

In another particularly preferred embodiment, the plurality of assays used in the diagnostic and prognostic methods and compositions described herein comprises at least one, and preferably two or more, assays configured to detect marker(s) related to blood pressure regulation. Particularly preferred markers related to blood pressure regulation are selected from the group consisting of atrial natriuretic peptide (“ANP), pro-ANP, B-type natriuretic peptide (“BNP”), NT-pro BNP, pro-BNP C-type natriuretic peptide, urotensin II, urocortin I, urocortin II, urocortin III, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, and urodilatin, or markers related thereto. Most preferred markers are selected from the group consisting of BNP, proBNP, NT-proBNP, BNP₇₉₋₁₀₈, and BNP₃₋₁₀₈, or markers related thereto.

In still another particularly preferred embodiment, the plurality of assays used in the diagnostic and prognostic methods and compositions described herein comprises at least one, and preferably two or more, assays configured to detect marker(s) related to inflammation. Particularly preferred markers are selected from the group consisting of acute phase reactants, cell adhesion molecules such as vascular cell adhesion molecule (“VCAM”), intercellular adhesion molecule-1 (“ICAM-1”), intercellular adhesion molecule-2 (“ICAM-2”), and intercellular adhesion molecule-3 (“ICAM-3”), C-reactive protein, HMG-1 (also known as HMGB1), interleukins such as IL-1β, IL-6, IL-8, interleukin-1 receptor agonist, monocyte chemotactic protein-1, caspase-3, lipocalin-type prostaglandin D synthase, mast cell tryptase, eosinophil cationic protein, KL-6, haptoglobin, tumor necrosis factor α, tumor necrosis factor β, Fas ligand, soluble Fas (Apo-1), TRAIL, TWEAK, fibronectin, macrophage migration inhibitory factor (MIF), and vascular endothelial growth factor (“VEGF”), or markers related thereto.

Acute phase reactants may be selected from the group consisting of hepcidin, HSP-60, HSP-65, HSP-70, asymmetric dimethylarginine (an endogenous inhibitor of nitric oxide synthase), matrix metalloproteins 11, 3, and 9, defensin HBD 1, defensin HBD 2, serum amyloid A, oxidized LDL, insulin like growth factor, transforming growth factor β, inter-α-inhibitors, e-selectin, glutathione-S-transferase, hypoxia-inducible factor-1α, inducible nitric oxide synthase (“I-NOS”), intracellular adhesion molecule, lactate dehydrogenase, matrix metalloproteinase-9 (“MMP-9”), monocyte chemoattractant peptide-1 (“MCP-1”), n-acetyl aspartate, prostaglandin E2, receptor activator of nuclear factor (“RANK”) ligand, TNF receptor superfamily member 1A, and cystatin C, or markers related thereto.

Likewise, the plurality of assays used in the diagnostic and prognostic methods and compositions described herein may comprise at least one, and preferably two or more, assays configured to detect marker(s) related to reactive oxygen species. The marker(s) may be selected from the group consisting of superoxide dismutase, glutathione, α-tocopherol, ascorbate, inducible nitric oxide synthase, lipid peroxidation products, nitric oxide, myeloperoxidase, and breath hydrocarbons (preferably ethane), or markers related thereto.

Additional markers and/or marker classes may be added to such panels to provide further ability to discriminate amongst diseases. For example, the inflammatory response and resulting effects on capillaries and reduced oxygenation of tissues implicate one or more markers related to the acute phase response, one or more markers related to vascular tissues, and one or more tissue-specific (e.g., neural-specific) markers, the levels of which are increased in ischemic conditions. Preferably, one or more markers selected from the group consisting of α-2 actin, basic calponin 1, β-1 integrin, acidic calponin, caldesmon, cysteine rich protein-2 (“CRP 2” or “CSRP 2”), elastin, fibrillin 1, latent transforming growth factor beta binding protein 4 (“LTBP 4”), smooth muscle myosin, smooth muscle myosin heavy chain, and transgelin, or markers related thereto (referred to collectively as “markers related to vascular tissue”) may be included in such a panel. Additional marker classes, such as markers related to myocardial injury, markers related to neural tissue injury, markers related to pulmonary injury, etc., are described hereinafter.

Preferred markers related to pulmonary injury may be selected from the group consisting of neutrophil elastase, KL-6, LAMP 3, LAMP3, pulmonary surfactant protein A, pulmonary surfactant protein B, pulmonary surfactant protein C, pulmonary surfactant protein D, phospholipase D, PLA2G5, SFTPC, HT156, and HTII280, or markers related thereto.

Preferred markers related to myocardial injury may be selected from the group consisting of cardiac troponin I (free and/or complexed), cardiac troponin T (free and/or complexed), annexin V, B-enolase, CK-MB, glycogen phosphorylase-BB, heart type fatty acid binding protein, phosphoglyceric acid mutase, and S-100ao, or markers related thereto.

The markers and marker assays described herein may be variously combined to provide suitable marker panels. In various embodiments, the plurality of markers comprises at least one marker related to coagulation and hemostasis and at least one marker related to inflammation; the plurality of markers comprises at least one marker related to blood pressure regulation and at least one marker related to coagulation and hemostasis; the plurality of markers comprises at least one marker related to blood pressure regulation, at least one marker related to inflammation, and at least one marker related to coagulation and hemostasis; the plurality of markers comprises at least one marker related to apoptosis and at least one marker related to coagulation and hemostasis; the plurality of markers comprises at least one marker related to reactive oxygen species and at least one marker related to coagulation and hemostasis; the plurality of markers comprises at least one marker related to myocardial injury and at least one marker related to coagulation and hemostasis; the plurality of markers comprises at least one marker related to pulmonary injury and at least one marker related to coagulation and hemostasis; the plurality of markers comprises at least one marker related to blood pressure regulation, at least one marker related to myocardial injury, and at least one marker related to coagulation and hemostasis; the plurality of markers comprises at least one marker related to blood pressure regulation, at least one marker related to pulmonary injury, and at least one marker related to coagulation and hemostasis; the plurality of markers comprises at least one marker related to blood pressure regulation, at least one marker related to apoptosis, and at least one marker related to coagulation and hemostasis; the plurality of markers comprises at least one marker related to blood pressure regulation, at least one marker related to apoptosis, at least one marker related to inflammation, and at least one marker related to coagulation and hemostasis; or the plurality of markers comprises at least one marker related to blood pressure regulation, at least one marker related to myocardial injury, and at least one marker related to coagulation and hemostasis. These combinations are not meant to be limiting.

These markers may be combined in various combinations. For example, preferred methods may include 1, 2, 3, 4, 5, 6, 7, 8, 9, 10, or more assays configured to detect one or more markers selected from the group consisting of B-type natriuretic peptide, NT-proBNP, proBNP, BNP₇₉₋₁₀₈, BNP₃₋₁₀₈, pulmonary surfactant protein A, B, C, and/or D, caspase-3, CRP, D-dimer, TpP, MCP-1, MMP-9, myeloperoxidase, free cardiac troponin I, complexed cardiac troponin I, free and complexed cardiac troponin I, free cardiac troponin T, complexed cardiac troponin T, free and complexed cardiac troponin T, total cardiac troponin, or marker(s) related thereto.

Particularly preferred panels comprise performing assays configured to detect D-dimer, one or more markers related to blood pressure regulation, preferably selected from the group consisting of B-type natriuretic peptide, NT-proBNP, proBNP, BNP₇₉₋₁₀₈, or BNP₃₋₁₀₈, and/or one or more markers related to myocardial injury, preferably selected from the group consisting of free cardiac troponin I, complexed cardiac troponin I, free and complexed cardiac troponin I, free cardiac troponin T, complexed cardiac troponin T, free and complexed cardiac troponin T, total cardiac troponin, annexin V, B-enolase, CK-MB, glycogen phosphorylase-BB, heart type fatty acid binding protein, phosphoglyceric acid mutase, and S-100ao, or marker(s) related thereto.

Other particularly preferred panels comprise performing assays configured to detect D-dimer, one or more markers related to blood pressure regulation, preferably selected from the group consisting of B-type natriuretic peptide, NT-proBNP, proBNP, BNP₇₉₋₁₀₈, or BNP₃₋₁₀₈, and/or one or more markers related to pulmonary injury, preferably selected from the group consisting of neutrophil elastase, KL-6, LAMP 3, LAMP3, pulmonary surfactant protein A, pulmonary surfactant protein B, pulmonary surfactant protein C, pulmonary surfactant protein D, phospholipase D, PLA2G5, SFTPC, HTI56, and HTII280, or markers related thereto.

Still other particularly preferred panels comprise performing assays configured to detect D-dimer, one or more markers related to blood pressure regulation, preferably selected from the group consisting of B-type natriuretic peptide, NT-proBNP, proBNP, BNP₇₉₋₁₀₈, or BNP₃₋₁₀₈, one or more markers related to myocardial injury, preferably selected from the group consisting of free cardiac troponin I, complexed cardiac troponin I, free and complexed cardiac troponin I, free cardiac troponin T, complexed cardiac troponin T, free and complexed cardiac troponin T, total cardiac troponin, annexin V, B-enolase, CK-MB, glycogen phosphorylase-BB, heart type fatty acid binding protein, phosphoglyceric acid mutase, and S-100ao, and one or more markers related to pulmonary injury, preferably selected from the group consisting of neutrophil elastase, KL-6, LAMP 3, LAMP3, pulmonary surfactant protein A, pulmonary surfactant protein B, pulmonary surfactant protein C, pulmonary surfactant protein D, phospholipase D, PLA2G5, SFTPC, HTI56, and HTII280, or markers related thereto.

As discussed herein, these markers may be measured at a single time point, and/or may be measured at multiple time points for calculation of a change in the marker level(s) over time.

In related aspects, the present invention relates to methods for identifying marker panels for use in the foregoing methods. In developing a panel of markers useful in diagnosis and/or prognosis, data for a number of potential markers may be obtained from a group of subjects by testing for the presence or level of certain markers. The group of subjects may then be divided into sets. For example, a first set includes subjects who have been confirmed as having a disease or, more generally, being in a first condition state. The confirmation of this condition state may be made through a more rigorous and/or expensive testing. A second set of subjects is selected from those who do not fall within the first set.

The data obtained from subjects in these sets includes levels of a plurality of markers. Preferably, data for the same set of markers is available for each patient. Exemplary markers are described herein. Actual known relevance of the marker(s) to the disease of interest is not required. Methods for comparing these subject sets for relevance of one or more markers is described hereinafter. Embodiments of the methods and systems described herein may be used to determine which of the candidate markers are most relevant to the diagnosis of the disease or condition or of a given prognosis.

In another aspect, the invention relates to methods for determining a treatment regimen for use in a subject exhibiting VTED, PE, and/or DVT. The methods preferably comprise performing the methods described herein to rule in or out VTED, PE, and/or DVT; and/or to assign a prognosis to a subject diagnosed with VTED, PE, and/or DVT. One or more treatment regimens can then be selected, based on the condition and/or prognosis assigned to the subject.

In a further aspect, the invention relates to kits to rule in or out VTED, PE, and/or DVT; and or to assign a prognosis to a subject diagnosed with VTED, PE, and/or DVT. These kits preferably comprise devices and reagents for measuring a plurality of marker levels in a patient sample, and instructions for performing the assay. Optionally, the kits may contain one or more means for correlating marker level(s) in order to provide a diagnosis and/or a prognosis. Such kits preferably contain sufficient reagents to perform at least one, and preferably two or more assays described above, and/or Food and Drug Administration (FDA)-approved labeling.

In yet a further aspect, the invention relates to devices to rule in or out VTED, PE, and/or DVT; and/or to assign a prognosis to a subject diagnosed with VTED, PE, and/or DVT. Such devices preferably contain a plurality of diagnostic zones, each of which is configured to provide a signal indicative of one or more of the assay results described above. Such devices may be referred to as “arrays” or “microarrays.” Following reaction of a sample with the devices, a signal is generated from the diagnostic zone(s), which may then be correlated to the presence or amount of the markers of interest. Numerous suitable devices are known to the skilled artisan.

### BRIEF DESCRIPTION OF THE FIGURES

FIGS. 1-4 show box-and-whisker plots for measurements of various subject-derived markers in control and VTED subjects.

### DETAILED DESCRIPTION OF THE INVENTION

The present invention relates in part to methods and compositions for diagnosis, prognosis, and determination of treatment regimens in subjects. In particular, the invention relates to methods and compositions selected to rule in or out VTED, PE, and/or DVT; and or to assign a prognosis to a subject diagnosed with VTED, PE, and/or DVT.

Differential diagnosis refers to methods for diagnosing the particular disease(s) underlying the symptoms in a particular subject, based on a comparison of the characteristic features observable from the subject to the characteristic features of those potential diseases. Depending on the breadth of diseases that must be considered in the differential diagnosis, the types and number of tests that must be ordered by a clinician can be quite large. The clinician must then integrate information obtained from a battery of tests, leading to a clinical diagnosis that best represents the range of symptoms and/or diagnostic test results obtained for the subject.

Patients presenting for medical treatment often exhibit one or a few primary observable changes in bodily characteristics or functions that are indicative of disease. Often, as in the case of VTED, these “symptoms” are nonspecific, in that a number of potential diseases can present the same observable symptom or symptoms.

The present invention describes methods and compositions that can assist in the differential diagnosis of one or more nonspecific symptoms by providing diagnostic markers that are designed to rule in or out one, and preferably a plurality, of possible etiologies for the observed symptoms. Symptom-based differential diagnosis described herein can be achieved using panels of diagnostic markers designed to distinguish between possible diseases that underlie a nonspecific symptom observed in a patient.

Definitions

The term “marker” as used herein refers to proteins, polypeptides, glycoproteins, proteoglycans, lipids, lipoproteins, glycolipids, phospholipids, nucleic acids, carbohydrates, etc. or small molecules to be used as targets for screening test samples obtained from subjects. “Proteins or polypeptides” used as markers in the present invention are contemplated to include any fragments thereof, in particular, immunologically detectable fragments. Markers can also include other measurable physical characteristics including results from blood pressure measurements, temperature measurements, pulse oximetry measurements, patient history, radiography, electrocardiogram, exercise treadmill testing, blood chemistry analysis, echocardiography, bronchoprovocation testing, spirometry, pulse oximetry, esophageal pH monitoring, laryngoscopy, computed tomography, histology, cytology, magnetic resonance imaging, etc. Similarly, markers can also include clinical “scores” such as a pre-test probability assignment, a pulmonary hypertension “Daniel” score, an NIH stroke score, a Sepsis Score of Elebute and Stoner, a Duke Criteria for Infective Endocarditis, a Mannheim Peritonitis Index, an “Apache” score, etc.

Preferably, the methods described hereinafter utilize one or more markers that are derived from the subject. The term “subject-derived marker” as used herein refers to protein, polypeptide, phospholipid, nucleic acid, prion, glycoprotein, proteoglycan, glycolipid, lipid, lipoprotein, carbohydrate, or small molecule markers that are expressed or produced by one or more cells of the subject. The presence, absence, amount, or change in amount of one or more markers may indicate that a particular disease is present, or may indicate that a particular disease is absent. In the case of markers that are known to exist in membrane-bound form, such as type I membrane proteins, cell surface receptors, etc., soluble forms which may be measured in body fluids typically are generated by alternative splicing and/or cleavage and release from the plasma membrane.

Additionally, “non-subject-derived markers” may also be used. Such markers are not derived from the subject as defined herein, but rather that are characteristics of the subject observable by the artisan. Such markers are discussed above, and can include various measureable characteristics and/or clinical scores. This list is not meant to be limiting.

The term “related marker” as used herein refers to one or more fragments of a particular subject-derived marker or its biosynthetic parent that may be detected as a surrogate for the marker itself or as independent markers. For example, human BNP is derived by proteolysis of a 108 amino acid precursor molecule, referred to hereinafter as BNP₁₋₁₀₈. Mature BNP, or “the BNP natriuretic peptide,” or “BNP-32” is a 32 amino acid molecule representing amino acids 77-108 of this precursor, which may be referred to as BNP₇₇₋₁₀₈. The remaining residues 1-76 are referred to hereinafter as BNP₁₋₇₆. Additionally, related markers may be the result of covalent modification of the parent marker, for example by oxidation of methionine residues, ubiquitination, cysteinylation, phosphorylation, nitrosylation, glycosylation, etc.

The sequence of the 108 amino acid BNP precursor pro-BNP (BNP₁₋₁₀₈) is as follows, with mature BNP (BNP₇₇₋₁₀₈) underlined:

(SEQ ID NO: 1)

| | |
|---|---|
| HPLGSPGSAS DLETSGLQEQ RNHLQGKLSE LQVEQTSLEP | 50 |
| LQESPRPTGV | |
| WKSREVATEG IRGHRKMVLY TLRAPRSPKM VQGSGCFGRK | 100 |
| MDRISSSSGL | |
| GCKVLRRH. | 108 |

BNP₁₋₁₀₈ is synthesized as a larger precursor pre-pro-BNP having the following sequence (with the “pre” sequence shown in bold):

(SEQ ID NO: 2)

| | |
|---|---|
| MDPQTAPSRA LLLLLFLHLA FLGGRSHPLG SPGSASDLET | 50 |
| SGLQEQRNHL | |
| QGKLSELQVE QTSLEPLQES PRPTGVWKSR EVATEGIRGH | 100 |
| RKMVLYTLRA | |
| PRSPKMVQGS GCFGRKMDRI SSSSGLGCKV LRRH. | 134 |

While mature BNP itself may be used as a marker in the present invention, the prepro-BNP, BNP₁₋₁₀₈ and BNP₁₋₇₆ molecules represent BNP-related markers that may be measured either as surrogates for mature BNP or as markers in and of themselves. In addition, one or more fragments of these molecules, including BNP-related polypeptides selected from the group consisting of BNP₇₇₋₁₀₆, BNP₇₉₋₁₀₆, BNP₇₆₋₁₀₇, BNP₆₉₋₁₀₈, BNP₇₉₋₁₀₈, BNP₈₀₋₁₀₈, BNP₈₁₋₁₀₈, BNP₈₃₋₁₀₈, BNP₃₉₋₈₆, BNP₅₃₋₈₅, BNP₆₆₋₉₈, BNP₃₀₋₁₀₃, BNP₁₁₋₁₀₇, BNP₉₋₁₀₆, and BNP₃₋₁₀₈ may also be present in circulation. In addition, natriuretic peptide fragments, including BNP fragments, may comprise one or more oxidizable methionines, the oxidation of which to methionine sulfoxide or methionine sulfone produces additional BNP-related markers. See, e.g., U.S. patent Ser. No. 10/419,059, filed Apr. 17, 2003, which is hereby incorporated by reference in its entirety including all tables, figures and claims. Preferred BNP-related molecules are proBNP, NT-proBNP, BNP₇₉₋₁₀₈, and BNP₃₋₁₀₈.

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay. For example, different weighting factors may be applied to the various fragment measurements to provide a more accurate estimate of the amount of natriuretic peptide originally present in the sample.

In a similar fashion, many of the markers described herein are synthesized as larger precursor molecules, which are then processed to provide mature marker; and/or are present in circulation in the form of fragments of the marker. Thus, “related markers” to each of the markers described herein may be identified and used in an analogous fashion to that described for BNP.

Removal of polypeptide markers from the circulation often involves degradation pathways. Moreover, inhibitors of such degradation pathways may hold promise in treatment of certain diseases. See, e.g., Trindade and Rouleau, Heart Fail. Monit. 2: 2-7, 2001. However, the measurement of the polypeptide markers has focused generally upon measurement of the intact form without consideration of the degradation state of the molecules. Assays may be designed with an understanding of the degradation pathways of the polypeptide markers and the products formed during this degradation, in order to accurately measure the biologically active forms of a particular polypeptide marker in a sample. The unintended measurement of both the biologically active polypeptide marker(s) of interest and inactive fragments derived from the markers may result in an overestimation of the concentration of biologically active form(s) in a sample.

The failure to consider the degradation fragments that may be present in a clinical sample may have serious consequences for the accuracy of any diagnostic or prognostic method. Consider for example a simple case, where a sandwich immunoassay is provided for BNP, and a significant amount (e.g., 50%) of the biologically active BNP that had been present has now been degraded into an inactive form. An immunoassay formulated with antibodies that bind a region common to the biologically active BNP and the inactive fragment(s) will overestimate the amount of biologically active BNP present in the sample by 2-fold, potentially resulting in a “false positive” result. Overestimation of the biologically active form(s) present in a sample may also have serious consequences for patient management. Considering the BNP example again, the BNP concentration may be used to determine if therapy is effective (e.g., by monitoring BNP to see if an elevated level is returning to normal upon treatment). The same “false positive” BNP result discussed above may lead the physician to continue, increase, or modify treatment because of the false impression that current therapy is ineffective.

Likewise, it may be necessary to consider the complex state of one or more markers described herein. For example, troponin exists in muscle mainly as a “ternary complex” comprising three troponin polypeptides (T, I and C). But troponin I and troponin T circulate in the blood in forms other than the I/T/C ternery complex. Rather, each of (i) free cardiac-specific troponin I, (ii) binary complexes (e.g., troponin I/C complex), and (iii) ternary complexes all circulate in the blood. Furthermore, the “complex state” of troponin I and T may change over time in a patient, e.g., due to binding of free troponin polypeptides to other circulating troponin polypeptides. Immunoassays that fail to consider the “complex state” of troponin may not detect all of the cardiac-specific isoform of interest.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

As used herein, a “plurality” as used herein refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are “patients,” i.e., living humans that are receiving medical care or are being evaluated in a medical setting. This includes persons with no defined illness who are being investigated for signs of pathology.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, i.e., a marker, the presence, absence, amount, or change in amount of which is indicative of the presence, severity, or absence of the condition.

Similarly, a prognosis is often determined by examining one or more “prognostic indicators.” These are markers, the presence or amount of which in a patient (or a sample obtained from the patient) signal a probability that a given course or outcome will occur. For example, when one or more prognostic indicators reach a sufficiently high (or low) level in samples obtained from such patients, the level may signal that the patient is at an increased probability for experiencing a future outcome in comparison to a similar patient exhibiting a lower (or higher) marker level. A level or a change in level of a prognostic indicator, which in turn is associated with an increased probability of morbidity or death, is referred to as being “associated with an increased predisposition to an adverse outcome” in a patient.

The term “correlating,” as used herein in reference to the use of diagnostic and prognostic markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a profile of marker levels is correlated to a global probability or a particular outcome using ROC curves.

The phrase “determining the diagnosis” as used herein refers to methods by which the skilled artisan can determine the presence or absence of a particular disease in a patient. The term “diagnosis” does not refer to the ability to determine the presence or absence of a particular disease with 100% accuracy, or even that a given course or outcome is more likely to occur than not. Instead, the skilled artisan will understand that the term “diagnosis” refers to an increased probability that a certain disease is present in the subject. In preferred embodiments, a diagnosis indicates about a 5% increased chance that a disease is present, about a 10% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−2%.

The term “discrete” as used herein refers to areas of a surface that are non-contiguous. That is, two areas are discrete from one another if a border that is not part of either area completely surrounds each of the two areas.

The term “independently addressable” as used herein refers to discrete areas of a surface from which a specific signal may be obtained.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3^{rd} Edition, W. E. Paul, ed., Raven Press, N.Y. (1993); Wilson (1994) J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Preferred assays are immunoassays that provide an assay result indicative of polypeptides binding to an antibody, where such antibody specifically binds a target molecule of interest, together with those polypeptides that are related markers and that contain the epitope(s) necessary to bind to the antibody used in the assay. Assays that are “configured to detect” a target molecule of interest may, but need not, specifically detect a particular target (that is, detect detect one form of a molecule (e.g., BNP) but not a related molecule (e.g., proBNP)). Because an antibody epitope is on the order of 8 amino acids, an immunoassay will detect other polypeptides (e.g., related markers) so long as the other polypeptides contain the epitope(s) necessary to bind to the antibody used in the assay. Thus, an assay configured to detect a particular molecule (e.g., detects BNP) may also detect other related polypeptides (e.g., may also detect proBNP, together with one or more fragments of BNP or proBNP) that may exist in the sample, to the extent that such molecules contain the necessary epitopes to be detected in the assay.

The term “specifically binds” is not intended to indicate that an antibody binds exclusively to its intended target and appropriate related markers. Rather, an antibody “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹.

Affinity is calculated as K_{d}=k_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(n−r):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat. No. 6,316,409). The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, is yet even more preferably at least about 1×10⁻⁹ moles/liter, and is most preferably at least about 1×10⁻¹⁰ moles/liter. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

Identification of Marker Panels

In accordance with the present invention, there are provided methods and systems for the identification of one or more markers for differential diagnosis and/or risk stratification of a subject. Suitable methods for identifying markers useful for the diagnosis of disease states are described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No. US03/41453, each of which is hereby incorporated by reference in its entirety, including all tables, figures, and claims.

One skilled in the art will also recognize that univariate analysis of markers can be performed and the data from the univariate analyses of multiple markers can be combined to form panels of markers to differentiate different disease conditions. Such methods include multiple linear regression, determining interaction terms, stepwise regression, neural net methods, etc.

In developing a panel of markers useful in differential diagnosis, data for a number of potential markers may be obtained from a group of subjects by testing for the presence or level of certain markers. The group of subjects is divided into two sets. The first set includes subjects who have been confirmed as having a disease or, more generally, being in a first condition state. For example, this first set of patients may be those diagnosed with VTED, PE, and/or DVT. The confirmation of this condition state may be made through a more rigorous and/or expensive testing to confirm the condition state. Hereinafter, subjects in this first set will be referred to as “diseased.”

The second set of subjects is simply those who do not fall within the first set. Subjects in this second set will hereinafter be referred to as “non-diseased”. Preferably, the first set and the second set each have an approximately equal number of subjects. This set may be normal patients, and/or patients suffering from another cardiovascular disease.

The data obtained from subjects in these sets includes levels of a plurality of markers. Preferably, data for the same set of markers is available for each patient. This set of markers may include all candidate markers that may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve. ROC curves are well known to those skilled in the art.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

As discussed above, the measurement of the level of a single marker may have limited usefulness, e.g., it may be non-specifically increased due to inflammation. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial active region may be initially selected for each marker. The location of the active region may initially be selected at any point, but the selection may affect the optimization process described below. In this regard, selection near a suspected optimal location may facilitate faster convergence of the optimizer. In a preferred method, the active region is initially centered about the center of the overlap region of the two sets of patients. In one embodiment, the active region may simply be a cutoff point. In other embodiments, the active region may have a length of greater than zero. In this regard, the active region may be defined by a center value and a magnitude of length. In practice, the initial selection of the limits of the active region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the active region and another value above the active region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the active region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. In a preferred embodiment, the panel response (R) for each subject (j) is expressed as:
Rⱼ=ΣwᵢI_{i,j},
where i is the marker index, j is the subject index, wᵢ is the weighting coefficient for marker i, I is the indicator value to which the marker level for marker i is mapped for subject j, and Σ is the summation over all candidate markers i. The value “R” may be referred to as a “panel index.”

One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the active region eliminates this concern.

The panel response may also be a general function of several parameters including the marker levels and other factors including, for example, race and gender of the patient, various clinical characteristics, clinical “scores” such as a pulmonary hypertension “Daniel” score, an NIH stroke score, a Sepsis Score of Elebute and Stoner, a Duke Criteria for Infective Endocarditis, a Mannheim Peritonitis Index, an “Apache” score, etc. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the active region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable that may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and active regions for the mapping of marker values to indicators. Certain markers may be then be changed or even eliminated from the panel, and the process repeated until a satisfactory result is obtained. The effective contribution of each marker in the panel may be determined to identify the relative importance of the markers. In one embodiment, the weighting coefficients resulting from the optimization process may be used to determine the relative importance of each marker. The markers with the lowest coefficients may be eliminated or replaced.

In certain cases, the lower weighting coefficients may not be indicative of a low importance. Similarly, a higher weighting coefficient may not be indicative of a high importance. For example, the optimization process may result in a high coefficient if the associated marker is irrelevant to the diagnosis. In this instance, there may not be any advantage that will drive the coefficient lower. Varying this coefficient may not affect the value of the objective function.

To allow a determination of test accuracy, a “gold standard” test criterion may be selected which allows selection of subjects into two or more groups for comparison by the foregoing methods. Measures of test accuracy may be obtained as described in Fischer et al., Intensive Care Med. 29: 1043-51, 2003, and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures:

at least 75% sensitivity, combined with at least 75% specificity;

ROC curve area of at least 0.7, more preferably at least 0.8, even more preferably at least 0.9, and most preferably at least 0.95; and/or

a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

Exemplary Markers

In a preferred embodiment, the following discussion provides Swiss-Prot accession numbers for the human precursor of certain markers described herein. Additional markers are described by their common names hereinafter.

| | |
|---|---|
| Acidic Calponin - Q15417 | PLGF-1 - P49763-2 |
| Adrenomedullin - P35318 | PLGF-2 - P49763-3 |
| Angiopoietin-4 - Q9Y264 | ANP (precursor includes ANP, |
| Basic Calponin - P51911 | proANP and ANP₂₈₋₁₅₁) - P01160 |
| BMP-4 - P12644 | Protein C - P04070 |
| BNP - P16860 (precursor includes | PSAP-A - P07714 |
| BNP, proBNP, BNP₃₋₁₀₈, BNP₇₉₋₁₀₈) | PSAP-B - P07988 |
| CCL11 - P51671 | PSAP-C - P11686 |
| CGRP - P06881 | PSAP-D - P35247 |
| Creatine kinase, B-type - P12277 | RAGE - Q15109 |
| Creatine kinase, M-type - P06732 | sPECAM-1 - P16284 |
| CRP - P02741 | Spectrin 120 - Q13813 |
| Elastin (precursor of soluble elastin | Spectrin 145 - Q13813 |
| fragments) - P15502 | TIE-2 - Q02763 |
| Endothelin-1 - P05305 | Tissue Factor - P13726 |
| GSTP - P09211 | TNFR1a - P19438 |
| hFABP - P05413 | TNFRSFM7 - P26842 |
| IL-1ra - P18510 | TNFsR14 - Q92956 |
| IL-25 - Q8WXB0 | cTNI - P19429 |
| Leptin - P41159 | UFDP1H - Q92890 |
| Lymphotoxin B Receptor - P36941 | UPA - P00749 |
| MCP-1 - P13500 | VCAM-1 - P19320 |
| MMP-9 - P14780 | VE Cadherin - P33151 |
| MPO - Q14862 | VEGF - P15692 |
| MYO - P02144 | VEGF-r1 - P17948 |
| NDKA - P15531 | VEGF-r2 - P35968 |
| Neuropilin-2 - O60462-3 | vWF - P04275 |
| NGAL - P80188 | |

In addition to the use of markers individually (“univariately), a panel consisting of the markers referenced herein may be constructed to provide relevant information related to the differential diagnosis of interest. Such a panel may be constructed using 2, 3, 4, 5, 6, 7, 8, 9, 10, 11, 12, 13, 14, 15, 16, 17, 18, 19 or 20 individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The following provides a brief discussion of additional exemplary markers for use in identifying suitable marker panels by the methods described herein.

Additional Markers

A panel consisting of the markers referenced herein and/or their related markers may be constructed to provide relevant information related to the diagnosis of interest. Such a panel may be constructed using 2, 3, 4, 5, 6, 7, 8, 9, 10, 11, 12, 13, 14, 15, 16, 17, 18, 19, 20, or more individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity.

The following table provides a list of additional preferred markers for use in the present invention. Further detail is provided in US2005/0148029, which is hereby incorporated by reference in its entirety. As described herein, markers related to each of these markers are also encompassed by the present invention. Classification of markers refers to the following categories: Blood pressure regulation: markers related to blood pressure regulation; Inflammation: markers related to inflammation; Apoptosis: markers related to apoptosis; Reactive oxygen: markers related to reactive oxygen species; Myocardial injury: markers related to myocardial injury; Pulmonary injury: markers related to pulmonary injury; Coagulation and hemostasis: markers related to coagulation and hemostasis; Vascular tissue: markers related to vascular tissue injury; Neural tissue injury: markers related to neural tissue injury; Collagen synthesis & degradation: markers related to collagen synthesis and degradation; Tissue injury: markers related to general tissue injury.

| Marker | Classification |
|---|---|
| Myoglobin | Tissue injury |
| E-selectin | Tissue injury |
| VEGF | Tissue injury |
| EG-VEGF | Tissue injury |
| Cardiac Troponin I (free and/or forms | Myocardial injury |
| complexed with other troponin subunits) | |
| Cardiac Troponin T (free and/or forms | Myocardial injury |
| complexed with other troponin subunits) | |
| Annexin V | Myocardial injury |
| B-enolase | Myocardial injury |
| CK-MB | Myocardial injury |
| Glycogen phosphorylase-BB | Myocardial injury |
| Heart type fatty acid binding protein | Myocardial injury |
| Phosphoglyceric acid mutase | Myocardial injury |
| S-100ao | Myocardial injury |
| Kininogen | Blood pressure regulation |
| CGRP II | Blood pressure regulation |
| urotensin II | Blood pressure regulation |
| calcitonin gene related peptide | Blood pressure regulation |
| arg-Vasopressin | Blood pressure regulation |
| Endothelin-1 (and/or Big ET-1) | Blood pressure regulation |
| Endothelin-2 (and/or Big ET-2) | Blood pressure regulation |
| Endothelin-3 (and/or Big ET-3) | Blood pressure regulation |
| procalcitonin | Blood pressure regulation |
| calcyphosine | Blood pressure regulation |
| adrenomedullin | Blood pressure regulation |
| aldosterone | Blood pressure regulation |
| angiotensin 1 (and/or angiotensinogen 1) | Blood pressure regulation |
| angiotensin 2 (and/or angiotensinogen 2) | Blood pressure regulation |
| angiotensin 3 (and/or angiotensinogen 3) | Blood pressure regulation |
| Bradykinin | Blood pressure regulation |
| Tachykinin-3 | Blood pressure regulation |
| calcitonin | Blood pressure regulation |
| Renin | Blood pressure regulation |
| Urodilatin | Blood pressure regulation |
| Ghrelin | Blood pressure regulation |
| Plasmin | Coagulation and hemostasis |
| Thrombin | Coagulation and hemostasis |
| Antithrombin-III | Coagulation and hemostasis |
| Fibrinogen | Coagulation and hemostasis |
| von Willebrand factor | Coagulation and hemostasis |
| D-dimer | Coagulation and hemostasis |
| PAI-1 | Coagulation and hemostasis |
| Protein C | Coagulation and hemostasis |
| Soluble Endothelial Protein C Receptor | Coagulation and hemostasis |
| (EPCR) | |
| TAFI | Coagulation and hemostasis |
| Fibrinopeptide A | Coagulation and hemostasis |
| Plasmin alpha 2 antiplasmin complex | Coagulation and hemostasis |
| Platelet factor 4 | Coagulation and hemostasis |
| Platelet-derived growth factor | Coagulation and hemostasis |
| P-selectin | Coagulation and hemostasis |
| Prothrombin fragment 1 + 2 | Coagulation and hemostasis |
| B-thromboglobulin | Coagulation and hemostasis |
| Thrombin antithrombin III complex | Coagulation and hemostasis |
| Thrombomodulin | Coagulation and hemostasis |
| Thrombus Precursor Protein | Coagulation and hemostasis |
| Tissue factor | Coagulation and hemostasis |
| Tissue factor pathway inhibitor-α | Coagulation and hemostasis |
| Tissue factor pathway inhibitor-β | Coagulation and hemostasis |
| basic calponin 1 | Vascular tissue |
| beta like 1 integrin | Vascular tissue |
| Calponin | Vascular tissue |
| CSRP2 | Vascular tissue |
| elastin | Vascular tissue |
| Endothelial cell-selective adhesion molecule | Vascular tissue |
| (ESAM) | |
| Fibrillin 1 | Vascular tissue |
| Junction Adhesion Molecule-2 | Vascular tissue |
| LTBP4 | Vascular tissue |
| smooth muscle myosin | Vascular tissue |
| transgelin | Vascular tissue |
| Carboxyterminal propeptide of type I | Collagen synthesis & |
| procollagen (PICP) | degradation |
| Collagen carboxyterminal telopeptide (ICTP) | Collagen synthesis & |
| | degradation |
| APRIL (TNF ligand superfamily member 13) | Inflammatory |
| CD27 (TNFRSF7) | Inflammatory |
| Complement C3a | Inflammatory |
| CCL-5 (RANTES) | Inflammatory |
| CCL-8 (MCP-2) | Inflammatory |
| CCL-16 | Inflammatory |
| CCL-19 (macrophage inflammatory | Inflammatory |
| protein-3β) | |
| CCL-20 (MIP-3α) | Inflammatory |
| CCL-23 (MIP-3) | Inflammatory |
| CXCL-5 (small inducible cytokine B5) | Inflammatory |
| CXCL-9 (small inducible cytokine B9) | Inflammatory |
| CXCL-13 (small inducible cytokine B13) | Inflammatory |
| CXCL-16 (small inducible cytokine B16) | Inflammatory |
| DPP-II (dipeptidyl peptidase II) | Inflammatory |
| DPP-IV (dipeptidyl peptidase IV) | Inflammatory |
| Glutathione S Transferase | Inflammatory |
| HIF 1 ALPHA | Inflammatory |
| IL-25 | Inflammatory |
| IL-23 | Inflammatory |
| IL-22 | Inflammatory |
| IL-18 | Inflammatory |
| IL-13 | Inflammatory |
| IL-12 | Inflammatory |
| IL-10 | Inflammatory |
| IL-1-Beta | Inflammatory |
| IL-1ra | Inflammatory |
| IL-4 | Inflammatory |
| IL-6 | Inflammatory |
| IL-8 | Inflammatory |
| Lysophosphatidic acid | Inflammatory |
| MDA-modified LDL | Inflammatory |
| Human neutrophil elastase | Inflammatory |
| C-reactive protein | Inflammatory |
| Insulin-like growth factor | Inflammatory |
| Inducible nitric oxide synthase | Inflammatory |
| Intracellular adhesion molecule | Inflammatory |
| NGAL (Lipocalin-2) | Inflammatory |
| Lactate dehydrogenase | Inflammatory |
| MCP-1 | Inflammatory |
| MMP-1 | Inflammatory |
| MMP-2 | Inflammatory |
| MMP-3 | Inflammatory |
| MMP-7 | Inflammatory |
| MMP-9 | Inflammatory |
| TIMP-1 | Inflammatory |
| TIMP-2 | Inflammatory |
| TIMP-3 | Inflammatory |
| NGAL | Inflammatory |
| n-acetyl aspartate | Inflammatory |
| PTEN | Inflammatory |
| Phospholipase A2 | Inflammatory |
| TNF Receptor Superfamily Member 1A | Inflammatory |
| TNFRSF3 (lymphotoxin β receptor) | Inflammatory |
| Transforming growth factor beta | Inflammatory |
| TREM-1 | Inflammatory |
| TREM-1sv | Inflammatory |
| TL-1 (TNF ligand related molecule-1) | Inflammatory |
| TL-1a | Inflammatory |
| Tumor necrosis factor alpha | Inflammatory |
| Vascular cell adhesion molecule | Inflammatory |
| Vascular endothelial growth factor | Inflammatory |
| cystatin C | Inflammatory |
| substance P | Inflammatory |
| Myeloperoxidase (MPO) | Inflammatory |
| macrophage inhibitory factor | Inflammatory |
| Fibronectin | Inflammatory |
| cardiotrophin 1 | Inflammatory |
| Haptoglobin | Inflammatory |
| PAPPA | Inflammatory |
| s-CD40 ligand | Inflammatory |
| HMG-1 (or HMGB1) | Inflammatory |
| IL-2 | Inflammatory |
| IL-4 | Inflammatory |
| IL-11 | Inflammatory |
| IL-13 | Inflammatory |
| IL-18 | Inflammatory |
| Eosinophil cationic protein | Inflammatory |
| Mast cell tryptase | Inflammatory |
| VCAM | Inflammatory |
| sICAM-1 | Inflammatory |
| TNFα | Inflammatory |
| Osteoprotegerin | Inflammatory |
| Prostaglandin D-synthase | Inflammatory |
| Prostaglandin E2 | Inflammatory |
| RANK ligand | Inflammatory |
| RANK (TNFRSF11A) | Inflammatory |
| HSP-60 | Inflammatory |
| Serum Amyloid A | Inflammatory |
| s-iL 18 receptor | Inflammatory |
| S-iL-1 receptor | Inflammatory |
| s-TNF P55 | Inflammatory |
| s-TNF P75 | Inflammatory |
| sTLR-1 (soluble toll-like receptor-1) | Inflammatory |
| sTLR-2 | Inflammatory |
| sTLR-4 | Inflammatory |
| TGF-beta | Inflammatory |
| MMP-11 | Inflammatory |
| Beta NGF | Inflammatory |
| CD44 | Inflammatory |
| EGF | Inflammatory |
| E-selectin | Inflammatory |
| Fibronectin | Inflammatory |
| RAGE | Inflammatory |
| Neutrophil elastase | Pulmonary injury |
| KL-6 | Pulmonary injury |
| LAMP 3 | Pulmonary injury |
| LAMP3 | Pulmonary injury |
| Lung Surfactant protein A | Pulmonary injury |
| Lung Surfactant protein B | Pulmonary injury |
| Lung Surfactant protein C | Pulmonary injury |
| Lung Surfactant protein D | Pulmonary injury |
| phospholipase D | Pulmonary injury |
| PLA2G5 | Pulmonary injury |
| SFTPC | Pulmonary injury |
| MAPK10 | Neural tissue injury |
| KCNK4 | Neural tissue injury |
| KCNK9 | Neural tissue injury |
| KCNQ5 | Neural tissue injury |
| 14-3-3 | Neural tissue injury |
| 4.1B | Neural tissue injury |
| APO E4-1 | Neural tissue injury |
| myelin basic protein | Neural tissue injury |
| Atrophin 1 | Neural tissue injury |
| Brain derived neurotrophic factor | Neural tissue injury |
| Brain fatty acid binding protein | Neural tissue injury |
| Brain tubulin | Neural tissue injury |
| CACNA1A | Neural tissue injury |
| Calbindin D | Neural tissue injury |
| Calbrain | Neural tissue injury |
| Carbonic anhydrase XI | Neural tissue injury |
| CBLN1 | Neural tissue injury |
| Cerebellin 1 | Neural tissue injury |
| Chimerin 1 | Neural tissue injury |
| Chimerin 2 | Neural tissue injury |
| CHN1 | Neural tissue injury |
| CHN2 | Neural tissue injury |
| Ciliary neurotrophic factor | Neural tissue injury |
| CK-BB | Neural tissue injury |
| CRHR1 | Neural tissue injury |
| C-tau | Neural tissue injury |
| DRPLA | Neural tissue injury |
| GFAP | Neural tissue injury |
| GPM6B | Neural tissue injury |
| GPR7 | Neural tissue injury |
| GPR8 | Neural tissue injury |
| GRIN2C | Neural tissue injury |
| GRM7 | Neural tissue injury |
| HAPIP | Neural tissue injury |
| HIP2 | Neural tissue injury |
| LDH | Neural tissue injury |
| Myelin basic protein | Neural tissue injury |
| NCAM | Neural tissue injury |
| NT-3 | Neural tissue injury |
| NDPKA | Neural tissue injury |
| Neural cell adhesion molecule | Neural tissue injury |
| NEUROD2 | Neural tissue injury |
| Neurofiliment L | Neural tissue injury |
| Neuroglobin | Neural tissue injury |
| neuromodulin | Neural tissue injury |
| Neuron specific enolase | Neural tissue injury |
| Neuropeptide Y | Neural tissue injury |
| Neurotensin | Neural tissue injury |
| Neurotrophin 1,2,3,4 | Neural tissue injury |
| NRG2 | Neural tissue injury |
| PACE4 | Neural tissue injury |
| phosphoglycerate mutase | Neural tissue injury |
| PKC gamma | Neural tissue injury |
| proteolipid protein | Neural tissue injury |
| PTEN | Neural tissue injury |
| PTPRZ1 | Neural tissue injury |
| RGS9 | Neural tissue injury |
| RNA Binding protein Regulatory Subunit | Neural tissue injury |
| S-100β | Neural tissue injury |
| SCA7 | Neural tissue injury |
| secretagogin | Neural tissue injury |
| SLC1A3 | Neural tissue injury |
| SORL1 | Neural tissue injury |
| SREB3 | Neural tissue injury |
| STAC | Neural tissue injury |
| STX1A | Neural tissue injury |
| STXBP1 | Neural tissue injury |
| Syntaxin | Neural tissue injury |
| thrombomodulin | Neural tissue injury |
| transthyretin | Neural tissue injury |
| adenylate kinase-1 | Neural tissue injury |
| BDNF | Neural tissue injury |
| neurokinin A | Neural tissue injury |
| neurokinin B | Neural tissue injury |
| superoxide dismutase | Reactive oxygen |
| glutathione | Reactive oxygen |
| α-tocopherol | Reactive oxygen |
| ascorbate | Reactive oxygen |
| inducible nitric oxide synthase | Reactive oxygen |
| lipid peroxidation products | Reactive oxygen |
| nitric oxide | Reactive oxygen |
| breath hydrocarbons | Reactive oxygen |
| s-acetyl Glutathione | apoptosis |
| cytochrome C | apoptosis |
| Caspase 3 | apoptosis |
| Cathepsin D | apoptosis |
| α-spectrin | apoptosis |

Ubiquitination of Markers

Ubiquitin-mediated degradation of proteins plays an important role in the control of numerous processes, such as the way in which extracellular materials are incorporated into a cell, the movement of biochemical signals from the cell membrane, and the regulation of cellular functions such as transcriptional on-off switches. The ubiquitin system has been implicated in the immune response and development. Ubiquitin is a 76-amino acid polypeptide that is conjugated to proteins targeted for degradation. The ubiquitin-protein conjugate is recognized by a 26S proteolytic complex that splits ubiquitin from the protein, which is subsequently degraded.

It has been reported that sepsis stimulates protein breakdown in skeletal muscle by a nonlysosomal energy-dependent proteolytic pathway, and because muscle levels of ubiquitin mRNA were also increased, the results were interpreted as indicating that sepsis-induced muscle protein breakdown is caused by upregulated activity of the energy-ubiquitin-dependent proteolytic pathway. The same proteolytic pathway has been implicated in muscle breakdown caused by denervation, fasting, acidosis, cancer, and burn injury. Thus, levels of ubiquitinated proteins generally, or of specific ubiquitin-protein conjugates or fragments thereof, can be measured as additional markers of the invention. See, Tiao et al., J. Clin. Invest. 99: 163-168, 1997. Moreover, circulating levels of ubiquitin itself can be a useful marker in the methods described herein. See, e.g., Majetschak et al., Blood 101: 1882-90, 2003.

The skilled artisan will recognize that an assay for ubiquitin may be designed that recognizes ubiquitin itself, ubiquitin-protein conjugates, or both ubiquitin and ubiquitin-protein conjugates. For example, antibodies used in a sandwich immunoassay may be selected so that both the solid phase antibody and the labeled antibody recognize a portion of ubiquitin that is available for binding in both unconjugated ubiquitin and ubiquitin conjugates. Alternatively, an assay specific for ubiquitin conjugates of the muscle protein troponin could use one antibody (on a solid phase or label) that recognizes ubiquitin, and a second antibody (the other of the solid phase or label) that recognizes troponin.

The present invention contemplates measuring ubiquitin conjugates of any marker described herein. Preferred ubiquitin-muscle protein conjugates for detection as markers include, but are not limited to, troponin I-ubiquitin, troponin T-ubiquitin, troponin C-ubiquitin, binary and ternary troponin complex-ubiquitin, actin-ubiquitin, myosin-ubiquitin, tropomyosin-ubiquitin, and α-actinin-ubiquitin.

Assay Measurement Strategies

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwitch, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, and most preferably sandwich immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. Preferred apparatuses perform simultaneous assays of a plurality of markers using a single test device. Particularly useful physical formats comprise surfaces having a plurality of discrete, adressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

Preferred assay devices of the present invention will comprise, for one or more assays, a first antibody conjugated to a solid phase and a second antibody conjugated to a signal development element. Such assay devices are configured to perform a sandwich immunoassay for one or more analytes. These assay devices will preferably further comprise a sample application zone, and a flow path from the sample application zone to a second device region comprising the first antibody conjugated to a solid phase.

Flow of a sample along the flow path may be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Most preferably, sample applied to the sample application zone will contact both a first antibody conjugated to a solid phase and a second antibody conjugated to a signal development element along the flow path (sandwich assay format). Additional elements, such as filters to separate plasma or serum from blood, mixing chambers, etc., may be included as required by the artisan. Exemplary devices are described in Chapter 41, entitled “Near Patient Tests: Triage® Cardiac System,” in The Immunoassay Handbook, 2^{nd} ed., David Wild, ed., Nature Publishing Group, 2001, which is hereby incorporated by reference in its entirety.

A panel consisting of the markers referenced above may be constructed to provide relevant information related to differential diagnosis. Such a panel may be constructed using 1, 2, 3, 4, 5, 6, 7, 8, 9, 10, 15, 20, or more or individual markers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, inpatient, outpatient, physician office, medical clinic, and health screening settings. Furthermore, one skilled in the art can use a single marker or a subset of markers comprising a larger panel of markers in combination with an adjustment of the diagnostic threshold in each of the aforementioned settings to optimize clinical sensitivity and specificity. The clinical sensitivity of an assay is defined as the percentage of those with the disease that the assay correctly predicts, and the specificity of an assay is defined as the percentage of those without the disease that the assay correctly predicts (Tietz Textbook of Clinical Chemistry, 2^{nd} edition, Carl Burtis and Edward Ashwood eds., W.B. Saunders and Company, p. 496).

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In another embodiment, the present invention provides a kit for the analysis of markers. Such a kit preferably comprises devises and reagents for the analysis of at least one test sample and instructions for performing the assay. Optionally the kits may contain one or more means for using information obtained from immunoassays performed for a marker panel to rule in or out certain diagnoses. Other measurement strategies applicable to the methods described herein include chromatography (e.g., HPLC), mass spectrometry, receptor-based assays, and combinations of the foregoing.

Selection of Antibodies

The generation and selection of antibodies may be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60, 1995; Fujiwara et al., Chem. Pharm. Bull. (Tokyo) 44: 1326-31, 1996. The selected polypeptides may then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g., Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

Selecting a Treatment Regimen

Just as the potential causes of any particular nonspecific symptom may be a large and diverse set of conditions, the appropriate treatments for these potential causes may be equally large and diverse. However, once a diagnosis is obtained, the clinician can readily select a treatment regimen that is compatible with the diagnosis. The skilled artisan is aware of appropriate treatments for numerous diseases discussed in relation to the methods of diagnosis described herein. See, e.g., Merck Manual of Diagnosis and Therapy, 17^{th} Ed. Merck Research Laboratories, Whitehouse Station, N.J., 1999.

In addition, since the methods and compositions described herein provide prognostic information, the panels and markers of the present invention may be used to monitor a course of treatment. For example, inproved or worsened prognostic state may indicate that a particular treatment is or is not efficacious.

### EXAMPLES

The following examples serve to illustrate the present invention. These examples are in no way intended to limit the scope of the invention.

### Example 1

### Blood Sampling

Blood specimens are collected by trained study personnel using EDTA as the anticoagulant and centrifuged for greater than or equal to 10 minutes. The plasma component is transferred into a sterile cryovial and frozen at −20° C. or colder. Clinical histories are available for each of the patients to aid in the statistical analysis of the assay data.

### Example 2

### Subject Population

For Examples 6, 7, 8, and 9 below, samples were obtained from 120 normal healthy controls. A study population consisted of prospectively enrolled inpatients and emergency department patients with confirmed pulmonary embolism. The inclusion criterion that triggered a review for eligibility was the presumed diagnosis of pulmonary embolism based upon the initial interpretation of either the computerized tomography (CT) chest angiography scan or ventilation-perfusion (V/Q) lung scan. Exclusion was based upon the following criteria: (1)>12 hours since start of heparin therapy, (2) systolic hypotension (<100 mm Hg for two consecutive measurements obtained greater than 15 min apart, (3) initial treatment with fibrinolytic therapy, catheter fragmentation, or surgical embolectomy, (4) illnesses with a predicted 6-month mortality >50% (e.g., metastatic cancer, end-stage AIDs, end-stage heart or renal failure with no plan for transplantation or hemodialysis therapy), (5) clinical plan to not treat the patient, (6) anatomy or clinical condition that precluded the ability to measure right ventricular function via echocardiography, (7). permanent inability to walk for any reason, (8) prisoners with expected incarceration time >6 months, (9) personal physicians' refusal, (10) inability or unwillingness to participate in the informed consent process. Exclusions that were applied after informed consent were: (1) reinterpretation of CT angiography as negative for pulmonary embolism and no deep venous thrombosis observed on both CT venography and venous ultrasonography, (2) inability to perform echocardiography, (3) patient withdrawal from the study, or loss to follow-up in survivors.

Samples were obtained upon enrollment for 100 subjects diagnosed with PE and/or DVT, and 37 subjects diagnosed with PE and/or DVT and later experiencing an adverse outcome (defined as death, shock, intubation, surgical thrombectomy during hospitalization, or severe disability defined as dilated right ventricle, moderate or severe hypokinesis, sPAP>35 and either rest dyspnea or upon walking <330 m, over 6 months of follow up).

### Example 3

### Biochemical Assays

Individual assays were performed as described below in Examples 4 and 5. Assays were configured to bind the following markers, and results are reported in the following examples using the following units (alternate names and units of measurement are in parenthesis): acidic calponin (ng/mL); adrenomedullin (pg/ml); angiopoietin-4 (ng/ml); basic calponin (ng/ml); bone morphogenetic protein 4 (BMP4) (ng/ml); B-type natriuretic peptide (BNP, BNP₇₇₋₁₀₈) (pg/ml); BNP₁₋₁₀₈ (proBNP) (pg/ml); BNP₃₋₁₀₈ (pg/ml); BNP₇₉₋₁₀₈ (pg/ml); caspase-3 (ng/mL); CCL-11 (pg/ml); calcitonin gene-related peptide (CGRP) (pg/ml); creatine kinase-BB (CK-BB) (ng/ml); creatine kinase-MB (CK-MB) (ng/ml); C-reactive protein (CRP) (μg/ml); D-dimer (ng/ml); soluble elastin fragments (sELAF) (ng/ml); endothelin-1 (pg/ml); glutathione-S-transferase 3 (GSTP) (ng/ml); heart-type fatty acid binding protein (hFABP) (ng/ml); interleukin 1 receptor antagonist (IL-1ra) (pg/ml); interleukin-25 (IL-25) (pg/ml); leptin (ng/ml); soluble lymphotoxin b receptor (sLTBR, sTNFRSF3) (ng/ml); monocyte chemotactic protein-1 (MCP-1) (pg/ml); matrix metalloproteinase-9 (MMP9) (ng/ml); myloperoxidase (MPO) (ng/ml); myoglobin (MYO) (ng/ml); nucleoside diphosphate kinase A (NDKA) (ng/ml); neuropilin-2 (ng/ml); neutrophil gelatinase-associated lipocalin (NGAL) (ng/ml); placental growth factor-1 (PLGF-1) (pg/ml); placental growth factor 1 and 2 (PLGF-1/2) (pg/ml); ANP₂₈₋₁₅₁ (des-Asn-Pro proANP) (ng/ml); activated protein C (ng/ml); total protein C (latent+active PC) (ng/ml); pulmonary surfactant protein A (PSAP-A) (ng/ml); pulmonary surfactant protein B (PSAP-B) (ng/ml); pulmonary surfactant protein C (PSAP-C) (ng/ml); pulmonary surfactant protein D (PSAP-D) (ng/ml); soluble receptor for advanced glycosylation end products (sRAGE) (ng/ml); soluble platelet endothelial cell adhesion molecule-1 (sPECAM-1) (ng/ml); spectrin alpha chain, 120 kda (spectrin 120) (pg/ml); spectrin alpha chain, 145 kda (spectrin 145) (ng/ml); angiopoietin-1 receptor (TIE-2) (ng/ml); tissue factor (pg/ml); soluble tumor necrosis factor receptor 1a (sTNFR1a) (ng/ml); soluble tumor necrosis factor receptor superfamily member 7 (sTNFRSF7, cd27) (ng/ml); soluble tumor necrosis factor receptor superfamily member 14 (TNFsR14) (ng/ml); cardiac troponin I, free and complexed (cTNI) (ng/ml); thrombus precursor protein (TpP) (μg/ml); ubiquitin fusion degradation protein 1 homolog (UFDP1H) (ng/ml); urokinase-type plasminogen activator (UPA) (ng/ml); vascular cell adhesion protein 1 (VCAM-1) (ng/ml); VE-cadherin (cd144 antigen) (ng/ml); vascular endothelial growth factor (VEGF) (pg/ml); soluble flt-1 (sVEGF-R1) (pg/ml); soluble kdr (sVEGF-R2) ng/ml; von Willebrand factor containing a VWF integrin domain (VWF-integrin) (ng/ml).

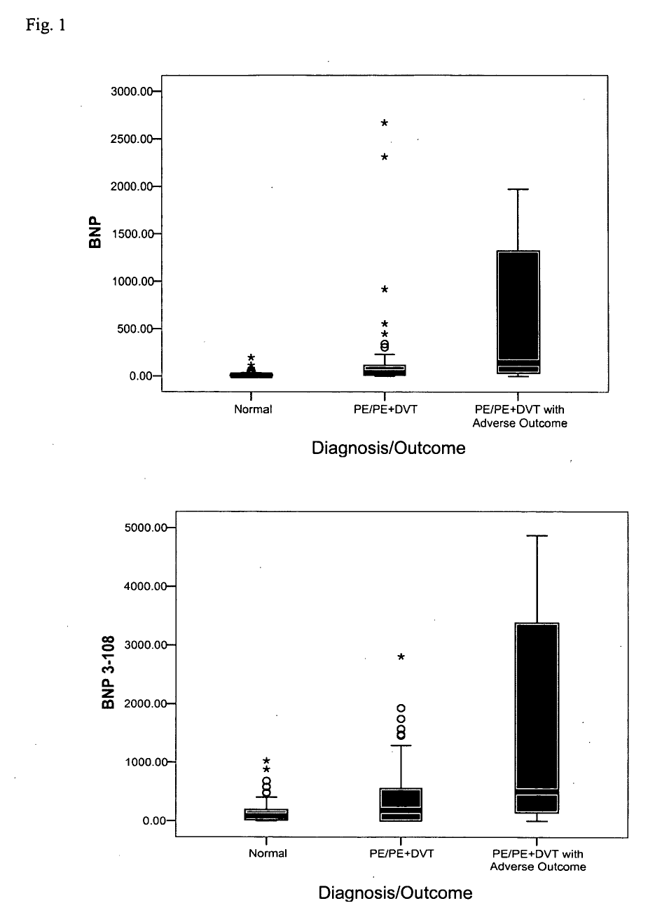

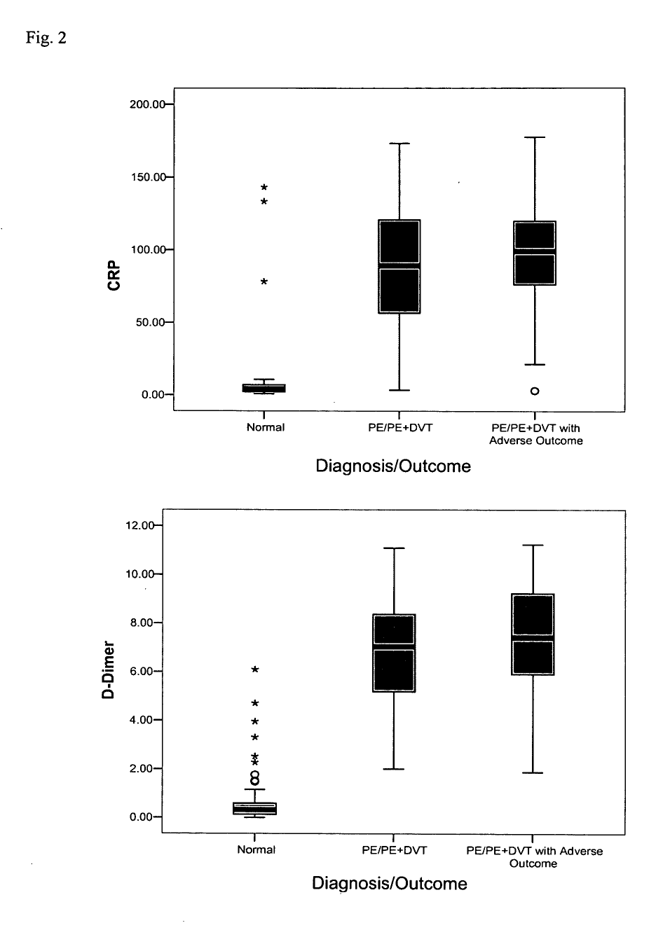

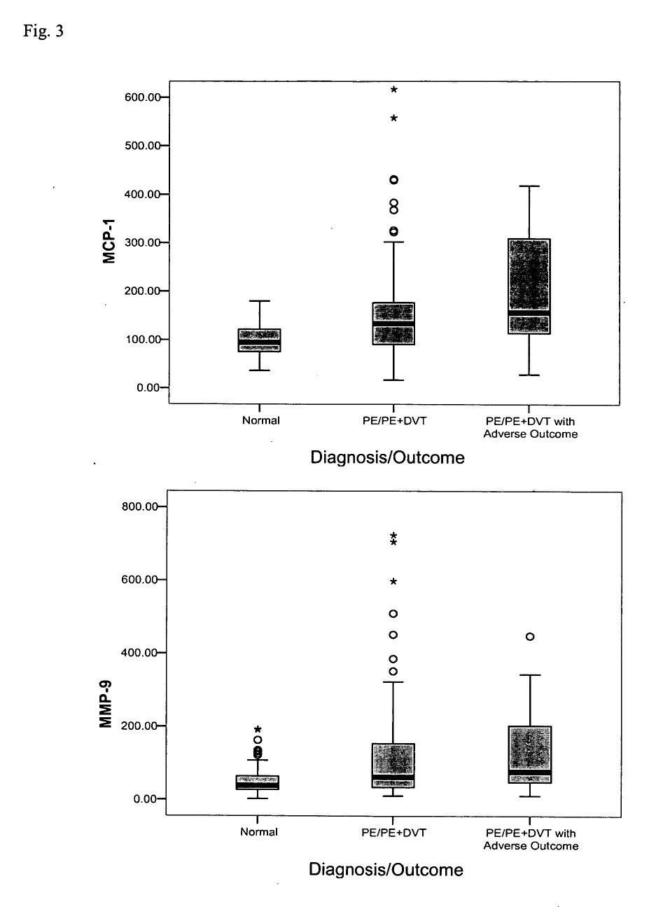

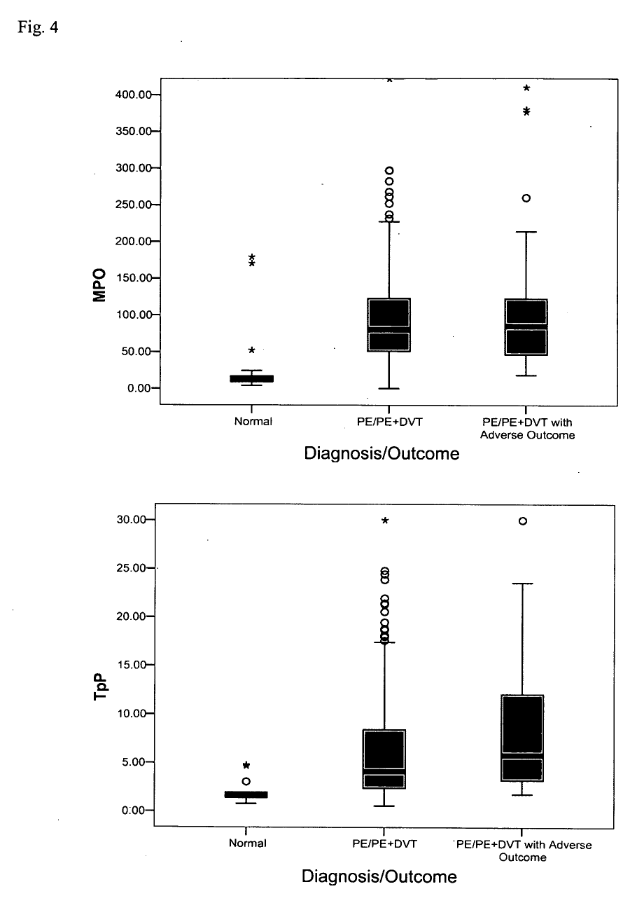

FIGS. 1-4 depict the results of certain of these measurements in “box-and-whiskers” format calculated using SPSS 10.1 software (SPSS, Inc.).

### Example 4

### Microtiter Plate-Based Biochemical Analyses

For the sandwich immunoassay in microtiter plates, a monoclonal antibody directed against a selected analyte was biotinylated using N-hydroxysuccinimide biotin (NHS-biotin) at a ratio of about 5 NHS-biotin moieties per antibody. The antibody-biotin conjugate was then added to wells of a standard avidin 384 well microtiter plate, and antibody conjugate not bound to the plate was removed. This formed the “anti-marker” in the microtiter plate. Another monoclonal antibody directed against the same analyte was conjugated to alkaline phosphatase, for example using succinimidyl 4-[N-maleimidomethyl]-cyclohexane-1-carboxylate (SMCC) and N-succinimidyl 3-[2-pyridyldithio]propionate (SPDP) (Pierce, Rockford, Ill.).

Biotinylated antibodies were pipetted into microtiter plate wells previously coated with avidin and incubated for 60 min. The solution containing unbound antibody was removed, and the wells washed with a wash buffer, consisting of 20 mM borate (pH 7.42) containing 150 mM NaCl, 0.1% sodium azide, and 0.02% Tween-20. The plasma samples (10 μL, or 20 μL for CCL4) containing added HAMA inhibitors were pipeted into the microtiter plate wells, and incubated for 60 min. The sample was then removed and the wells washed with a wash buffer. The antibody-alkaline phosphatase conjugate was then added to the wells and incubated for an additional 60 min, after which time, the antibody conjugate was removed and the wells washed with a wash buffer. A substrate, (AttoPhos®, Promega, Madison, Wis.) was added to the wells, and the rate of formation of the fluorescent product is related to the concentration of the analyte in the sample tested.

For competitive immunoassays in microtiter plates, a murine monoclonal antibody directed against a selected analyte was added to the wells of a microtiter plate and immobilized by binding to goat anti-mouse antibody that is pre-absorbed to the surface of the microtiter plate wells (Pierce, Rockford, Ill.). Any unbound murine monoclonal antibody was removed after a 60 minute incubation. This forms the “anti-marker” in the microtiter plate. A purified polypeptide that is either the same as or related to the selected analyte, and that can be bound by the monoclonal antibody, was biotinylated as described above for the biotinylation of antibodies. This biotinylated polypeptide was mixed with the sample in the presence of HAMA inhibitors, forming a mixture containing both exogenously added biotinylated polypeptide and any unlabeled analyte molecules endogenous to the sample. The amount of the monoclonal antibody and biotinylated marker added depends on various factors and was titrated empirically to obtain a satisfactory dose-response curve for the selected analyte.

This mixture was added to the microtiter plate and allowed to react with the murine monoclonal antibody for 120 minutes. After the 120 minute incubation, the unbound material was removed, and Neutralite-Alkaline Phosphatase (Southern Biotechnology; Birmingham, Ala.) was added to bind to any immobilized biotinylated polypeptide. Substrate (as described above) was added to the wells, and the rate of formation of the fluorescent product was related to the amount of biotinylated polypeptide bound, and therefore was inversely related to the endogenous amount of the analyte in the specimen.

### Example 5

### Microfluidic Device-Based Biochemical Analyses

Immunoassays were performed using microfluidic devices essentially as described in Chapter 41, entitled “Near Patient Tests: Triage® Cardiac System,” in The Immunoassay Handbook, 2^{nd} ed., David Wild, ed., Nature Publishing Group, 2001.

For sandwich immunoassays, a plasma sample is added to the microfluidic device that contains all the necessary assay reagents, including HAMA inhibitors, in dried form. The plasma passes through a filter to remove particulate matter. Plasma enters a “reaction chamber” by capillary action. This reaction chamber contains fluorescent latex particle-antibody conjugates (hereafter called FETL-antibody conjugates) appropriate to an analyte of interest, and may contain FETL-antibody conjugates to several selected analytes. The FETL-antibody conjugates dissolve into the plasma to form a reaction mixture, which is held in the reaction chamber for an incubation period (about a minute) to allow the analyte(s) of interest in the plasma to bind to the antibodies. After the incubation period, the reaction mixture moves down the detection lane by capillary action. Antibodies to the analyte(s) of interest are immobilized in discrete capture zones on the surface of a “detection lane.” Analyte/antibody-FETL complexes formed in the reaction chamber are captured on an appropriate detection zone to form a sandwich complex, while unbound FETL-antibody conjugates are washed from the detection lane into a waste chamber by excess plasma. The amount of analyte/antibody-FETL complex bound on a capture zone is quantified with a fluorometer (Triage® MeterPlus, Biosite Incorporated) and is related to the amount of the selected analyte in the plasma specimen.

For competitive immunoassays, the procedure and process is similar to that described for sandwich immunoassays, with the following exceptions. In one configuration, fluorescent latex particle-marker (FETL-marker) conjugates are provided in the reaction chamber, and are dissolved in the plasma to form a reaction mixture. This reaction mixture contains both the unlabeled analyte endogenous to the sample, and the FETL-marker conjugates. When the reaction mixture contacts the capture zone for a analyte of interest, the unlabeled endogenous analyte and the FETL-marker conjugates compete for the limited number of antibody binding sites. Thus, the amount of FETL-marker conjugate bound to the capture zone is inversely related to the amount of analyte endogenously present in the plasma specimen. In another configuration, antibody-FETL conjugates are provided in the reaction chamber as described above for sandwich assays. In this configuration, the capture zone contains immobilized marker on the surface of the detection lane. Free antibody-FETL conjugates bind to this immobilized marker on the capture zone, while antibody-FETL conjugates bound to an analyte of interest do not bind as readily or at all to this immobilized marker. Again, the amount of FETL captured in the zone is inversely related to the amount of the selected analyte in the plasma specimen. One skilled in the art will recognize that either configuration may be used depending on the characteristics and concentrations of the selected analyte(s).

The assays were calibrated using purified proteins (that is either the same as or related to the selected analyte, and that can be detected in the assay) diluted gravimetrically into EDTA plasma treated in the same manner as the sample population specimens. Endogenous levels of the analyte present in the plasma prior to addition of the purified marker protein was measured and taken into account in assigning the marker values in the calibrators. When necessary to reduce endogenous levels in the calibrators, the endogenous analyte was stripped from the plasma using standard immunoaffinity methods. Calibrators were assayed in the same manner as the sample population specimens, and the resulting data used to construct a “dose-response” curve (assay signal as a function of analyte concentration), which may be used to determine analyte concentrations from assay signals obtained from subject specimens.

### Example 6

### Use of Markers as Diagnostic Indicators in VTED

Significantly higher levels of the following biomarkers were observed in the PE and/or DVT disease group, compared to normal health controls (Non-parametric Kruskal-Wallis test for median, all p<0.001, except for MCP-1, where p<0.007). The results are presented in the following table:

TABLE 1
Normal control v. PE and/or DVT

| Subject group | | BNP | BNP₃₋₁₀₈ | Caspase-3 | CRP |
|---|---|---|---|---|---|
| Normal | N | 120 | 92 | 14 | 21 |
| | Mean | 46.2 | 145 | .704 | 20.2 |
| | Std. Deviation | 337 | 189 | .578 | 42.5 |
| | Median | 7.14 | 81.2 | .63 | 4.14 |
| | Minimum | .00 | .00 | .00 | .79 |
| | Maximum | 3694 | 1025 | 1.67 | 143 |
| PE/PE + | N | 90 | 95 | 96 | 98 |
| DVT | Mean | 523 | 2025 | 3.14 | 85.8 |
| | Std. Deviation | 1331 | 5626 | 3.92 | 40.5 |
| | Median | 33 | 180 | 1.85 | 89 |
| | Minimum | .00 | .00 | .00 | 3.43 |
| | Maximum | 5000 | 25000 | 26.8 | 173 |
| PE/PE + | N | 36 | 36 | 33 | 36 |
| DVT, | Mean | 1186 | 4505 | 3.68 | 97.0 |
| adverse | Std. Deviation | 1948 | 8166 | 3.92 | 37.4 |
| outcome | Median | 141.5 | 503 | 2.47 | 99.2 |
| | Minimum | .00 | .00 | .36 | 3.16 |
| | Maximum | 5000 | 25000 | 19.6 | 178 |
| Total | N | 246 | 223 | 143 | 155 |
| | Mean | 388 | 1650 | 3.02 | 79.5 |
| | Std. Deviation | 1183 | 5124 | 3.8 | 46.5 |
| | Median | 14.5 | 136 | 1.73 | 85.8 |
| | Minimum | .00 | .00 | .00 | .79 |
| | Maximum | 5000 | 25000 | 26.8 | 178 |
| Subject group | | D-Dimer | MCP-1 | MMP-9 | MPO |
| Normal | N | 120 | 20 | 113 | 21 |
| | Mean | .561 | 134 | 51.7 | 29.6 |
| | Std. Deviation | .888 | 158 | 36.2 | 49.1 |
| | Median | .32 | 94.8 | 38.8 | 12.3 |
| | Minimum | .00 | 36.1 | 2.7 | 4.09 |
| | Maximum | 6.09 | 782 | 193 | 179 |
| PE/PE + | N | 100 | 99 | 100 | 101 |
| DVT | Mean | 6.68 | 162 | 141 | 113 |
| | Std. Deviation | 2.30 | 149 | 268 | 147 |
| | Median | 7.04 | 133 | 62.7 | 80.5 |
| | Minimum | 2.02 | 16.7 | 11.3 | .37 |
| | Maximum | 11.1 | 1200 | 2410 | 1385 |
| PE/PE + | N | 37 | 37 | 37 | 37 |
| DVT, | Mean | 7.33 | 245 | 178 | 111 |
| adverse | Std. Deviation | 2.37 | 234 | 283 | 100 |
| outcome | Median | 7.42 | 157 | 77.5 | 85.3 |
| | Minimum | 1.89 | 28.5 | 11.34 | 18.64 |
| | Maximum | 11.2 | 1151 | 1483 | 410 |
| Total | N | 257 | 156 | 250 | 159 |
| | Mean | 3.91 | 178 | 106 | 101 |
| | Std. Deviation | 3.63 | 177 | 208 | 131 |
| | Median | 3.24 | 129 | 48.8 | 78 |
| | Minimum | .00 | 16.7 | 2.7 | .37 |
| | Maximum | 11.2 | 1200 | 2410 | 1385 |

The markers were also subjected to ROC analysis, with the following results:

TABLE 2
Normal control v. PE and/or DVT

| Marker | n(normal) | n(disease) | ROC area |
|---|---|---|---|
| BNP | 120 | 126 | 0.77 |
| BNP₃₋₁₀₈ | 92 | 131 | 0.67 |
| Caspase-3 | 14 | 129 | 0.85 |
| CRP | 21 | 134 | 0.89 |
| D-dimer | 120 | 137 | 0.99 |
| MCP-1 | 20 | 136 | 0.66 |
| MMP-9 | 113 | 137 | 0.69 |
| MPO | 21 | 138 | 0.89 |
| TpP | 13 | 136 | 0.81 |

### Example 7

### Use of Markers as Prognostic Indicator in VETD

There were also significantly higher levels of BNP, BNP₃₋₁₀₈, and MCP-1 found in PE and/or DVT subjects who experienced an adverse outcome, compared to PE and/or DVT subjects without adverse outcome. Wilcoxon test p-values for the comparisons are 0.002, 0.004, and 0.0312, respectively.

Table 3: PE and/or DVT; normal=no adverse outcome; disease=adverse outcome as defined above in Example 2.

The markers were also subjected to ROC analysis, with the following results:

TABLE 4
PE and/or DVT; normal = no adverse outcome; disease = adverse
outcome as defined above in Example 3

| Marker | n(normal) | n(disease) | ROC area |
|---|---|---|---|
| BNP | 90 | 36 | 0.67 |
| BNP₃₋₁₀₈ | 95 | 36 | 0.66 |
| Caspase-3 | 96 | 33 | 0.55 |
| CRP | 98 | 36 | 0.57 |
| D-dimer | 100 | 37 | 0.58 |
| MCP-1 | 99 | 37 | 0.62 |
| MMP-9 | 100 | 37 | 0.57 |
| MPO | 101 | 37 | 0.49 |
| TpP | 99 | 37 | 0.59 |

The prevalence of adverse outcome in the study group was 28%. If a patient had a history of prior cardiopulmonary disease, a positive medical panel (defined as either persistent hypoxemia defined as a pulse oximetry reading <95% while the patient breathed room air, or evidence of pulmonary hypertension from 12-lead electrocardiography defined as a Daniel score >8, or evidence of cardiac cell necrosis from the serum troponin T measurement concentration defined as >0.1 ng/mL), a D-dimer >8 ug/mL, B-type natriuretic peptide >90 pg/mL, the probability of an adverse outcome (i.e., the predictive value positive) increased to 57%, 42%, 50%, 57%, respectively. A three marker panel consisting of a combination of troponin T>0.1 ng/mL, D-dimer >8 μg/mL, or B-type natriuretic peptide >90 pg/mL yielded sensitivity, specificity and accuracy of 72% (95% CI: 57 to 84%), 68% (58 to 76%) and 69% (61 to 76%) respectively.

### Example 8

### Panels for Risk Stratification in VETD

Using the methods described in PCT application no. US03/41426, filed Dec. 23, 2003, exemplary panels for risk stratification is VETD were identified. Starting with a large number of potential markers, an iterative procedure was applied. In this procedure, individual threshold concentrations for the markers are not used as cutoffs per se, but are used as values to which the assay values for each patient are compared and normalized. A window factor was used to calculate the minimum and maximum values above and below the cutoff. Assay values above the maximum are set to the maximum and assay values below the minimum are set to the minimum. The absolute values of the weights for the individual markers adds up to 1. A negative weight for a marker implies that the assay values for the control group are higher than those for the diseased group.

A “panel response” is calculated using the cutoff, window, and weighting factors. The panel responses for the entire population of patients and controls are subjected to ROC analysis as is commonly performed for individual markers, and a “panel response” cutoff is selected to yield the desired sensitivity and specificity for the panel. After each set of iterations, the weakest contributors to the equation may be eliminated and the iterative process started again with the reduced number of markers. This process is continued until a minimum number of markers that will still result in acceptable sensitivity and specificity of the panel is obtained.

In the present examples, the “diseased” dataset represents a population of subjects diagnosed as having PE and/or DVT and an adverse outcome as defined above in Example 3, while the “normal” dataset represents a population of subjects diagnosed as having PE and/or DVT and no adverse outcome. Samples were obtained for these subjects at hospital admission.

TABLE 5
PE and/or DVT; normal = no adverse outcome; disease = adverse
outcome as defined above in Example 2.

| | Panel # | | | |
|---|---|---|---|---|
| | 1 | 2 | 3 | 4 |
| Markers in panel | TpP, BNP, | TpP, BNP, | TpP, BNP, | TpP, MCP-1, |
| | MCP-1, CRP, | MCP-1, CRP, | MCP-1, CRP, | CRP, D-dimer, |
| | D-dimer, | caspase-3 | D-dimer, | caspase-3, |
| | caspase-3, | | caspase-3, | BNP₃₋₁₀₈, MMP- |
| | BNP₃₋₁₀₈, MMP- | | MMP-9, MPO | 9, MPO |
| | 9, MPO | | | |
| “Normal” n | 83 | 85 | 85 | 88 |
| “Disease” n | 31 | 32 | 32 | 31 |
| Ave ROC Area | 0.822 | 0.816 | 0.824 | 0.807 |
| SD(%) | 0.021 | 0.019 | 0.019 | 0.022 |
| Ave Sens @ 92.5% | 54% | 55% | 57% | 50% |
| Spec | | | | |
| SD(%) | 7.8 | 8.9 | 5.8 | 7.0 |
| Ave Spec @ 92.5% | 63% | 59% | 61% | 61% |
| Sens | | | | |
| SD(%) | 8.1 | 6.8 | 8.6 | 7.3 |
| | Panel # | | | |
| | 5 | 6 | 7 | 8 |
| Markers in panel | BNP, MCP-1, | BNP, MCP-1, | BNP, MCP-1, | BNP, MCP-1, |
| | CRP, D-dimer, | CRP, D-dimer, | CRP, D-dimer, | CRP, D-dimer, |
| | caspase-3, | BNP₃₋₁₀₈ | caspase-3, | caspase-3 |
| | BNP₃₋₁₀₈, MMP- | | MMP-9, MPO | |
| | 9, MPO | | | |
| “Normal” n | 83 | 85 | 85 | 85 |
| “Disease” n | 31 | 34 | 32 | 32 |
| Ave ROC Area | 0.807 | 0.781 | 0.799 | 0.805 |
| SD(%) | 0.019 | 0.021 | 0.021 | 0.022 |
| Ave Sens @ 92.5% | 53% | 41% | 54% | 54% |
| Spec | | | | |
| SD(%) | 6.3 | 5.8 | 7.6 | 7.3 |
| Ave Spec @ 92.5% | 56% | 49% | 50% | 49% |
| Sens | | | | |
| SD(%) | 7.3 | 7.3 | 5.2 | 4.5 |

### Example 9

### Panels for Diagnosis of VETD

TABLE 6
Normal control v. PE and/or DVT

| | Panel # | | | |
|---|---|---|---|---|
| | 9 | 10 | 11 | 12 |
| Markers in panel | TpP, BNP, | TpP, MPO, D- | TpP, BNP, | TpP, CRP, D- |
| | MCP-1, CRP, | dimer, CRP, | MCP-1, CRP, | dimer, caspase- |
| | D-dimer, | caspase-3, | D-dimer, | 3, BNP₃₋₁₀₈, |
| | caspase-3, | | caspase-3, | MMP-9, MPO |
| | BNP₃₋₁₀₈, MMP- | | MMP-9, MPO | |
| | 9, MPO | | | |
| “Normal” n | 13 | 13 | 13 | 13 |
| “Disease” n | 114 | 126 | 117 | 119 |
| Ave ROC Area | 0.999 | 0.997 | 0.997 | 0.999 |
| SD(%) | 0.001 | 0.004 | 0.004 | 0.001 |
| Ave Sens @ 92.5% | 99% | 98% | 98% | 99% |
| Spec | | | | |
| SD(%) | 0.7 | 1.9 | 1.8 | 0.7 |
| Ave Spec @ 92.5% | 100% | 100% | 100% | 100% |
| Sens | | | | |
| SD(%) | 0.0 | 0.0 | 0.0 | 0.0 |

| | Panel # | | |
|---|---|---|---|
| | 13 | 14 | 15 |
| Markers in panel | MCP-1, BNP, | CRP, D-dimer, | BNP, MCP-1, |
| | CRP, D-dimer, | caspase-3, | CRP, D-dimer, |
| | caspase-3, | MMP-9, MPO | caspase-3, |
| | BNP₃₋₁₀₈, MMP- | | MMP-9, MPO |
| | 9, MPO | | |
| “Normal” n | 13 | 13 | 13 |
| “Disease” n | 114 | 126 | 117 |
| Ave ROC Area | 0.997 | 0.996 | 0.998 |
| SD(%) | 0.002 | 0.002 | 0.001 |
| Ave Sens @ 92.5% | 99% | 98% | 99% |
| Spec | | | |
| SD(%) | 0.6 | 0.6 | 0.5 |
| Ave Spec @ 92.5% | 100% | 100% | 100% |
| Sens | | | |
| SD(%) | 0.0 | 0.0 | 0.0 |

### Example 10

### Subject Population

For Examples 11 and 12 below, samples were collected from 439 human subjects enrolled in one of two studies on presentation at an emergency medical facility. Inclusion criteria required that the subjects were suspected of having pulmonary embolism or deep vein thrombosis. Exclusion criteria included those already treated for the current episode of PE or DVT, those with active heart failure, a history of renal disease requiring dialysis, or experiencing an MI, PTCA, CABG or Unstable Angina within the past month. A population of apparently healthy indivduals not enrolled in the study (n=49) were selected to serve as normal control samples.

The diagnoses of the 439 enrolled subjects, provided by standard clinical methods in use at the medical facility, were as follows:

57 PE+ 382 PE−

51 DVT+ 381 DVT− 7 DVT status uncertain

For assessment of prognosis, subjects were followed for 45 days. The following outcomes were observed:

Among the 57 PE+ patients: 4 deaths in the follow up period; 23 combined adverse outcomes defined as ICU admissions on presentation, recurring PE in the follow up period and/or deaths in the follow up period.

Among the 439 enrolled subjects: 11 deaths in the follow up period; 34 combined adverse outcomes by the same definition.

### Example 11

### Use of Individual Markers as Diagnostic and Prognostic Indicators in VTED

The subject population was subdivided into the following groups (1) through (4) for diagnostic comparisons: (1) normal subjects gathered outside of study enrollment criteria; (2) PE+ (positive for pulmonary embolism, and positive, negative, or undetermined for DVT); (3) DVT+ (positive for DVT, and positive or negative for pulmonary embolism), and (4) PE− (negative for pulmonary embolism, and positive, negative, or undetermined for DVT). The subject population was further subdivided into the following groups (5) through (12) for prognostic comparisons: (5) PE+ and alive through the followup period; (6) PE+, and death within the follow up period; (7) all enrolled subjects, and alive at 45 days; (8) all enrolled subjects, and death within the follow up period; (9) PE+, and no combined adverse outcome through the follow up period; (10) PE+, and one or more of the combined adverse outcomes within the follow up period; (11) all enrolled subjects, and no combined adverse outcome through the follow up period; and (12) all enrolled subjects, and one or more of the combined adverse outcomes within the follow up period. Marker assays were performed as described above in Examples 3, 4, and 5. The results of the assays in each of these groups are presented in the following Table 7:

| Subject | | | | | Basic |
|---|---|---|---|---|---|
| population | | Acidic Calponin | Adrenomedullin | Angiopoietin-4 | Calponin |
| (1) | N | 46 | 49 | 49 | 49 |
| | Mean | 1.11 | 42.18 | 4.40 | 18.62 |
| | Std Dev | 0.56 | 29.79 | 3.41 | 14.73 |
| | Median | 0.96 | 34.15 | 3.61 | 14.53 |
| | Min | 0.52 | 16.18 | 0.90 | 5.00 |
| | Max | 3.20 | 164.72 | 17.93 | 98.26 |
| (2) | N | 55 | 57 | 57 | 57 |
| | Mean | 1.79 | 84.78 | 3.94 | 69.57 |
| | Std Dev | 1.15 | 78.21 | 2.09 | 59.42 |
| | Median | 1.58 | 59.04 | 4.00 | 56.77 |
| | Min | 0.03 | 0.50 | 0.90 | 5.00 |
| | Max | 5.15 | 424.74 | 10.94 | 337.45 |
| (3) | N | 50 | 51 | 51 | 51 |
| | Mean | 2.03 | 103.60 | 4.29 | 81.28 |
| | Std Dev | 1.88 | 113.63 | 2.98 | 76.55 |
| | Median | 1.57 | 68.44 | 3.87 | 60.54 |
| | Min | 0.03 | 14.81 | 0.90 | 13.67 |
| | Max | 12.29 | 734.85 | 16.87 | 399.23 |
| (4) | N | 382 | 382 | 380 | 382 |
| | Mean | 1.75 | 70.70 | 7.47 | 64.31 |
| | Std Dev | 1.37 | 80.21 | 26.71 | 58.16 |
| | Median | 1.45 | 49.42 | 3.80 | 47.01 |
| | Min | 0.03 | 0.50 | 0.90 | 5.00 |
| | Max | 15.92 | 964.35 | 449.16 | 399.23 |
| (5) | N | 51 | 53 | 53 | 53 |
| | Mean | 1.69 | 77.01 | 3.99 | 63.05 |
| | Std Dev | 1.02 | 62.60 | 2.13 | 45.56 |
| | Median | 1.55 | 58.75 | 4.04 | 54.23 |
| | Min | 0.03 | 0.50 | 0.90 | 5.00 |
| | Max | 4.38 | 340.03 | 10.94 | 209.90 |
| (6) | N | 4 | 4 | 4 | 4 |
| | Mean | 3.17 | 187.78 | 3.26 | 155.94 |
| | Std Dev | 1.88 | 176.19 | 1.37 | 138.73 |
| | Median | 3.14 | 144.72 | 3.00 | 127.29 |
| | Min | 1.25 | 36.95 | 1.89 | 31.72 |
| | Max | 5.15 | 424.74 | 5.14 | 337.45 |
| (7) | N | 426 | 428 | 426 | 428 |
| | Mean | 1.74 | 71.48 | 7.02 | 64.05 |
| | Std Dev | 1.34 | 78.81 | 25.25 | 57.02 |
| | Median | 1.45 | 49.90 | 3.83 | 47.03 |
| | Min | 0.03 | 0.50 | 0.90 | 5.00 |
| | Max | 15.92 | 964.35 | 449.16 | 399.23 |
| (8) | N | 11 | 11 | 11 | 11 |
| | Mean | 2.31 | 113.40 | 6.47 | 101.54 |
| | Std Dev | 1.43 | 114.98 | 5.32 | 91.97 |
| | Median | 2.00 | 72.22 | 4.72 | 65.15 |
| | Min | 0.03 | 36.95 | 1.89 | 24.80 |
| | Max | 5.15 | 424.74 | 16.87 | 337.45 |
| (9) | N | 33 | 34 | 34 | 34 |
| | Mean | 1.70 | 79.10 | 4.02 | 60.54 |
| | Std Dev | 0.94 | 71.98 | 2.25 | 46.64 |
| | Median | 1.61 | 59.06 | 4.11 | 46.44 |
| | Min | 0.03 | 0.50 | 0.90 | 5.00 |
| | Max | 4.38 | 340.03 | 10.94 | 209.90 |
| (10) | N | 22 | 23 | 23 | 23 |
| | Mean | 1.94 | 93.19 | 3.82 | 82.93 |
| | Std Dev | 1.42 | 87.60 | 1.87 | 73.58 |
| | Median | 1.29 | 59.04 | 3.87 | 64.76 |
| | Min | 0.03 | 23.60 | 1.09 | 14.66 |
| | Max | 5.15 | 424.74 | 9.27 | 337.45 |
| (11) | N | 404 | 405 | 403 | 405 |
| | Mean | 1.74 | 70.84 | 7.21 | 63.44 |
| | Std Dev | 1.35 | 80.06 | 25.95 | 57.64 |
| | Median | 1.45 | 49.56 | 3.83 | 45.81 |
| | Min | 0.03 | 0.50 | 0.90 | 5.00 |
| | Max | 15.92 | 964.35 | 449.16 | 399.23 |
| (12) | N | 33 | 34 | 34 | 34 |
| | Mean | 1.98 | 92.68 | 4.67 | 83.41 |
| | Std Dev | 1.29 | 77.70 | 3.59 | 63.51 |
| | Median | 1.73 | 66.14 | 3.96 | 65.07 |
| | Min | 0.03 | 23.60 | 0.90 | 14.66 |
| | Max | 5.15 | 424.74 | 16.87 | 337.45 |

| Subject | | | | | |
|---|---|---|---|---|---|
| population | | BNP | BMP4 | BNP₁₋₁₀₈ | BNP₃₋₁₀₈ |
| (1) | N | 47 | 49 | 47 | 49 |
| | Mean | 30.21 | 0.65 | 91.44 | 76.16 |
| | Std Dev | 32.21 | 0.21 | 167.46 | 90.07 |
| | Median | 19.27 | 0.59 | 40.00 | 33.43 |
| | Min | 2.20 | 0.33 | 40.00 | 25.00 |
| | Max | 156.95 | 1.47 | 993.22 | 479.99 |
| (2) | N | 51 | 57 | 57 | 57 |
| | Mean | 130.54 | 0.73 | 208.35 | 299.65 |
| | Std Dev | 243.04 | 0.37 | 236.45 | 521.73 |
| | Median | 50.32 | 0.67 | 124.82 | 115.56 |
| | Min | 2.20 | 0.28 | 40.00 | 25.00 |
| | Max | 1476.93 | 2.74 | 1156.62 | 2701.54 |
| (3) | N | 39 | 51 | 51 | 51 |
| | Mean | 168.72 | 0.78 | 272.87 | 355.66 |
| | Std Dev | 287.16 | 0.34 | 420.43 | 566.87 |
| | Median | 57.14 | 0.68 | 130.31 | 144.30 |
| | Min | 2.20 | 0.41 | 40.00 | 25.00 |
| | Max | 1476.93 | 2.35 | 2373.94 | 2701.54 |
| (4) | N | 271 | 382 | 382 | 382 |
| | Mean | 87.74 | 0.71 | 190.35 | 209.29 |
| | Std Dev | 218.06 | 0.39 | 366.42 | 469.42 |
| | Median | 17.56 | 0.65 | 54.97 | 50.29 |
| | Min | 2.20 | 0.05 | 40.00 | 25.00 |
| | Max | 2357.71 | 5.25 | 3113.84 | 5912.60 |
| (5) | N | 48 | 53 | 53 | 53 |
| | Mean | 129.00 | 0.69 | 199.00 | 292.77 |
| | Std Dev | 248.03 | 0.24 | 231.13 | 526.05 |
| | Median | 47.90 | 0.67 | 124.82 | 115.56 |
| | Min | 2.20 | 0.28 | 40.00 | 25.00 |
| | Max | 1476.93 | 1.66 | 1156.62 | 2701.54 |
| (6) | N | 3 | 4 | 4 | 4 |
| | Mean | 155.29 | 1.28 | 332.21 | 390.83 |
| | Std Dev | 173.40 | 1.01 | 309.27 | 521.98 |
| | Median | 66.79 | 0.95 | 341.61 | 194.35 |
| | Min | 44.00 | 0.48 | 40.00 | 25.00 |
| | Max | 355.09 | 2.74 | 605.64 | 1149.63 |
| (7) | N | 312 | 428 | 428 | 428 |
| | Mean | 90.85 | 0.71 | 188.28 | 214.38 |
| | Std Dev | 217.00 | 0.38 | 349.15 | 468.88 |
| | Median | 17.83 | 0.65 | 57.66 | 54.22 |
| | Min | 2.20 | 0.05 | 40.00 | 25.00 |
| | Max | 2357.71 | 5.25 | 3113.84 | 5912.60 |
| (8) | N | 10 | 11 | 11 | 11 |
| | Mean | 208.94 | 0.98 | 364.34 | 479.52 |
| | Std Dev | 348.24 | 0.62 | 437.59 | 705.31 |
| | Median | 78.15 | 0.82 | 103.86 | 253.30 |
| | Min | 5.85 | 0.48 | 40.00 | 25.00 |
| | Max | 1149.52 | 2.74 | 1400.87 | 2375.00 |
| (9) | N | 30 | 34 | 34 | 34 |
| | Mean | 125.74 | 0.69 | 173.65 | 251.53 |
| | Std Dev | 283.58 | 0.25 | 204.86 | 491.46 |
| | Median | 21.74 | 0.66 | 84.47 | 84.14 |
| | Min | 2.20 | 0.28 | 40.00 | 25.00 |
| | Max | 1476.93 | 1.66 | 836.69 | 2701.54 |
| (10) | N | 21 | 23 | 23 | 23 |
| | Mean | 137.40 | 0.80 | 259.65 | 370.77 |
| | Std Dev | 176.01 | 0.49 | 273.40 | 567.19 |
| | Median | 66.79 | 0.70 | 169.67 | 159.17 |
| | Min | 2.20 | 0.37 | 40.00 | 25.00 |
| | Max | 722.74 | 2.74 | 1156.62 | 2556.84 |
| (11) | N | 290 | 405 | 405 | 405 |
| | Mean | 84.60 | 0.71 | 183.67 | 201.90 |
| | Std Dev | 216.95 | 0.38 | 352.71 | 457.69 |
| | Median | 17.32 | 0.65 | 52.89 | 49.29 |
| | Min | 2.20 | 0.05 | 40.00 | 25.00 |
| | Max | 2357.71 | 5.25 | 3113.84 | 5912.60 |
| (12) | N | 32 | 34 | 34 | 34 |
| | Mean | 184.37 | 0.82 | 300.13 | 448.82 |
| | Std Dev | 252.79 | 0.43 | 331.03 | 629.11 |
| | Median | 83.68 | 0.75 | 156.62 | 193.76 |
| | Min | 2.20 | 0.37 | 40.00 | 25.00 |
| | Max | 1149.52 | 2.74 | 1400.87 | 2556.84 |

| Subject | | | | | |
|---|---|---|---|---|---|
| population | | BNP₇₉₋₁₀₈ | CCL11 | CGRP | CK-BB |
| (1) | N | 47 | 49 | 49 | 49 |
| | Mean | 7.26 | 46.23 | 489.74 | 0.27 |
| | Std Dev | 11.50 | 40.76 | 442.74 | 0.26 |
| | Median | 4.00 | 33.30 | 351.60 | 0.15 |
| | Min | 4.00 | 8.00 | 119.10 | 0.15 |
| | Max | 63.96 | 189.26 | 2425.11 | 1.23 |
| (2) | N | 57 | 57 | 57 | 57 |
| | Mean | 13.07 | 73.52 | 634.54 | 0.66 |
| | Std Dev | 17.78 | 76.54 | 591.47 | 0.73 |
| | Median | 4.00 | 58.92 | 429.44 | 0.37 |
| | Min | 4.00 | 8.00 | 95.00 | 0.15 |
| | Max | 86.31 | 507.99 | 3213.50 | 4.41 |
| (3) | N | 51 | 51 | 51 | 51 |
| | Mean | 28.04 | 92.80 | 675.40 | 0.83 |
| | Std Dev | 84.72 | 152.41 | 714.95 | 1.21 |
| | Median | 4.00 | 49.61 | 409.71 | 0.52 |
| | Min | 4.00 | 8.00 | 95.00 | 0.15 |
| | Max | 576.84 | 922.17 | 4133.24 | 7.43 |
| (4) | N | 382 | 382 | 382 | 382 |
| | Mean | 14.92 | 69.50 | 578.01 | 0.70 |
| | Std Dev | 46.64 | 109.73 | 671.04 | 0.97 |
| | Median | 4.00 | 42.13 | 379.02 | 0.35 |
| | Min | 4.00 | 8.00 | 95.00 | 0.15 |
| | Max | 576.84 | 992.95 | 5719.74 | 7.86 |
| (5) | N | 53 | 53 | 53 | 53 |
| | Mean | 12.52 | 72.99 | 591.04 | 0.63 |
| | Std Dev | 17.73 | 77.48 | 483.86 | 0.72 |
| | Median | 4.00 | 58.92 | 429.44 | 0.37 |
| | Min | 4.00 | 8.00 | 95.00 | 0.15 |
| | Max | 86.31 | 507.99 | 1848.19 | 4.41 |
| (6) | N | 4 | 4 | 4 | 4 |
| | Mean | 20.36 | 80.54 | 1210.92 | 1.07 |
| | Std Dev | 19.40 | 72.28 | 1412.73 | 0.79 |
| | Median | 17.63 | 49.79 | 767.59 | 1.05 |
| | Min | 4.00 | 35.26 | 95.00 | 0.35 |
| | Max | 42.16 | 187.32 | 3213.50 | 1.81 |
| (7) | N | 428 | 428 | 428 | 428 |
| | Mean | 14.23 | 69.64 | 578.68 | 0.68 |
| | Std Dev | 43.82 | 106.36 | 652.24 | 0.93 |
| | Median | 4.00 | 42.77 | 379.02 | 0.35 |
| | Min | 4.00 | 8.00 | 95.00 | 0.15 |
| | Max | 576.84 | 992.95 | 5719.74 | 7.86 |
| (8) | N | 11 | 11 | 11 | 11 |
| | Mean | 32.33 | 85.10 | 845.14 | 1.35 |
| | Std Dev | 48.24 | 91.69 | 943.28 | 1.17 |
| | Median | 4.00 | 46.05 | 411.70 | 1.00 |
| | Min | 4.00 | 8.60 | 95.00 | 0.15 |
| | Max | 136.04 | 320.77 | 3213.50 | 4.15 |
| (9) | N | 34 | 34 | 34 | 34 |
| | Mean | 12.20 | 54.12 | 495.71 | 0.63 |
| | Std Dev | 20.17 | 38.75 | 363.31 | 0.84 |
| | Median | 4.00 | 38.12 | 411.40 | 0.25 |
| | Min | 4.00 | 8.00 | 95.00 | 0.15 |
| | Max | 86.31 | 193.63 | 1635.82 | 4.41 |
| (10) | N | 23 | 23 | 23 | 23 |
| | Mean | 14.37 | 102.19 | 839.77 | 0.71 |
| | Std Dev | 13.83 | 105.92 | 786.56 | 0.53 |
| | Median | 7.07 | 75.23 | 429.44 | 0.57 |
| | Min | 4.00 | 8.00 | 95.00 | 0.15 |
| | Max | 52.29 | 507.99 | 3213.50 | 1.94 |
| (11) | N | 405 | 405 | 405 | 405 |
| | Mean | 14.29 | 67.86 | 569.10 | 0.67 |
| | Std Dev | 44.96 | 106.29 | 652.67 | 0.95 |
| | Median | 4.00 | 41.45 | 381.61 | 0.33 |
| | Min | 4.00 | 8.00 | 95.00 | 0.15 |
| | Max | 576.84 | 992.95 | 5719.74 | 7.86 |
| (12) | N | 34 | 34 | 34 | 34 |
| | Mean | 19.33 | 95.77 | 778.97 | 0.92 |
| | Std Dev | 29.82 | 99.67 | 735.18 | 0.89 |
| | Median | 6.82 | 68.93 | 384.34 | 0.72 |
| | Min | 4.00 | 8.00 | 95.00 | 0.15 |
| | Max | 136.04 | 507.99 | 3213.50 | 4.15 |

| Subject | | | | | |
|---|---|---|---|---|---|
| population | | CK-MB | CRP | D-Dimer | sELAF |
| (1) | N | 33 | 49 | 47 | 49 |
| | Mean | 1.63 | 33.95 | 369.98 | 13.61 |
| | Std Dev | 1.39 | 70.19 | 848.34 | 5.71 |
| | Median | 1.00 | 11.42 | 118.34 | 12.10 |
| | Min | 1.00 | 1.84 | 6.60 | 5.18 |
| | Max | 8.33 | 400.00 | 5415.26 | 27.61 |
| (2) | N | 51 | 57 | 51 | 57 |
| | Mean | 1.90 | 99.02 | 3856.43 | 25.61 |
| | Std Dev | 2.17 | 120.23 | 1888.73 | 20.35 |
| | Median | 1.00 | 50.30 | 4133.42 | 20.86 |
| | Min | 1.00 | 1.50 | 72.66 | 1.00 |
| | Max | 12.72 | 400.00 | 5829.00 | 106.88 |
| (3) | N | 39 | 51 | 39 | 51 |
| | Mean | 1.70 | 103.34 | 3915.36 | 30.30 |
| | Std Dev | 1.48 | 128.03 | 1955.90 | 26.54 |
| | Median | 1.00 | 54.43 | 4165.86 | 23.80 |
| | Min | 1.00 | 1.50 | 348.16 | 3.47 |
| | Max | 8.15 | 400.00 | 5829.00 | 151.55 |
| (4) | N | 271 | 382 | 271 | 382 |
| | Mean | 2.46 | 35.45 | 960.50 | 21.54 |
| | Std Dev | 6.27 | 78.61 | 1337.44 | 18.67 |
| | Median | 1.30 | 12.26 | 426.15 | 15.24 |
| | Min | 1.00 | 1.50 | 6.60 | 1.00 |
| | Max | 90.80 | 400.00 | 5829.00 | 151.55 |
| (5) | N | 48 | 53 | 48 | 53 |
| | Mean | 1.95 | 88.45 | 3789.52 | 23.55 |
| | Std Dev | 2.23 | 110.37 | 1902.46 | 17.30 |
| | Median | 1.00 | 46.54 | 4079.03 | 20.77 |
| | Min | 1.00 | 1.50 | 72.66 | 1.00 |
| | Max | 12.72 | 400.00 | 5829.00 | 106.88 |
| (6) | N | 3 | 4 | 3 | 4 |
| | Mean | 1.00 | 239.07 | 4927.06 | 52.95 |
| | Std Dev | 0.00 | 174.82 | 1517.00 | 38.34 |
| | Median | 1.00 | 253.73 | 5776.54 | 57.68 |
| | Min | 1.00 | 48.84 | 3175.64 | 10.90 |
| | Max | 1.00 | 400.00 | 5829.00 | 85.55 |
| (7) | N | 312 | 428 | 312 | 428 |
| | Mean | 2.40 | 42.17 | 1385.29 | 21.67 |
| | Std Dev | 5.90 | 85.41 | 1765.65 | 18.35 |
| | Median | 1.24 | 14.13 | 508.37 | 16.16 |
| | Min | 1.00 | 1.50 | 6.60 | 1.00 |
| | Max | 90.80 | 400.00 | 5829.00 | 151.55 |
| (8) | N | 10 | 11 | 10 | 11 |
| | Mean | 1.35 | 103.40 | 2476.47 | 37.64 |
| | Std Dev | 0.59 | 144.72 | 2113.40 | 32.11 |
| | Median | 1.00 | 35.00 | 2324.54 | 29.98 |
| | Min | 1.00 | 6.84 | 295.38 | 7.06 |
| | Max | 2.56 | 400.00 | 5829.00 | 85.55 |
| (9) | N | 30 | 34 | 30 | 34 |
| | Mean | 2.10 | 75.28 | 3444.93 | 22.66 |
| | Std Dev | 2.62 | 92.44 | 1971.76 | 19.37 |
| | Median | 1.00 | 36.47 | 3272.81 | 20.18 |
| | Min | 1.00 | 1.50 | 72.66 | 1.00 |
| | Max | 12.72 | 400.00 | 5829.00 | 106.88 |
| (10) | N | 21 | 23 | 21 | 23 |
| | Mean | 1.60 | 134.13 | 4444.29 | 29.98 |
| | Std Dev | 1.30 | 147.71 | 1632.20 | 21.39 |
| | Median | 1.00 | 62.56 | 5386.38 | 22.26 |
| | Min | 1.00 | 6.72 | 1336.69 | 7.88 |
| | Max | 5.31 | 400.00 | 5829.00 | 85.55 |
| (11) | N | 290 | 405 | 290 | 405 |
| | Mean | 2.14 | 37.95 | 1186.31 | 21.32 |
| | Std Dev | 3.20 | 79.44 | 1594.50 | 18.42 |
| | Median | 1.28 | 13.24 | 449.78 | 15.89 |
| | Min | 1.00 | 1.50 | 6.60 | 1.00 |
| | Max | 38.65 | 400.00 | 5829.00 | 151.55 |
| (12) | N | 32 | 34 | 32 | 34 |
| | Mean | 4.43 | 112.24 | 3529.48 | 30.95 |
| | Std Dev | 15.81 | 139.45 | 2034.31 | 22.63 |
| | Median | 1.00 | 53.48 | 3366.38 | 22.46 |
| | Min | 1.00 | 6.72 | 22.32 | 7.06 |
| | Max | 90.80 | 400.00 | 5829.00 | 85.55 |

| Subject | | | | | |
|---|---|---|---|---|---|
| population | | Endothelin-1 | GSTP | hFABP | IL-1ra |
| (1) | N | 45 | 42 | 49 | 49 |
| | Mean | 188.74 | 3.45 | 5.09 | 514.97 |
| | Std Dev | 173.05 | 2.83 | 3.24 | 440.06 |
| | Median | 146.49 | 2.75 | 4.20 | 333.65 |
| | Min | 55.00 | 0.16 | 2.32 | 87.98 |
| | Max | 1161.58 | 16.37 | 22.40 | 1795.71 |
| (2) | N | 56 | 55 | 57 | 57 |
| | Mean | 337.11 | 7.76 | 6.94 | 1068.88 |
| | Std Dev | 341.75 | 6.78 | 7.40 | 981.02 |
| | Median | 215.59 | 6.74 | 4.24 | 713.23 |
| | Min | 55.00 | 0.16 | 1.41 | 254.65 |
| | Max | 1882.98 | 43.35 | 46.42 | 4452.08 |
| (3) | N | 51 | 49 | 51 | 51 |
| | Mean | 650.43 | 8.38 | 8.47 | 1108.40 |
| | Std Dev | 2497.03 | 7.87 | 9.09 | 934.28 |
| | Median | 205.22 | 5.42 | 5.26 | 842.85 |
| | Min | 55.00 | 0.16 | 1.60 | 240.16 |
| | Max | 18000.00 | 43.35 | 46.42 | 5045.54 |
| (4) | N | 374 | 363 | 382 | 381 |
| | Mean | 350.34 | 11.49 | 7.19 | 1343.52 |
| | Std Dev | 1002.35 | 9.41 | 12.07 | 4533.40 |
| | Median | 173.92 | 8.77 | 4.71 | 655.96 |
| | Min | 55.00 | 0.16 | 0.10 | 112.92 |
| | Max | 18000.00 | 56.60 | 152.03 | 65000.00 |
| (5) | N | 52 | 51 | 53 | 53 |
| | Mean | 323.55 | 7.66 | 6.83 | 1002.21 |
| | Std Dev | 317.92 | 6.77 | 7.52 | 901.91 |
| | Median | 215.59 | 6.07 | 3.86 | 713.23 |
| | Min | 55.00 | 0.16 | 1.41 | 254.65 |
| | Max | 1882.98 | 43.35 | 46.42 | 4452.08 |
| (6) | N | 4 | 4 | 4 | 4 |
| | Mean | 513.48 | 8.98 | 8.42 | 1952.26 |
| | Std Dev | 615.08 | 7.77 | 6.18 | 1657.19 |
| | Median | 282.95 | 8.36 | 6.68 | 1796.62 |
| | Min | 85.00 | 0.16 | 3.35 | 443.60 |
| | Max | 1403.01 | 19.03 | 16.98 | 3772.21 |
| (7) | N | 419 | 407 | 428 | 427 |
| | Mean | 349.10 | 11.07 | 7.14 | 1305.34 |
| | Std Dev | 953.12 | 9.26 | 11.69 | 4293.32 |
| | Median | 181.39 | 8.35 | 4.60 | 655.96 |
| | Min | 55.00 | 0.16 | 0.10 | 112.92 |
| | Max | 18000.00 | 56.60 | 152.03 | 65000.00 |
| (8) | N | 11 | 11 | 11 | 11 |
| | Mean | 330.31 | 8.29 | 7.87 | 1402.33 |
| | Std Dev | 382.14 | 5.57 | 4.54 | 1220.54 |
| | Median | 203.63 | 7.49 | 6.58 | 802.08 |
| | Min | 57.67 | 0.16 | 3.23 | 443.60 |
| | Max | 1403.01 | 19.03 | 16.98 | 3772.21 |
| (9) | N | 33 | 33 | 34 | 34 |
| | Mean | 273.59 | 7.06 | 7.52 | 875.12 |
| | Std Dev | 206.06 | 4.83 | 8.83 | 682.36 |
| | Median | 198.65 | 5.89 | 4.35 | 667.69 |
| | Min | 55.00 | 0.16 | 1.41 | 254.65 |
| | Max | 882.40 | 22.47 | 46.42 | 3562.21 |
| (10) | N | 23 | 22 | 23 | 23 |
| | Mean | 428.25 | 8.80 | 6.09 | 1355.30 |
| | Std Dev | 464.20 | 8.99 | 4.59 | 1267.89 |
| | Median | 256.35 | 7.11 | 3.58 | 765.56 |
| | Min | 55.00 | 0.16 | 1.53 | 439.37 |
| | Max | 1882.98 | 43.35 | 16.98 | 4452.08 |
| (11) | N | 396 | 385 | 405 | 404 |
| | Mean | 347.48 | 11.19 | 6.86 | 1144.93 |
| | Std Dev | 975.64 | 9.28 | 9.94 | 3049.29 |
| | Median | 176.03 | 8.54 | 4.62 | 648.25 |
| | Min | 55.00 | 0.16 | 0.10 | 112.92 |
| | Max | 18000.00 | 56.60 | 152.03 | 52268.40 |
| (12) | N | 34 | 33 | 34 | 34 |
| | Mean | 361.91 | 8.69 | 10.68 | 3242.70 |
| | Std Dev | 396.82 | 7.75 | 23.51 | 10985.00 |
| | Median | 245.41 | 7.16 | 5.12 | 822.47 |
| | Min | 55.00 | 0.16 | 1.53 | 439.37 |
| | Max | 1882.98 | 43.35 | 141.20 | 65000.00 |

| Subject | | | | | |
|---|---|---|---|---|---|
| population | | IL-25 | Leptin | sLTBR | MCP-1 |
| (1) | N | 47 | 49 | 49 | 49 |
| | Mean | 422.78 | 19.45 | 2.48 | 55.89 |
| | Std Dev | 769.92 | 27.00 | 1.10 | 129.87 |
| | Median | 137.51 | 10.26 | 2.18 | 29.92 |
| | Min | 50.00 | 0.05 | 1.26 | 3.20 |
| | Max | 3919.56 | 150.00 | 7.23 | 921.86 |
| (2) | N | 48 | 29 | 57 | 29 |
| | Mean | 615.66 | 32.26 | 3.14 | 67.27 |
| | Std Dev | 775.54 | 36.86 | 1.50 | 150.96 |
| | Median | 239.61 | 19.73 | 2.70 | 28.27 |
| | Min | 50.00 | 0.05 | 1.09 | 3.20 |
| | Max | 3174.91 | 150.00 | 9.20 | 721.57 |
| (3) | N | 44 | 34 | 51 | 34 |
| | Mean | 1164.27 | 30.63 | 3.55 | 56.83 |
| | Std Dev | 3415.20 | 33.83 | 2.42 | 120.14 |
| | Median | 222.60 | 20.06 | 3.00 | 33.25 |
| | Min | 50.00 | 0.65 | 1.09 | 3.20 |
| | Max | 22574.24 | 150.00 | 13.07 | 721.57 |
| (4) | N | 327 | 146 | 382 | 146 |
| | Mean | 610.02 | 38.55 | 2.68 | 66.34 |
| | Std Dev | 1665.99 | 37.61 | 1.73 | 224.29 |
| | Median | 192.24 | 27.63 | 2.18 | 27.37 |
| | Min | 50.00 | 0.05 | 0.69 | 3.20 |
| | Max | 22574.24 | 150.00 | 13.59 | 2200.00 |
| (5) | N | 44 | 27 | 53 | 27 |
| | Mean | 663.52 | 34.19 | 3.02 | 67.89 |
| | Std Dev | 793.24 | 37.51 | 1.26 | 156.52 |
| | Median | 288.83 | 20.78 | 2.67 | 26.13 |
| | Min | 50.00 | 0.05 | 1.09 | 3.20 |
| | Max | 3174.91 | 150.00 | 7.60 | 721.57 |
| (6) | N | 4 | 2 | 4 | 2 |
| | Mean | 89.20 | 6.19 | 4.67 | 58.97 |
| | Std Dev | 26.21 | 0.21 | 3.33 | 31.09 |
| | Median | 100.79 | 6.19 | 3.94 | 58.97 |
| | Min | 50.00 | 6.04 | 1.59 | 36.98 |
| | Max | 105.21 | 6.33 | 9.20 | 80.95 |
| (7) | N | 364 | 171 | 428 | 171 |
| | Mean | 621.11 | 38.06 | 2.73 | 66.86 |
| | Std Dev | 1601.03 | 37.63 | 1.69 | 215.94 |
| | Median | 199.37 | 27.32 | 2.26 | 27.15 |
| | Min | 50.00 | 0.05 | 0.69 | 3.20 |
| | Max | 22574.24 | 150.00 | 13.59 | 2200.00 |
| (8) | N | 11 | 4 | 11 | 4 |
| | Mean | 267.56 | 13.70 | 3.42 | 51.11 |
| | Std Dev | 357.71 | 18.98 | 2.21 | 38.40 |
| | Median | 100.94 | 6.19 | 2.74 | 58.97 |
| | Min | 50.00 | 0.55 | 1.58 | 3.20 |
| | Max | 1214.97 | 41.89 | 9.20 | 83.32 |
| (9) | N | 27 | 17 | 34 | 17 |
| | Mean | 401.09 | 36.72 | 3.03 | 67.33 |
| | Std Dev | 415.22 | 46.35 | 1.34 | 169.33 |
| | Median | 231.26 | 11.60 | 2.62 | 28.27 |
| | Min | 50.00 | 0.05 | 1.43 | 3.20 |
| | Max | 1599.46 | 150.00 | 7.60 | 721.57 |
| (10) | N | 21 | 12 | 23 | 12 |
| | Mean | 891.54 | 25.94 | 3.30 | 67.19 |
| | Std Dev | 1023.35 | 16.14 | 1.72 | 127.68 |
| | Median | 247.95 | 25.23 | 2.87 | 25.00 |
| | Min | 50.00 | 2.43 | 1.09 | 8.88 |
| | Max | 3174.91 | 48.84 | 9.20 | 466.96 |
| (11) | N | 343 | 157 | 405 | 157 |
| | Mean | 604.83 | 38.98 | 2.70 | 60.33 |
| | Std Dev | 1629.24 | 38.92 | 1.70 | 202.33 |
| | Median | 199.19 | 27.32 | 2.20 | 27.59 |
| | Min | 50.00 | 0.05 | 0.69 | 3.20 |
| | Max | 22574.24 | 150.00 | 13.59 | 2200.00 |
| (12) | N | 32 | 18 | 34 | 18 |
| | Mean | 674.13 | 24.62 | 3.29 | 120.27 |
| | Std Dev | 900.67 | 16.30 | 1.71 | 295.90 |
| | Median | 175.96 | 25.83 | 2.81 | 25.00 |
| | Min | 50.00 | 0.05 | 1.09 | 3.20 |
| | Max | 3174.91 | 48.84 | 9.20 | 1227.66 |

| Subject | | | | | |
|---|---|---|---|---|---|
| population | | MMP9 | MPO | MYO | NDKA |
| (1) | N | 43 | 49 | 47 | |
| | Mean | 422.30 | 45.77 | 88.56 | 13.10 |
| | Std Dev | 318.28 | 63.97 | 77.54 | 8.28 |
| | Median | 324.10 | 24.81 | 69.47 | 9.81 |
| | Min | 205.48 | 6.20 | 26.18 | 3.58 |
| | Max | 2240.36 | 377.43 | 527.00 | 45.09 |
| (2) | N | 28 | 57 | 51 | 57 |
| | Mean | 1916.86 | 91.21 | 88.80 | 36.15 |
| | Std Dev | 1515.17 | 81.74 | 80.64 | 25.71 |
| | Median | 1449.29 | 68.39 | 68.31 | 30.49 |
| | Min | 264.76 | 2.00 | 14.48 | 2.10 |
| | Max | 6800.00 | 376.79 | 396.39 | 122.12 |
| (3) | N | 32 | 51 | 39 | 51 |
| | Mean | 1710.70 | 71.50 | 113.84 | 40.47 |
| | Std Dev | 1339.09 | 66.70 | 129.52 | 45.68 |
| | Median | 1425.36 | 50.02 | 70.59 | 30.40 |
| | Min | 264.76 | 2.00 | 14.48 | 2.10 |
| | Max | 6800.00 | 376.79 | 527.00 | 300.00 |
| (4) | N | 141 | 382 | 271 | 378 |
| | Mean | 1651.67 | 54.28 | 93.80 | 56.50 |
| | Std Dev | 1442.38 | 183.45 | 98.21 | 54.30 |
| | Median | 1253.73 | 25.80 | 61.41 | 39.80 |
| | Min | 225.41 | 2.00 | 0.79 | 2.10 |
| | Max | 6800.00 | 2000.00 | 527.00 | 300.00 |
| (5) | N | 26 | 53 | 48 | 53 |
| | Mean | 1795.72 | 82.86 | 89.44 | 35.24 |
| | Std Dev | 1382.38 | 70.32 | 82.62 | 26.17 |
| | Median | 1449.29 | 67.22 | 66.32 | 30.40 |
| | Min | 264.76 | 2.00 | 14.48 | 2.10 |
| | Max | 6800.00 | 376.79 | 396.39 | 122.12 |
| (6) | N | 2 | 4 | 3 | 4 |
| | Mean | 3491.73 | 201.79 | 78.46 | 48.20 |
| | Std Dev | 2978.12 | 146.57 | 44.68 | 16.30 |
| | Median | 3491.73 | 211.94 | 88.08 | 50.57 |
| | Min | 1385.88 | 39.16 | 29.75 | 26.18 |
| | Max | 5597.57 | 344.10 | 117.55 | 65.48 |
| (7) | N | 165 | 428 | 312 | 424 |
| | Mean | 1646.81 | 58.07 | 92.95 | 53.87 |
| | Std Dev | 1380.85 | 175.22 | 96.40 | 52.52 |
| | Median | 1259.89 | 28.29 | 61.64 | 38.82 |
| | Min | 225.41 | 2.00 | 0.79 | 2.10 |
| | Max | 6800.00 | 2000.00 | 527.00 | 300.00 |
| (8) | N | 4 | 11 | 10 | 11 |
| | Mean | 3708.44 | 98.33 | 94.92 | 52.39 |
| | Std Dev | 2920.41 | 116.88 | 66.76 | 15.80 |
| | Median | 3491.73 | 42.81 | 86.69 | 50.96 |
| | Min | 1050.32 | 13.18 | 17.20 | 26.18 |
| | Max | 6800.00 | 344.10 | 217.73 | 77.00 |
| (9) | N | 17 | 34 | 30 | 34 |
| | Mean | 1807.72 | 75.85 | 95.32 | 32.98 |
| | Std Dev | 1536.89 | 63.36 | 81.90 | 24.79 |
| | Median | 1433.74 | 55.77 | 73.98 | 29.81 |
| | Min | 264.76 | 2.00 | 14.48 | 2.10 |
| | Max | 6800.00 | 274.45 | 396.39 | 122.12 |
| (10) | N | 11 | 23 | 21 | 23 |
| | Mean | 2085.54 | 113.91 | 79.48 | 40.83 |
| | Std Dev | 1538.75 | 100.41 | 79.86 | 26.88 |
| | Median | 1617.38 | 89.33 | 54.85 | 31.43 |
| | Min | 592.65 | 2.00 | 15.99 | 12.96 |
| | Max | 5597.57 | 376.79 | 386.94 | 103.79 |
| (11) | N | 152 | 405 | 290 | 401 |
| | Mean | 1569.05 | 56.18 | 92.18 | 54.48 |
| | Std Dev | 1284.60 | 179.06 | 94.27 | 53.46 |
| | Median | 1244.08 | 27.04 | 61.64 | 39.19 |
| | Min | 225.41 | 2.00 | 0.79 | 2.10 |
| | Max | 6800.00 | 2000.00 | 527.00 | 300.00 |
| (12) | N | 17 | 34 | 32 | 34 |
| | Mean | 2827.21 | 93.59 | 100.53 | 46.17 |
| | Std Dev | 2257.08 | 89.85 | 107.74 | 26.96 |
| | Median | 1741.88 | 69.06 | 68.78 | 35.39 |
| | Min | 592.65 | 2.00 | 15.99 | 12.96 |
| | Max | 6800.00 | 376.79 | 527.00 | 114.37 |

| Subject | | | | | |
|---|---|---|---|---|---|
| population | | Neuropilin-2 | NGAL | PLGF-1 | PLGF-1/2 |
| (1) | N | 47 | 49 | 49 | 49 |
| | Mean | 353.04 | 314.38 | 217.10 | 49.41 |
| | Std Dev | 73.61 | 114.35 | 721.89 | 61.52 |
| | Median | 343.46 | 299.74 | 18.31 | 28.43 |
| | Min | 217.88 | 55.00 | 15.00 | 17.00 |
| | Max | 577.67 | 698.26 | 3986.54 | 294.92 |
| (2) | N | 54 | 29 | 57 | 57 |
| | Mean | 453.96 | 215.34 | 119.71 | 90.82 |
| | Std Dev | 104.29 | 86.50 | 171.87 | 79.79 |
| | Median | 447.69 | 235.68 | 59.75 | 64.93 |
| | Min | 251.56 | 55.00 | 15.00 | 17.00 |
| | Max | 826.64 | 358.48 | 885.90 | 416.58 |
| (3) | N | 48 | 34 | 51 | 51 |
| | Mean | 451.25 | 217.82 | 161.76 | 116.58 |
| | Std Dev | 108.15 | 83.52 | 364.21 | 218.93 |
| | Median | 454.33 | 230.92 | 33.30 | 60.41 |
| | Min | 251.56 | 55.00 | 15.00 | 17.00 |
| | Max | 826.64 | 386.41 | 2289.05 | 1422.99 |
| (4) | N | 355 | 146 | 382 | 382 |
| | Mean | 454.31 | 228.52 | 104.45 | 95.39 |
| | Std Dev | 127.74 | 95.97 | 248.91 | 216.12 |
| | Median | 441.13 | 225.90 | 23.23 | 48.83 |
| | Min | 2.80 | 55.00 | 15.00 | 17.00 |
| | Max | 1016.30 | 587.10 | 2289.05 | 3350.16 |
| (5) | N | 50 | 27 | 53 | 53 |
| | Mean | 455.64 | 209.35 | 107.52 | 84.53 |
| | Std Dev | 104.76 | 84.97 | 140.69 | 66.53 |
| | Median | 448.18 | 235.68 | 59.75 | 64.93 |
| | Min | 251.56 | 55.00 | 15.00 | 17.00 |
| | Max | 826.64 | 318.43 | 714.42 | 317.06 |
| (6) | N | 4 | 2 | 4 | 4 |
| | Mean | 432.96 | 296.12 | 281.33 | 174.25 |
| | Std Dev | 110.80 | 88.18 | 413.34 | 179.30 |
| | Median | 421.56 | 296.12 | 112.21 | 128.99 |
| | Min | 309.88 | 233.77 | 15.00 | 22.45 |
| | Max | 578.82 | 358.48 | 885.90 | 416.58 |
| (7) | N | 400 | 171 | 428 | 428 |
| | Mean | 453.48 | 226.54 | 104.43 | 93.78 |
| | Std Dev | 125.30 | 94.00 | 238.30 | 204.33 |
| | Median | 441.15 | 228.07 | 26.13 | 51.45 |
| | Min | 2.80 | 55.00 | 15.00 | 17.00 |
| | Max | 1016.30 | 587.10 | 2289.05 | 3350.16 |
| (8) | N | 9 | 4 | 11 | 11 |
| | Mean | 489.10 | 217.61 | 184.46 | 134.41 |
| | Std Dev | 98.16 | 124.60 | 309.02 | 175.13 |
| | Median | 473.92 | 228.49 | 15.00 | 45.78 |
| | Min | 309.88 | 55.00 | 15.00 | 19.95 |
| | Max | 627.63 | 358.48 | 885.90 | 522.67 |
| (9) | N | 32 | 17 | 34 | 34 |
| | Mean | 442.10 | 208.23 | 94.61 | 80.58 |
| | Std Dev | 94.28 | 86.81 | 140.60 | 67.93 |
| | Median | 446.54 | 230.06 | 54.25 | 61.58 |
| | Min | 251.56 | 55.00 | 15.00 | 17.00 |
| | Max | 599.30 | 318.43 | 714.42 | 317.06 |
| (10) | N | 22 | 12 | 23 | 23 |
| | Mean | 471.20 | 225.41 | 156.83 | 105.96 |
| | Std Dev | 117.48 | 88.86 | 207.66 | 94.25 |
| | Median | 448.18 | 247.62 | 62.03 | 81.37 |
| | Min | 309.88 | 55.00 | 15.00 | 19.40 |
| | Max | 826.64 | 358.48 | 885.90 | 416.58 |
| (11) | N | 379 | 157 | 405 | 405 |
| | Mean | 452.51 | 227.52 | 103.37 | 93.91 |
| | Std Dev | 125.79 | 93.65 | 242.91 | 209.51 |
| | Median | 440.12 | 228.07 | 25.40 | 50.68 |
| | Min | 2.80 | 55.00 | 15.00 | 17.00 |
| | Max | 1016.30 | 587.10 | 2289.05 | 3350.16 |
| (12) | N | 30 | 18 | 34 | 34 |
| | Mean | 476.38 | 216.08 | 142.85 | 105.38 |
| | Std Dev | 110.73 | 102.53 | 204.85 | 112.19 |
| | Median | 464.52 | 229.86 | 59.67 | 67.41 |
| | Min | 309.88 | 55.00 | 15.00 | 19.40 |
| | Max | 826.64 | 412.92 | 885.90 | 522.67 |

| Subject | | Activated | Total Protein C | | |
|---|---|---|---|---|---|
| population | | Protein C | Latent + Active | PSAP-A | PSAP-B |
| (1) | N | 49 | 49 | 49 | 49 |
| | Mean | 0.88 | 36488.69 | 200.40 | 3786.41 |
| | Std Dev | 1.25 | 17287.58 | 522.50 | 2842.33 |
| | Median | 0.44 | 50000.00 | 47.89 | 2973.37 |
| | Min | 0.40 | 1787.82 | 19.93 | 729.35 |
| | Max | 7.00 | 50000.00 | 2727.78 | 12003.98 |
| (2) | N | 57 | 57 | 57 | 57 |
| | Mean | 1.30 | 14109.16 | 131.25 | 4205.15 |
| | Std Dev | 1.07 | 12524.76 | 507.96 | 4378.25 |
| | Median | 0.83 | 9816.18 | 53.86 | 2620.34 |
| | Min | 0.40 | 1700.00 | 1.25 | 24.45 |
| | Max | 4.37 | 50000.00 | 3868.15 | 21760.63 |
| (3) | N | 51 | 51 | 51 | 51 |
| | Mean | 1.78 | 12957.06 | 65.31 | 3838.40 |
| | Std Dev | 3.75 | 13999.99 | 68.50 | 3642.31 |
| | Median | 0.65 | 6099.43 | 54.93 | 2620.34 |
| | Min | 0.40 | 1700.00 | 1.25 | 24.45 |
| | Max | 20.07 | 50000.00 | 504.65 | 18220.04 |
| (4) | N | 382 | 382 | 382 | 382 |
| | Mean | 1.32 | 16589.97 | 59.97 | 3938.16 |
| | Std Dev | 2.32 | 15492.04 | 57.63 | 8246.81 |
| | Median | 0.56 | 10008.37 | 45.50 | 1984.25 |
| | Min | 0.40 | 1700.00 | 1.25 | 10.00 |
| | Max | 21.13 | 50000.00 | 870.04 | 125000.00 |
| (5) | N | 53 | 53 | 53 | 53 |
| | Mean | 1.28 | 14204.50 | 134.56 | 3686.73 |
| | Std Dev | 1.07 | 12919.83 | 526.98 | 3462.28 |
| | Median | 0.83 | 9188.60 | 51.45 | 2484.50 |
| | Min | 0.40 | 1700.00 | 1.25 | 24.45 |
| | Max | 4.37 | 50000.00 | 3868.15 | 18220.04 |
| (6) | N | 4 | 4 | 4 | 4 |
| | Mean | 1.53 | 12845.96 | 87.45 | 11074.28 |
| | Std Dev | 1.22 | 5711.97 | 8.02 | 9076.30 |
| | Median | 1.49 | 13331.46 | 90.34 | 9619.32 |
| | Min | 0.40 | 5801.26 | 76.05 | 3297.84 |
| | Max | 2.75 | 18919.68 | 93.09 | 21760.63 |
| (7) | N | 428 | 428 | 428 | 428 |
| | Mean | 1.31 | 16352.96 | 69.10 | 3928.08 |
| | Std Dev | 2.20 | 15190.64 | 193.31 | 7878.82 |
| | Median | 0.64 | 9874.74 | 46.04 | 2053.81 |
| | Min | 0.40 | 1700.00 | 1.25 | 10.00 |
| | Max | 21.13 | 50000.00 | 3868.15 | 125000.00 |
| (8) | N | 11 | 11 | 11 | 11 |
| | Mean | 1.58 | 12956.81 | 74.28 | 5713.73 |
| | Std Dev | 2.09 | 13692.88 | 32.04 | 6694.53 |
| | Median | 0.58 | 10119.62 | 71.69 | 3382.60 |
| | Min | 0.40 | 1700.00 | 37.52 | 10.00 |
| | Max | 7.37 | 50000.00 | 149.87 | 21760.63 |
| (9) | N | 34 | 34 | 34 | 34 |
| | Mean | 1.06 | 13678.75 | 67.74 | 3473.44 |
| | Std Dev | 0.83 | 12102.80 | 81.43 | 2846.49 |
| | Median | 0.73 | 9502.39 | 51.02 | 2672.19 |
| | Min | 0.40 | 1700.00 | 1.25 | 24.45 |
| | Max | 3.52 | 50000.00 | 504.65 | 13137.25 |
| (10) | N | 23 | 23 | 23 | 23 |
| | Mean | 1.65 | 14745.42 | 225.14 | 5286.81 |
| | Std Dev | 1.29 | 13374.53 | 794.61 | 5881.29 |
| | Median | 1.19 | 10792.51 | 62.42 | 2484.50 |
| | Min | 0.40 | 1700.00 | 1.25 | 197.84 |
| | Max | 4.37 | 50000.00 | 3868.15 | 21760.63 |
| (11) | N | 405 | 405 | 405 | 405 |
| | Mean | 1.30 | 16477.57 | 60.38 | 3913.25 |
| | Std Dev | 2.24 | 15290.90 | 60.36 | 8036.80 |
| | Median | 0.58 | 9865.43 | 45.70 | 2041.83 |
| | Min | 0.40 | 1700.00 | 1.25 | 10.00 |
| | Max | 21.13 | 50000.00 | 870.04 | 125000.00 |
| (12) | N | 34 | 34 | 34 | 34 |
| | Mean | 1.53 | 13769.86 | 174.64 | 4682.43 |
| | Std Dev | 1.55 | 13304.56 | 653.32 | 5151.47 |
| | Median | 0.79 | 10024.30 | 61.03 | 2719.12 |
| | Min | 0.40 | 1700.00 | 1.25 | 10.00 |
| | Max | 7.37 | 50000.00 | 3868.15 | 21760.63 |

| Subject | | | | | |
|---|---|---|---|---|---|
| population | | PSAP-C | PSAP-D | sRAGE | sPECAM-1 |
| (1) | N | 47 | 49 | 49 | 48 |
| | Mean | 0.61 | 3.51 | 0.86 | 12.31 |
| | Std Dev | 0.94 | 2.72 | 0.53 | 2.98 |
| | Median | 0.30 | 3.13 | 0.72 | 11.89 |
| | Min | 0.30 | 0.50 | 0.13 | 8.56 |
| | Max | 4.58 | 10.72 | 3.05 | 23.57 |
| (2) | N | 55 | 57 | 57 | 25 |
| | Mean | 2.45 | 3.95 | 1.20 | 12.99 |
| | Std Dev | 9.88 | 3.44 | 0.83 | 2.13 |
| | Median | 0.30 | 2.89 | 0.98 | 12.56 |
| | Min | 0.30 | 0.50 | 0.07 | 9.72 |
| | Max | 72.60 | 15.00 | 4.09 | 18.36 |
| (3) | N | 48 | 51 | 51 | 31 |
| | Mean | 2.72 | 4.00 | 1.19 | 13.72 |
| | Std Dev | 10.71 | 3.66 | 0.83 | 2.80 |
| | Median | 0.30 | 2.67 | 0.96 | 13.53 |
| | Min | 0.30 | 0.50 | 0.07 | 9.11 |
| | Max | 72.60 | 15.00 | 3.70 | 24.69 |
| (4) | N | 365 | 382 | 382 | 130 |
| | Mean | 0.98 | 3.58 | 1.09 | 13.87 |
| | Std Dev | 2.27 | 3.02 | 0.69 | 5.10 |
| | Median | 0.30 | 2.96 | 0.93 | 12.88 |
| | Min | 0.30 | 0.50 | 0.07 | 8.13 |
| | Max | 19.99 | 22.21 | 5.54 | 53.94 |
| (5) | N | 51 | 53 | 53 | 23 |
| | Mean | 2.34 | 3.91 | 1.23 | 12.79 |
| | Std Dev | 10.14 | 3.54 | 0.84 | 1.89 |
| | Median | 0.30 | 2.32 | 1.02 | 12.56 |
| | Min | 0.30 | 0.50 | 0.07 | 9.72 |
| | Max | 72.60 | 15.00 | 4.09 | 16.90 |
| (6) | N | 4 | 4 | 4 | 2 |
| | Mean | 3.79 | 4.49 | 0.82 | 15.38 |
| | Std Dev | 6.29 | 1.74 | 0.67 | 4.21 |
| | Median | 0.83 | 4.00 | 0.65 | 15.38 |
| | Min | 0.30 | 3.03 | 0.21 | 12.41 |
| | Max | 13.20 | 6.93 | 1.77 | 18.36 |
| (7) | N | 409 | 428 | 428 | 151 |
| | Mean | 1.15 | 3.62 | 1.10 | 13.61 |
| | Std Dev | 4.17 | 3.10 | 0.71 | 4.72 |
| | Median | 0.30 | 2.90 | 0.94 | 12.68 |
| | Min | 0.30 | 0.50 | 0.07 | 8.13 |
| | Max | 72.60 | 22.21 | 5.54 | 53.94 |
| (8) | N | 11 | 11 | 11 | 4 |
| | Mean | 2.16 | 3.89 | 0.98 | 17.92 |
| | Std Dev | 3.97 | 2.14 | 0.83 | 4.33 |
| | Median | 0.30 | 3.49 | 0.76 | 18.15 |
| | Min | 0.30 | 0.85 | 0.07 | 12.41 |
| | Max | 13.20 | 6.93 | 2.62 | 22.99 |
| (9) | N | 33 | 34 | 34 | 15 |
| | Mean | 0.61 | 3.89 | 1.24 | 12.83 |
| | Std Dev | 0.55 | 3.60 | 0.80 | 1.37 |
| | Median | 0.30 | 2.49 | 1.06 | 12.56 |
| | Min | 0.30 | 0.50 | 0.07 | 10.09 |
| | Max | 2.41 | 13.75 | 3.46 | 15.45 |
| (10) | N | 22 | 23 | 23 | 10 |
| | Mean | 5.21 | 4.05 | 1.14 | 13.24 |
| | Std Dev | 15.40 | 3.26 | 0.89 | 3.00 |
| | Median | 0.38 | 3.47 | 0.84 | 12.65 |
| | Min | 0.30 | 0.63 | 0.15 | 9.72 |
| | Max | 72.60 | 15.00 | 4.09 | 18.36 |
| (11) | N | 387 | 405 | 405 | 140 |
| | Mean | 0.95 | 3.62 | 1.10 | 13.60 |
| | Std Dev | 2.20 | 3.09 | 0.69 | 4.81 |
| | Median | 0.30 | 2.91 | 0.93 | 12.64 |
| | Min | 0.30 | 0.50 | 0.07 | 8.13 |
| | Max | 19.99 | 22.21 | 5.54 | 53.94 |
| (12) | N | 33 | 34 | 34 | 15 |
| | Mean | 3.86 | 3.75 | 1.16 | 14.93 |
| | Std Dev | 12.66 | 2.93 | 0.90 | 4.07 |
| | Median | 0.30 | 3.11 | 0.90 | 14.46 |
| | Min | 0.30 | 0.52 | 0.07 | 9.72 |
| | Max | 72.60 | 15.00 | 4.09 | 22.99 |

| Subject | | | | | |
|---|---|---|---|---|---|
| population | | Spectrin 120 | Spectrin 145 | TIE-2 | Tissue Factor |
| (1) | N | 49 | 46 | 49 | 49 |
| | Mean | 1516.45 | 0.84 | 7.75 | 29.35 |
| | Std Dev | 3293.34 | 1.54 | 14.25 | 63.45 |
| | Median | 203.09 | 0.50 | 4.07 | 4.13 |
| | Min | 125.00 | 0.50 | 1.00 | 3.00 |
| | Max | 17018.08 | 10.56 | 74.18 | 346.11 |
| (2) | N | 29 | 27 | 57 | 57 |
| | Mean | 877.63 | 0.79 | 21.88 | 48.41 |
| | Std Dev | 2714.65 | 0.79 | 91.52 | 64.74 |
| | Median | 125.00 | 0.50 | 5.03 | 20.37 |
| | Min | 125.00 | 0.50 | 1.00 | 3.00 |
| | Max | 14721.02 | 4.04 | 600.00 | 305.11 |
| (3) | N | 34 | 33 | 51 | 51 |
| | Mean | 981.61 | 1.64 | 17.09 | 68.05 |
| | Std Dev | 2722.01 | 3.43 | 83.28 | 130.50 |
| | Median | 125.00 | 0.50 | 5.11 | 14.87 |
| | Min | 125.00 | 0.50 | 2.69 | 3.00 |
| | Max | 14721.02 | 18.56 | 600.00 | 712.87 |
| (4) | N | 146 | 146 | 382 | 382 |
| | Mean | 392.66 | 0.96 | 5.25 | 50.11 |
| | Std Dev | 767.57 | 1.85 | 2.48 | 119.78 |
| | Median | 125.00 | 0.50 | 4.95 | 11.00 |
| | Min | 125.00 | 0.50 | 1.00 | 3.00 |
| | Max | 6627.97 | 18.56 | 36.11 | 1163.54 |
| (5) | N | 27 | 25 | 53 | 53 |
| | Mean | 927.18 | 0.74 | 23.17 | 47.46 |
| | Std Dev | 2810.46 | 0.77 | 94.85 | 64.84 |
| | Median | 125.00 | 0.50 | 5.13 | 20.37 |
| | Min | 125.00 | 0.50 | 1.00 | 3.00 |
| | Max | 14721.02 | 4.04 | 600.00 | 305.11 |
| (6) | N | 2 | 2 | 4 | 4 |
| | Mean | 208.79 | 1.34 | 4.85 | 60.99 |
| | Std Dev | 118.49 | 1.18 | 0.85 | 71.66 |
| | Median | 208.79 | 1.34 | 4.56 | 43.83 |
| | Min | 125.00 | 0.50 | 4.24 | 3.00 |
| | Max | 292.58 | 2.17 | 6.05 | 153.32 |
| (7) | N | 171 | 169 | 428 | 428 |
| | Mean | 479.64 | 0.93 | 7.46 | 49.42 |
| | Std Dev | 1322.02 | 1.74 | 33.70 | 113.97 |
| | Median | 125.00 | 0.50 | 4.99 | 11.82 |
| | Min | 125.00 | 0.50 | 1.00 | 3.00 |
| | Max | 14721.02 | 18.56 | 600.00 | 1163.54 |
| (8) | N | 4 | 4 | 11 | 11 |
| | Mean | 190.29 | 1.02 | 5.53 | 67.81 |
| | Std Dev | 81.22 | 0.79 | 2.27 | 123.09 |
| | Median | 171.80 | 0.69 | 4.86 | 6.52 |
| | Min | 125.00 | 0.50 | 3.62 | 3.00 |
| | Max | 292.58 | 2.17 | 11.23 | 410.05 |
| (9) | N | 17 | 16 | 34 | 34 |
| | Mean | 1106.34 | 0.66 | 22.66 | 36.20 |
| | Std Dev | 3515.89 | 0.42 | 102.04 | 53.76 |
| | Median | 125.00 | 0.50 | 5.14 | 17.09 |
| | Min | 125.00 | 0.50 | 1.00 | 3.00 |
| | Max | 14721.02 | 1.96 | 600.00 | 261.09 |
| (10) | N | 12 | 11 | 23 | 23 |
| | Mean | 553.63 | 0.97 | 20.73 | 66.46 |
| | Std Dev | 763.28 | 1.13 | 75.50 | 75.90 |
| | Median | 223.50 | 0.50 | 4.91 | 35.67 |
| | Min | 125.00 | 0.50 | 2.69 | 3.00 |
| | Max | 2732.40 | 4.04 | 367.00 | 305.11 |
| (11) | N | 157 | 156 | 405 | 405 |
| | Mean | 473.37 | 0.93 | 6.71 | 48.74 |
| | Std Dev | 1363.68 | 1.79 | 29.66 | 115.87 |
| | Median | 125.00 | 0.50 | 5.00 | 11.19 |
| | Min | 125.00 | 0.50 | 1.00 | 3.00 |
| | Max | 14721.02 | 18.56 | 600.00 | 1163.54 |
| (12) | N | 18 | 17 | 34 | 34 |
| | Mean | 470.00 | 0.98 | 15.80 | 63.50 |
| | Std Dev | 658.05 | 1.06 | 62.08 | 90.47 |
| | Median | 156.92 | 0.50 | 4.89 | 26.62 |
| | Min | 125.00 | 0.50 | 2.69 | 3.00 |
| | Max | 2732.40 | 4.04 | 367.00 | 410.05 |

| Subject | | | | | |
|---|---|---|---|---|---|
| population | | sTNF-R1a | sTNFRSF7 | TNFsR14 | cTNI |
| (1) | N | 49 | 49 | 49 | 34 |
| | Mean | 11.43 | 7.25 | 241.12 | 0.05 |
| | Std Dev | 6.14 | 13.49 | 323.29 | 0.03 |
| | Median | 10.12 | 3.30 | 118.06 | 0.05 |
| | Min | 5.69 | 0.69 | 85.00 | 0.05 |
| | Max | 46.29 | 69.98 | 1738.87 | 0.20 |
| (2) | N | 57 | 57 | 57 | 51 |
| | Mean | 17.11 | 6.23 | 335.90 | 0.06 |
| | Std Dev | 9.23 | 5.74 | 325.12 | 0.04 |
| | Median | 14.06 | 4.42 | 248.92 | 0.05 |
| | Min | 2.39 | 0.93 | 85.00 | 0.05 |
| | Max | 55.70 | 28.40 | 1530.34 | 0.25 |
| (3) | N | 51 | 51 | 51 | 39 |
| | Mean | 17.49 | 6.10 | 649.11 | 0.06 |
| | Std Dev | 10.07 | 5.17 | 1794.41 | 0.04 |
| | Median | 14.89 | 4.90 | 179.30 | 0.05 |
| | Min | 2.39 | 1.31 | 85.00 | 0.05 |
| | Max | 60.15 | 32.14 | 10643.29 | 0.25 |
| (4) | N | 382 | 382 | 380 | 271 |
| | Mean | 15.17 | 5.99 | 343.28 | 0.14 |
| | Std Dev | 10.30 | 7.35 | 788.29 | 0.99 |
| | Median | 12.38 | 3.91 | 145.57 | 0.05 |
| | Min | 4.98 | 0.82 | 85.00 | 0.05 |
| | Max | 121.93 | 96.13 | 10643.29 | 14.34 |
| (5) | N | 53 | 53 | 53 | 48 |
| | Mean | 16.30 | 5.80 | 316.83 | 0.06 |
| | Std Dev | 7.70 | 5.05 | 302.06 | 0.04 |
| | Median | 13.25 | 4.38 | 236.94 | 0.05 |
| | Min | 2.39 | 0.93 | 85.00 | 0.05 |
| | Max | 42.70 | 22.69 | 1530.34 | 0.25 |
| (6) | N | 4 | 4 | 4 | 3 |
| | Mean | 27.83 | 11.92 | 588.67 | 0.06 |
| | Std Dev | 19.99 | 11.22 | 547.67 | 0.01 |
| | Median | 22.06 | 7.80 | 481.45 | 0.05 |
| | Min | 11.51 | 3.67 | 85.00 | 0.05 |
| | Max | 55.70 | 28.40 | 1306.80 | 0.07 |
| (7) | N | 428 | 428 | 426 | 312 |
| | Mean | 15.28 | 5.98 | 338.03 | 0.13 |
| | Std Dev | 10.06 | 7.16 | 746.28 | 0.93 |
| | Median | 12.67 | 3.99 | 152.86 | 0.05 |
| | Min | 2.39 | 0.82 | 85.00 | 0.05 |
| | Max | 121.93 | 96.13 | 10643.29 | 14.34 |
| (8) | N | 11 | 11 | 11 | 10 |
| | Mean | 20.85 | 7.73 | 508.29 | 0.05 |
| | Std Dev | 13.42 | 7.23 | 665.46 | 0.01 |
| | Median | 15.40 | 6.34 | 185.34 | 0.05 |
| | Min | 11.51 | 3.42 | 85.00 | 0.05 |
| | Max | 55.70 | 28.40 | 2164.12 | 0.07 |
| (9) | N | 34 | 34 | 34 | 30 |
| | Mean | 16.30 | 5.81 | 278.21 | 0.06 |
| | Std Dev | 8.32 | 5.26 | 278.87 | 0.02 |
| | Median | 13.09 | 4.24 | 185.16 | 0.05 |
| | Min | 2.39 | 0.93 | 85.00 | 0.05 |
| | Max | 42.70 | 22.69 | 1245.37 | 0.17 |
| (10) | N | 23 | 23 | 23 | 21 |
| | Mean | 18.31 | 6.84 | 421.18 | 0.08 |
| | Std Dev | 10.52 | 6.45 | 373.71 | 0.06 |
| | Median | 14.33 | 4.56 | 343.15 | 0.05 |
| | Min | 8.83 | 1.68 | 85.00 | 0.05 |
| | Max | 55.70 | 28.40 | 1530.34 | 0.25 |
| (11) | N | 405 | 405 | 403 | 290 |
| | Mean | 15.06 | 5.96 | 336.32 | 0.10 |
| | Std Dev | 9.89 | 7.27 | 763.56 | 0.84 |
| | Median | 12.36 | 3.92 | 145.27 | 0.05 |
| | Min | 2.39 | 0.82 | 85.00 | 0.05 |
| | Max | 121.93 | 96.13 | 10643.29 | 14.34 |
| (12) | N | 34 | 34 | 34 | 32 |
| | Mean | 19.74 | 6.80 | 413.40 | 0.32 |
| | Std Dev | 12.48 | 5.68 | 456.46 | 1.42 |
| | Median | 15.00 | 4.75 | 272.78 | 0.05 |
| | Min | 8.83 | 1.68 | 85.00 | 0.05 |
| | Max | 65.23 | 28.40 | 2164.12 | 8.10 |

| Subject | | | | | |
|---|---|---|---|---|---|
| population | | TpP | UFDP1H | UPA | VCAM-1 |
| (1) | N | 42 | 48 | 49 | 46 |
| | Mean | 0.18 | 0.36 | 1.04 | 2396.97 |
| | Std Dev | 0.25 | 0.46 | 0.29 | 1201.34 |
| | Median | 0.09 | 0.20 | 1.05 | 2206.19 |
| | Min | 0.04 | 0.20 | 0.04 | 700.00 |
| | Max | 1.55 | 2.89 | 2.12 | 5771.97 |
| (2) | N | 24 | 29 | 57 | 53 |
| | Mean | 0.28 | 2.38 | 0.97 | 1630.52 |
| | Std Dev | 0.34 | 3.74 | 0.46 | 599.35 |
| | Median | 0.12 | 1.20 | 0.87 | 1555.51 |
| | Min | 0.05 | 0.20 | 0.04 | 700.00 |
| | Max | 1.33 | 17.58 | 2.71 | 3304.19 |
| (3) | N | 30 | 34 | 51 | 49 |
| | Mean | 0.42 | 2.17 | 1.03 | 1492.52 |
| | Std Dev | 0.70 | 3.43 | 0.52 | 552.41 |
| | Median | 0.12 | 1.11 | 0.88 | 1427.17 |
| | Min | 0.04 | 0.20 | 0.04 | 700.00 |
| | Max | 3.37 | 17.58 | 3.45 | 3304.19 |
| (4) | N | 128 | 146 | 378 | 370 |
| | Mean | 0.24 | 3.89 | 0.95 | 1652.41 |
| | Std Dev | 0.45 | 4.84 | 0.54 | 548.15 |
| | Median | 0.12 | 1.73 | 0.89 | 1620.81 |
| | Min | 0.03 | 0.20 | 0.04 | 700.00 |
| | Max | 3.37 | 23.56 | 5.61 | 3407.24 |
| (5) | N | 22 | 27 | 53 | 49 |
| | Mean | 0.29 | 2.29 | 0.91 | 1612.31 |
| | Std Dev | 0.35 | 3.87 | 0.36 | 585.42 |
| | Median | 0.12 | 0.89 | 0.87 | 1555.51 |
| | Min | 0.05 | 0.20 | 0.04 | 700.00 |
| | Max | 1.33 | 17.58 | 2.13 | 3304.19 |
| (6) | N | 2 | 2 | 4 | 4 |
| | Mean | 0.14 | 3.50 | 1.71 | 1853.56 |
| | Std Dev | 0.12 | 0.74 | 0.95 | 819.24 |
| | Median | 0.14 | 3.50 | 1.68 | 1783.19 |
| | Min | 0.05 | 2.98 | 0.77 | 1040.12 |
| | Max | 0.22 | 4.02 | 2.71 | 2807.74 |
| (7) | N | 149 | 171 | 424 | 413 |
| | Mean | 0.25 | 3.67 | 0.95 | 1648.71 |
| | Std Dev | 0.44 | 4.74 | 0.53 | 551.66 |
| | Median | 0.12 | 1.56 | 0.88 | 1598.06 |
| | Min | 0.03 | 0.20 | 0.04 | 700.00 |
| | Max | 3.37 | 23.56 | 5.61 | 3407.24 |
| (8) | N | 3 | 4 | 11 | 10 |
| | Mean | 0.14 | 2.28 | 1.13 | 1689.20 |
| | Std Dev | 0.08 | 1.49 | 0.77 | 681.15 |
| | Median | 0.14 | 2.15 | 0.99 | 1690.42 |
| | Min | 0.05 | 0.79 | 0.04 | 700.00 |
| | Max | 0.22 | 4.02 | 2.71 | 2807.74 |
| (9) | N | 14 | 17 | 34 | 31 |
| | Mean | 0.28 | 1.99 | 0.90 | 1559.84 |
| | Std Dev | 0.37 | 2.90 | 0.37 | 643.71 |
| | Median | 0.12 | 0.76 | 0.87 | 1504.60 |
| | Min | 0.05 | 0.20 | 0.04 | 700.00 |
| | Max | 1.33 | 10.51 | 2.13 | 3304.19 |
| (10) | N | 10 | 12 | 23 | 22 |
| | Mean | 0.27 | 2.92 | 1.07 | 1730.12 |
| | Std Dev | 0.30 | 4.79 | 0.56 | 528.94 |
| | Median | 0.17 | 1.26 | 0.85 | 1636.06 |
| | Min | 0.05 | 0.20 | 0.55 | 1040.12 |
| | Max | 1.01 | 17.58 | 2.71 | 2807.74 |
| (11) | N | 137 | 157 | 401 | 391 |
| | Mean | 0.25 | 3.75 | 0.95 | 1647.80 |
| | Std Dev | 0.45 | 4.77 | 0.53 | 555.92 |
| | Median | 0.12 | 1.63 | 0.88 | 1598.06 |
| | Min | 0.04 | 0.20 | 0.04 | 700.00 |
| | Max | 3.37 | 23.56 | 5.61 | 3407.24 |
| (12) | N | 15 | 18 | 34 | 32 |
| | Mean | 0.23 | 2.64 | 0.97 | 1672.46 |
| | Std Dev | 0.26 | 3.95 | 0.55 | 539.57 |
| | Median | 0.11 | 1.35 | 0.82 | 1627.58 |
| | Min | 0.03 | 0.20 | 0.04 | 700.00 |
| | Max | 1.01 | 17.58 | 2.71 | 2807.74 |

| Subject | | | | | |
|---|---|---|---|---|---|
| population | | VE-Cadherin | VEGF | sVEGF-R1 | sVEGF-R2 |
| (1) | N | 48 | 46 | 42 | 49 |
| | Mean | 64285.33 | 78.43 | 492.64 | 190.09 |
| | Std Dev | 33516.14 | 145.69 | 453.27 | 36.73 |
| | Median | 59630.36 | 25.00 | 374.28 | 197.68 |
| | Min | 90.00 | 25.00 | 150.00 | 22.75 |
| | Max | 167399.00 | 823.08 | 2194.21 | 261.63 |
| (2) | N | 51 | 55 | 48 | 57 |
| | Mean | 54152.84 | 170.16 | 1547.66 | 181.27 |
| | Std Dev | 32910.39 | 219.26 | 2748.89 | 39.09 |
| | Median | 47692.58 | 78.99 | 780.63 | 178.81 |
| | Min | 90.00 | 25.00 | 150.00 | 13.00 |
| | Max | 156948.20 | 1127.77 | 18106.12 | 252.16 |
| (3) | N | 46 | 50 | 45 | 51 |
| | Mean | 60870.66 | 212.80 | 1785.39 | 191.36 |
| | Std Dev | 40277.89 | 412.82 | 3470.31 | 28.61 |
| | Median | 59877.57 | 80.39 | 768.45 | 185.69 |
| | Min | 90.00 | 25.00 | 150.00 | 127.22 |
| | Max | 185000.00 | 2783.22 | 18106.12 | 252.16 |
| (4) | N | 367 | 382 | 314 | 378 |
| | Mean | 60488.55 | 169.85 | 927.49 | 181.26 |
| | Std Dev | 29906.22 | 309.32 | 2203.43 | 40.07 |
| | Median | 55918.84 | 85.74 | 445.60 | 184.26 |
| | Min | 90.00 | 25.00 | 150.00 | 13.00 |
| | Max | 185000.00 | 3502.73 | 21307.45 | 289.16 |
| (5) | N | 48 | 51 | 47 | 53 |
| | Mean | 53675.94 | 154.13 | 1571.22 | 180.42 |
| | Std Dev | 33544.08 | 203.12 | 2773.70 | 39.94 |
| | Median | 47431.11 | 60.35 | 792.80 | 178.81 |
| | Min | 90.00 | 25.00 | 150.00 | 13.00 |
| | Max | 156948.20 | 1127.77 | 18106.12 | 252.16 |
| (6) | N | 3 | 4 | 1 | 4 |
| | Mean | 61783.25 | 374.54 | 440.32 | 192.48 |
| | Std Dev | 23285.81 | 342.99 | #DIV/0! | 26.30 |
| | Median | 59528.50 | 237.60 | 440.32 | 185.02 |
| | Min | 39706.84 | 140.74 | 440.32 | 170.55 |
| | Max | 86114.42 | 882.23 | 440.32 | 229.35 |
| (7) | N | 408 | 426 | 355 | 424 |
| | Mean | 59922.60 | 167.65 | 1019.66 | 181.41 |
| | Std Dev | 30258.26 | 300.23 | 2309.77 | 39.44 |
| | Median | 55192.79 | 82.84 | 469.68 | 183.49 |
| | Min | 90.00 | 25.00 | 150.00 | 13.00 |
| | Max | 185000.00 | 3502.73 | 21307.45 | 289.16 |
| (8) | N | 10 | 11 | 7 | 11 |
| | Mean | 51266.94 | 256.50 | 505.87 | 175.82 |
| | Std Dev | 33217.40 | 255.96 | 252.34 | 56.89 |
| | Median | 54468.96 | 198.79 | 454.70 | 192.89 |
| | Min | 90.00 | 56.61 | 150.00 | 13.00 |
| | Max | 113869.20 | 882.23 | 871.60 | 229.35 |
| (9) | N | 31 | 33 | 30 | 34 |
| | Mean | 52287.68 | 104.75 | 1312.51 | 181.24 |
| | Std Dev | 33827.69 | 116.31 | 3210.82 | 46.48 |
| | Median | 48489.39 | 55.52 | 612.32 | 182.84 |
| | Min | 90.00 | 25.00 | 150.00 | 13.00 |
| | Max | 139346.40 | 514.77 | 18106.12 | 252.16 |
| (10) | N | 20 | 22 | 18 | 23 |
| | Mean | 57043.85 | 268.27 | 1939.58 | 181.31 |
| | Std Dev | 32077.45 | 293.60 | 1744.82 | 25.50 |
| | Median | 47431.11 | 169.77 | 1523.21 | 177.15 |
| | Min | 12241.10 | 25.00 | 150.00 | 140.31 |
| | Max | 156948.20 | 1127.77 | 6089.31 | 244.32 |
| (11) | N | 388 | 404 | 335 | 401 |
| | Mean | 60048.41 | 165.02 | 971.97 | 181.96 |
| | Std Dev | 30135.91 | 301.97 | 2334.59 | 39.26 |
| | Median | 55502.54 | 82.84 | 460.81 | 184.36 |
| | Min | 90.00 | 25.00 | 150.00 | 13.00 |
| | Max | 185000.00 | 3502.73 | 21307.45 | 289.16 |
| (12) | N | 30 | 33 | 27 | 34 |
| | Mean | 55410.26 | 229.54 | 1478.17 | 173.12 |
| | Std Dev | 32818.06 | 260.69 | 1568.99 | 46.68 |
| | Median | 49472.25 | 128.68 | 857.38 | 177.15 |
| | Min | 90.00 | 25.00 | 150.00 | 13.00 |
| | Max | 156948.20 | 1127.77 | 6089.31 | 244.32 |

| Subject | | | |
|---|---|---|---|
| population | | VWF-Integrin | ANP₂₈₋₁₅₁ |
| (1) | N | 49 | 48 |
| | Mean | 25255.69 | 1.82 |
| | Std Dev | 50850.34 | 3.91 |
| | Median | 8457.35 | 0 |
| | Min | 2800.00 | 0 |
| | Max | 205508.10 | 17.75 |
| (2) | N | 57 | 54 |
| | Mean | 33500.97 | 4.02 |
| | Std Dev | 40376.56 | 8.03 |
| | Median | 23488.50 | 0.86 |
| | Min | 2800.00 | 0 |
| | Max | 241140.20 | 46.37 |
| (3) | N | 51 | 51 |
| | Mean | 30202.70 | 5.96 |
| | Std Dev | 32859.12 | 17.90 |
| | Median | 16837.07 | 0.61 |
| | Min | 2800.00 | 0 |
| | Max | 188961.30 | 120 |
| (4) | N | 382 | 369 |
| | Mean | 22265.47 | 3.11 |
| | Std Dev | 46473.21 | 10.00 |
| | Median | 14663.59 | 0.58 |
| | Min | 2800.00 | 0 |
| | Max | 842881.60 | 120 |
| (5) | N | 53 | 50 |
| | Mean | 33987.52 | 3.89 |
| | Std Dev | 41641.28 | 7.85 |
| | Median | 23488.50 | 0.89 |
| | Min | 2800.00 | 0 |
| | Max | 241140.20 | 46.37 |
| (6) | N | 4 | 4 |
| | Mean | 27054.11 | 5.64 |
| | Std Dev | 17779.68 | 11.28 |
| | Median | 20388.63 | 0 |
| | Min | 14414.02 | 0 |
| | Max | 53025.15 | 22.56 |
| (7) | N | 428 | 412 |
| | Mean | 23630.45 | 3.16 |
| | Std Dev | 46335.75 | 9.76 |
| | Median | 14957.30 | 0.63 |
| | Min | 2800.00 | 0 |
| | Max | 842881.60 | 120 |
| (8) | N | 11 | 11 |
| | Mean | 27375.59 | 5.44 |
| | Std Dev | 18719.05 | 10.38 |
| | Median | 20600.96 | 0 |
| | Min | 2800.00 | 0 |
| | Max | 62392.64 | 28.87 |
| (9) | N | 34 | 32 |
| | Mean | 30438.72 | 2.39 |
| | Std Dev | 34101.17 | 4.62 |
| | Median | 19665.28 | 0.86 |
| | Min | 2800.00 | 0 |
| | Max | 188961.30 | 21.22 |
| (10) | N | 23 | 22 |
| | Mean | 38027.77 | 6.39 |
| | Std Dev | 48677.71 | 11.01 |
| | Median | 23940.18 | 2.00 |
| | Min | 2800.00 | 0 |
| | Max | 241140.20 | 46.37 |
| (11) | N | 405 | 390 |
| | Mean | 22873.30 | 3.03 |
| | Std Dev | 46142.56 | 9.71 |
| | Median | 14716.68 | 0.61 |
| | Min | 2800.00 | 0 |
| | Max | 842881.60 | 120 |
| (12) | N | 34 | 33 |
| | Mean | 33861.11 | 5.48 |
| | Std Dev | 41365.57 | 10.24 |
| | Median | 23822.60 | 0.69 |
| | Min | 2800.00 | 0 |
| | Max | 241140.20 | 46.37 |

The following Tables 8 and 9 present results obtained by analyzing the univariate ability of each of the individual marker assays to distinguish between various subject groups. Data are presented as area under the ROC curve for the particular group comparison, calculated according to standard receiver operator characteristic methods, and include p values calculated using a Kruskal-Wallis Test assessing whether two groups are statistically different.

TABLE 8

| | Group (1) v | Group (1) v | Group (4) v |
|---|---|---|---|
| | Group (2) | Group (3) | Group (2) |
| | ROC Area (p | ROC Area (p | ROC Area (p |
| | value for | value for | value for |
| Marker | comparison) | comparison) | comparison) |
| Acidic Calponin | 0.73 (<0.0001) | 0.75 (<0.0001) | 0.52 (0.56) |
| Adrenomedullin | 0.75 (<0.0001) | 0.80 (<0.0001) | 0.58 (0.056) |
| Angiopoietin-4 | 0.52 (0.79) | 0.52 (0.7328) | 0.53 (0.53) |
| Basic Calponin | 0.90 (<0.0001) | 0.92 (<0.0001) | 0.54 (0.29) |
| BNP | 0.65 (0.011) | 0.66 (0.011) | 0.62 (0.008) |
| BMP-4 | 0.58 (0.15) | 0.64 (0.017) | 0.52 (0.70) |
| BNP 1-108 | 0.74 (<0.0001) | 0.75 (<0.0001) | 0.59 (0.020) |
| BNP 3-108 | 0.69 (0.0007) | 0.70 (0.0005) | 0.60 (0.01) |
| BNP 79-108 | 0.67 (0.0003) | 0.68 (0.0002) | 0.60 (0.0025) |
| CCL11 | 0.66 (0.0058) | 0.66 (0.0064) | 0.58 (0.048) |
| CGRP | 0.55 (0.37) | 0.57 (0.22) | 0.54 (0.29) |
| CK-BB | 0.74 (<0.0001) | 0.76 (<0.0001) | 0.53 (0.50) |
| CK-MB | 0.51 (0.85) | 0.51 (0.88) | 0.59 (0.029) |
| CRP | 0.78 (<0.0001) | 0.74 (<0.0001) | 0.79 (<0.0001) |
| D-Dimer | 0.96 (<0.0001) | 0.97 (<0.0001) | 0.89 (<0.0001) |
| sELAF | 0.75 (<0.0001) | 0.79 (<0.0001) | 0.60 (0.011) |
| Endothelin-1 | 0.67 (0.0028) | 0.67 (0.0041) | 0.57 (0.11) |
| GSTP | 0.79 (<0.0001) | 0.81 (<0.0001) | 0.63 (0.0021) |
| hFABP | 0.51 (0.88) | 0.58 (0.19) | 0.52 (0.59) |
| IL-1ra | 0.78 (<0.0001) | 0.78 (<0.0001) | 0.54 (0.29) |
| IL-25 | 0.60 (0.10) | 0.60 (0.10) | 0.56 (0.20) |
| Leptin | 0.65 (0.03) | 0.65 (0.025) | 0.56 (0.28) |
| sLTBR | 0.66 (0.006) | 0.67 (0.0041) | 0.64 (0.0006) |
| MCP-1 | 0.57 (0.27) | 0.51 (0.85) | 0.51 (0.81) |
| MMP9 | 0.94 (<0.0001) | 0.93 (<0.0001) | 0.58 (0.19) |
| MPO | 0.75 (<0.0001) | 0.71 (0.0003) | 0.77 (<0.0001) |
| MYO | 0.54 (0.52) | 0.51 (0.91) | 0.51 (0.85) |
| NDKA | 0.88 (<0.0001) | 0.87 (<0.0001) | 0.67 (<0.0001) |
| Neuropilin-2 | 0.80 (<0.0001) | 0.78 (<0.0001) | 0.52 (0.72) |
| NGAL | 0.75 (0.0003) | 0.76 (<0.0001) | 0.50 (0.94) |
| PLGF-1 | 0.60 (0.057) | 0.57 (0.21) | 0.59 (0.032) |
| PLGF-1/2 | 0.75 (<0.0001) | 0.77 (<0.0001) | 0.59 (0.037) |
| Activated | 0.65 (0.0055) | 0.59 (0.09) | 0.57 (0.074) |
| Protein C | | | |
| Total Protein C | 0.83 (<0.0001) | 0.84 (<0.0001) | 0.51 (0.73) |
| Latent + Active | | | |
| PSAP-A | 0.53 (0.61) | 0.52 (0.73) | 0.56 (0.14) |
| PSAP-A | 0.65 (0.0066) | 0.64 (0.013) | 0.59 (0.025) |
| PSAP-B | 0.52 (0.67) | 0.53 (0.56) | 0.58 (0.044) |
| PSAP-C | 0.62 (0.017) | 0.60 (0.049) | 0.53 (0.35) |
| PSAP-D | 0.52 (0.72) | 0.51 (0.85) | 0.52 (0.62) |
| sRAGE | 0.63 (0.017) | 0.63 (0.027) | 0.52 (0.58) |
| sPECAM-1 | 0.62 (0.090) | 0.68 (0.0076) | 0.51 (0.86) |
| Spectrin 120 | 0.56 (0.32) | 0.56 (0.31) | 0.55 (0.36) |
| Spectrin 145 | 0.52 (0.71) | 0.55 (0.25) | 0.50 (0.99) |
| TIE-2 | 0.62 (0.03) | 0.65 (0.0082) | 0.50 (0.91) |
| Tissue Factor | 0.68 (0.0017) | 0.68 (0.0019) | 0.58 (0.057) |
| sTNF-R1a | 0.79 (<0.0001) | 0.76 (<0.0001) | 0.61 (0.0079) |
| sTNFRSF7 | 0.57 (0.22) | 0.63 (0.029) | 0.52 (0.55) |
| TNFsR14 | 0.66 (0.0038) | 0.63 (0.022) | 0.60 (0.018) |
| cTNI | 0.55 (0.11) | 0.54 (0.23) | 0.54 (0.020) |
| TpP | 0.60 (0.18) | 0.62 (0.089) | 0.53 (0.65) |
| UFDP1H | 0.83 (<0.0001) | 0.87 (<0.0001) | 0.61 (0.066) |
| UPA | 0.66 (0.0055) | 0.62 (0.046) | 0.51 (0.87) |
| VCAM-1 | 0.70 (0.0005) | 0.75 (<0.0001) | 0.53 (0.55) |
| VE Cadherin | 0.61 (0.068) | 0.56 (0.36) | 0.57 (0.091) |
| VEGF | 0.69 (0.0006) | 0.70 (0.0004) | 0.50 (0.95) |
| sVEGF-r1 | 0.73 (0.0001) | 0.72 (0.0005) | 0.66 (0.0003) |
| sVEGF-r2 | 0.59 (0.12) | 0.52 (0.74) | 0.51 (0.77) |
| VWF-Integrin | 0.71 (0.0002) | 0.67 (0.0029) | 0.62 (0.0026) |
| ANP₂₈₋₁₅₁ | 0.61 (0.046) | 0.57 (0.20) | 0.57 (0.13) |

Preferred assays for diagnosis of VETD, PE, and/or DVT include those configured to detect one or more markers providing a ROC area of ≧0.6 in any of the three comparison groups in the preceeding table. These include one or more assays detecting one or more of the following markers: acidic calponin, adrenomedullin, basic calponin, BMP-4, BNP, BNP₁₋₁₀₈, BNP₃₋₁₀₈, BNP₇₉₋₁₀₈, CCL11, CK-BB, CRP, D-dimer, sELAF, endothelin-1, GSTP, IL-1ra, IL-25, leptin, sLTBR, MMP-9, MPO, NDKA, neuropilin-2, NGAL, PLGF-1, PLGF 1+2, activated protein C, total protein C, PSAP-A, PSAP-C, sRAGE, sPECAM-1, TIE-2, tissue factor, sTNFR1a, sTNFRSF7, TNFsR14, TpP, UFDP1H, UPA, VCAM-1, VE-cadherin, VEGF, sVEGF-R1, VWF-integrin, and ANP₂₈₋₁₅₁. Particularly preferred assays include those detecting markers providing a ROC area of ≧0.7 in any of the three comparison groups in the preceeding table. These include one or more assays detecting one or more of the following markers: acidic calponin, adrenomedullin, basic calponin, BNP₁₋₁₀₈, BNP₃₋₁₀₈, CK-BB, CRP, D-dimer, sELAF, GSTP, IL-1ra, MMP-9, MPO, NDKA, neuropilin-2, NGAL, PLGF 1+2, total protein C, sTNFR1a, UFDP1H, VCAM-1, VEGF, sVEGF-R1, and VWF-integrin. Most preferred assays include those detecting markers providing a ROC area of ≧0.8 in any of the three comparison groups in the preceeding table. These include one or more assays detecting one or more of the following markers: adrenomedullin, basic calponin, D-dimer, GSTP, MMP-9, NDKA, neuropilin-2, total protein C, and UFDP1H.

Certain assays, such as those detecting one or more of BNP, BNP₃₋₁₀₈, BNP₇₉₋₁₀₈, CRP, D-dimer, sELAF, GSTP, sLTBR, MPO, NDKA, sTNFR1a, TNFsR14, UFDP1H, sVEGF-R1, and VWF-integrin, which provide a ROC area of ≧0.6 in distinguishing group (4) from group (2), are preferred for distinguishing amongst causes of VETD, and particularly in distinguishing PE from DVT.

Each of these preferred markers and marker assays may be used individually, or in panels comprising 1, 2, 3, 4, 5, 6, or more of these preferred markers, and optionally comprising one or more additional markers other than these preferred markers. The diagnostic methods of the present invention may be used to rule in or out VETD, PE, and/or DVT, most preferably when used together with other clinical signs and symptoms related to these conditions.

TABLE 9

| | Group (5) v | Group (7) v | Group (9) v | Group (11) v |
|---|---|---|---|---|
| | Group (6) | Group (8) | Group (10) | Group (12) |
| | ROC Area (p value | ROC Area (p value | ROC Area (p value | ROC Area (p value |
| Marker | for comparison) | for comparison) | for comparison) | for comparison) |
| Acidic Calponin | 0.76 (0.08) | 0.68 (0.042) | 0.51 (0.93) | 0.57 (0.19) |
| Adrenomedullin | 0.71 (0.16) | 0.67 (0.049) | 0.56 (0.47) | 0.64 (0.0062) |
| Angiopoietin-4 | 0.60 (0.51) | 0.59 (0.30) | 0.53 (0.73) | 0.50 (0.93) |
| Basic Calponin | 0.72 (0.15) | 0.66 (0.07) | 0.60 (0.18) | 0.64 (0.0071) |
| BNP | 0.67 (0.32) | 0.72 (0.019) | 0.60 (0.22) | 0.71 (<0.0001) |
| BMP-4 | 0.70 (0.18) | 0.70 (0.026) | 0.56 (0.42) | 0.61 (0.037) |
| BNP 1-108 | 0.63 (0.39) | 0.68 (0.031) | 0.62 (0.11) | 0.69 (<0.0001) |
| BNP 3-108 | 0.56 (0.69) | 0.69 (0.026) | 0.65 (0.063) | 0.73 (<0.0001) |
| BNP 79-108 | 0.59 (0.50) | 0.62 (0.096) | 0.61 (0.11) | 0.66 (0.0001) |
| CCL11 | 0.56 (0.68) | 0.58 (0.35) | 0.68 (0.019) | 0.65 (0.0034) |
| CGRP | 0.59 (0.55) | 0.56 (0.48) | 0.61 (0.16) | 0.57 (0.15) |
| CK-BB | 0.72 (0.14) | 0.74 (0.0057) | 0.64 (0.078) | 0.65 (0.0027) |
| CK-MB | 0.71 (0.17) | 0.62 (0.16) | 0.58 (0.30) | 0.58 (0.10) |
| CRP | 0.80 (0.046) | 0.73 (0.010) | 0.61 (0.16) | 0.77 (<0.0001) |
| D-Dimer | 0.63 (0.46) | 0.70 (0.035) | 0.63 (0.10) | 0.82 (<0.0001) |
| sELAF | 0.74 (0.12) | 0.61 (0.20) | 0.62 (0.13) | 0.65 (0.0039) |
| Endothelin-1 | 0.51 (0.92) | 0.52 (0.82) | 0.58 (0.32) | 0.58 (0.14) |
| GSTP | 0.58 (0.59) | 0.56 (0.50) | 0.52 (0.78) | 0.58 (0.12) |
| hFABP | 0.67 (0.26) | 0.66 (0.079) | 0.51 (0.86) | 0.55 (0.33) |
| IL-1ra | 0.63 (0.40) | 0.63 (0.14) | 0.60 (0.20) | 0.64 (0.0076) |
| IL-25 | 0.81 (0.043) | 0.60 (0.28) | 0.56 (0.46) | 0.51 (0.92) |
| Leptin | 0.91 (0.058) | 0.74 (0.097) | 0.53 (0.79) | 0.57 (0.34) |
| sLTBR | 0.64 (0.35) | 0.61 (0.20) | 0.54 (0.59) | 0.65 (0.0037) |
| MCP-1 | 0.81 (0.14) | 0.64 (0.33) | 0.54 (0.72) | 0.52 (0.80) |
| MMP9 | 0.71 (0.33) | 0.73 (0.11) | 0.57 (0.56) | 0.68 (0.016) |
| MPO | 0.75 (0.092) | 0.66 (0.064) | 0.63 (0.096) | 0.74 (<0.0001) |
| MYO | 0.56 (0.72) | 0.56 (0.51) | 0.58 (0.34) | 0.52 (0.75) |
| NDKA | 0.74 (0.11) | 0.65 (0.097) | 0.57 (0.35) | 0.52 (0.70) |
| Neuropilin-2 | 0.57 (0.67) | 0.63 (0.19) | 0.55 (0.57) | 0.57 (0.21) |
| NGAL | 0.74 (0.26) | 0.51 (0.95) | 0.54 (0.72) | 0.51 (0.93) |
| PLGF-1 | 0.55 (0.73) | 0.51 (0.93) | 0.57 (0.38) | 0.57 (0.16) |
| PLGF-1/2 | 0.61 (0.45) | 0.53 (0.76) | 0.57 (0.36) | 0.58 (0.11) |
| Activated | 0.56 (0.69) | 0.59 (0.29) | 0.64 (0.073) | 0.60 (0.041) |
| Protein C | | | | |
| Total Protein C | 0.57 (0.64) | 0.55 (0.59) | 0.52 (0.76) | 0.52 (0.66) |
| Latent + Active | | | | |
| PSAP-A | 0.86 (0.016) | 0.69 (0.030) | 0.56 (0.42) | 0.61 (0.028) |
| PSAP-A | 0.66 (0.30) | 0.54 (0.63) | 0.61 (0.15) | 0.60 (0.050) |
| PSAP-B | 0.81 (0.039) | 0.62 (0.19) | 0.57 (0.41) | 0.59 (0.076) |
| PSAP-C | 0.59 (0.52) | 0.56 (0.47) | 0.63 (0.081) | 0.57 (0.12) |
| PSAP-D | 0.67 (0.26) | 0.58 (0.34) | 0.55 (0.50) | 0.53 (0.51) |
| sRAGE | 0.67 (0.27) | 0.59 (0.30) | 0.56 (0.43) | 0.51 (0.84) |
| sPECAM-1 | 0.74 (0.27) | 0.81 (0.033) | 0.52 (0.87) | 0.61 (0.15) |
| Spectrin 120 | 0.56 (0.78) | 0.51 (0.96) | 0.59 (0.36) | 0.58 (0.23) |
| Spectrin 145 | 0.69 (0.19) | 0.65 (0.13) | 0.51 (0.86) | 0.53 (0.52) |
| TIE-2 | 0.56 (0.68) | 0.51 (0.94) | 0.52 (0.78) | 0.51 (0.90) |
| Tissue Factor | 0.50 (0.99) | 0.51 (0.87) | 0.62 (0.13) | 0.60 (0.059) |
| sTNF-R1a | 0.69 (0.21) | 0.69 (0.031) | 0.56 (0.42) | 0.67 (0.0013) |
| sTNFRSF7 | 0.75 (0.098) | 0.65 (0.096) | 0.57 (0.38) | 0.60 (0.057) |
| TNFsR14 | 0.63 (0.41) | 0.56 (0.51) | 0.65 (0.056) | 0.62 (0.017) |
| cTNI | 0.58 (0.42) | 0.52 (0.69) | 0.59 (0.079) | 0.57 (0.0023) |
| TpP | 0.64 (0.53) | 0.54 (0.82) | 0.52 (0.86) | 0.51 (0.87) |
| UFDP1H | 0.85 (0.10) | 0.53 (0.83) | 0.59 (0.40) | 0.52 (0.74) |
| UPA | 0.79 (0.053) | 0.55 (0.60) | 0.53 (0.67) | 0.53 (0.61) |
| VCAM-1 | 0.58 (0.59) | 0.52 (0.87) | 0.61 (0.18) | 0.51 (0.80) |
| VE Cadherin | 0.63 (0.47) | 0.57 (0.46) | 0.54 (0.64) | 0.56 (0.30) |
| VEGF | 0.80 (0.042) | 0.70 (0.022) | 0.66 (0.050) | 0.59 (0.083) |
| sVEGF-r1 | 0.74 (0.41) | 0.52 (0.88) | 0.67 (0.055) | 0.66 (0.0064) |
| sVEGF-r2 | 0.58 (0.57) | 0.53 (0.74) | 0.57 (0.39) | 0.56 (0.26) |
| sVWF-Integrin | 0.53 (0.83) | 0.63 (0.13) | 0.55 (0.52) | 0.64 (0.0087) |
| ANP₂₈₋₁₅₁ | 0.62 (0.41) | 0.55 (0.59) | 0.61 (0.16) | 0.56 (0.21) |

Preferred assays for prognosis of subjects diagnosed VETD, PE, and/or DVT include those configured to detect one or more markers providing a ROC area of ≧0.6 in any of the four comparison groups in the preceeding table. These include one or more assays detecting one or more of the following markers: acidic calponin, adrenomedullin, angiopoietin-4, basic calponin, BMP-4, BNP, BNP₁₋₁₀₈, BNP₃₋₁₀₈, BNP₇₉₋₁₀₈, CCL11, CGRP, CK-BB, CK-MB, CRP, D-dimer, sELAF, hFABP, IL-1ra, IL-25, leptin, sLTBR, MCP-1, MMP-9, MPO, NDKA, neuropilin-2, NGAL, PLGF 1+2, activated protein C, PSAP-A, PSAP-B, PSAP-C, PSAP-D, sRAGE, sPECAM-1, spectrin 145, tissue factor, sTNFR1a, sTNFRSF7, TNFsR14, TpP, UFDP1H, UPA, VCAM-1, VE-cadherin, VEGF, sVEGF-R1, VWF-integrin, and ANP₂₈₋₁₅₁. Particularly preferred assays include those detecting markers providing a ROC area of ≧0.7 in any of the four comparison groups in the preceeding table. These include one or more assays detecting one or more of the following markers: acidic calponin, adrenomedullin, basic calponin, BMP-4, BNP, BNP₃₋₁₀₈, CK-BB, CK-MB, CRP, D-dimer, sELAF, IL-25, leptin, MCP-1, MMP-9, MPO, NDKA, NGAL, PSAP-A, PSAP-B, sPECAM-1, sTNFRSF7, UFDP1H, UPA, VEGF, and sVEGF-R1. Most preferred assays include those detecting markers providing a ROC area of ≧0.8 in any of the four comparison groups in the preceeding table. These include one or more assays detecting one or more of the following markers: CRP, D-dimer, IL-25, leptin, MCP-1, PSAP-A, PSAP-B, sPECAM-1, UFDP1H, and VEGF.

Each of these preferred markers and marker assays may be used individually, or in panels comprising 1, 2, 3, 4, 5, 6, or more of these preferred markers, and optionally comprising one or more additional markers other than these preferred markers. The prognostic methods of the present invention may be used to assign a prognosis to a subject diagnosed as having VETD, PE, and/or DVT, most preferably when used together with other clinical signs and symptoms related to these conditions.

### Example 12

### Panels for the Diagnosis of Pulmonary Embolism

In the following example, the “diseased” dataset represents a population of subjects from the subject collection described in Example 10 diagnosed as having PE and an adverse outcome as defined above in Example 3, while the “normal” dataset represents a population of subjects from the subject collection described in Example 10 for which a PE diagnosis was excluded. Samples were obtained for these subjects at presentation. Panels of markers were identified using the methods described in PCT application no. US03/41426, filed Dec. 23, 2003, and Example 8.

The following markers were selected for use in panels: D-Dimer (univariate ROC 0.887); MPO (univariate ROC 0.789); NDKA (univariate ROC 0.682); Neuropilin-2 (univariate ROC 0.52); CRP (univariate ROC 0.809); VEGF-r1 (univariate ROC 0.651); TNF-sR14 (univariate ROC 0.614); sPECAM-1 (univariate ROC 0.51); Tissue Factor (univariate ROC 0.588).

Panel #

| | 1 | 2 |
|---|---|---|
| Markers in panel | D-dimer, MPO, | D-dimer, MPO, |
| | NDKA, CRP, | NDKA, |
| | VEGF-R1 | TNFsR14, |
| | | Tissue Factor |
| “Normal” n | 219 | 269 |
| “Disease” n | 45 | 51 |
| Ave ROC Area | 0.949 | 0.954 |
| SD(%) | 0.004 | 0.003 |
| Ave Sens @ 92.5% | 79% | 80% |
| Spec | | |
| SD(%) | 3.1 | 3.5 |
| Ave Spec @ 92.5% | 81% | 83% |
| Sens | | |
| SD(%) | 3.1 | 2.3 |

Panel #

| | 3 | 4 |
|---|---|---|
| Markers in panel | Derived marker: | Derived marker: |
| | (D-dimer)/ | (D-dimer)/ |
| | (VCAM-1) | (NDKA) |
| “Normal” n | 261 | 270 |
| “Disease” n | 47 | 51 |
| ROC Area | 0.91 | 0.91 |

| | Panel # | | |
|---|---|---|---|
| | 5 | 6 | 7 |
| Markers in | Derived marker: | Derived marker: | Derived marker: |
| panel | (D-dimer) × | (D-dimer)/ | (D-dimer)/ |
| | (VCAM-1) | (Neuoropilin-2) | (sPECAM-1) |
| “Normal” n | 261 | 253 | 63 |
| “Disease” n | 47 | 48 | 22 |
| Ave ROC Area | 0.90 | 0.90 | 0.90 |

As is demonstrated by the foregoing data, panels comprising assays that detect the preferred markers from Example 11 can be used to assign a diagnosis of pulmonary embolism to a subject. Moreover, as measured by ROC area, such panels can provide diagnostic methods superior to methods in which a marker is used individually. Such panels preferably comprise 2, 3, 4, 5, 6, 7, 8, 9, 10, or more of the preferred marker assays from Example 11. Most preferred panels comprise an assay that detects D-dimer, and one or more assays that detect one or more of the preferred markers from Example 11 other than D-dimer.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method for assigning a diagnosis to a subject suspected of having venous thromboembolic disease, or a prognosis to a subject diagnosed with venous thromboembolic disease, comprising:
performing an assay method on a sample obtained from said subject, wherein said assay method provides a plurality of detectable signals related to the presence or amount of a plurality of subject-derived markers independently selected from the group consisting of markers related to blood pressure regulation, markers related to inflammation, markers related to apoptosis, markers related to reactive oxygen species, markers related to myocardial injury, markers related to pulmonary injury, and markers related to coagulation and hemostasis; and
correlating the signals obtained from said assay method to the presence or absence of venous thromboembolic disease in said subject, or to a likelihood of an outcome in said subject.

2. A method according to claim 1, wherein the correlating step comprises determining the concentration of each of said plurality of subject-derived markers, and individually comparing each marker concentration to a threshold level that is indicative of the presence or absence of venous thromboembolic disease in said subject, or to the likelihood of an outcome in said subject.

3. A method according to claim 1, wherein the correlating step comprises determining the concentration of each of said plurality of subject-derived markers, calculating a single panel response value based on the concentration of each of said plurality of subject-derived markers, and comparing the index value to a threshold level that is indicative of the presence or absence of venous thromboembolic disease in said subject, or to a likelihood of an outcome in said subject.

4. A method according to claim 1, wherein the plurality of markers comprises at least one marker related to coagulation and hemostasis and at least one marker related to inflammation.

5. A method according to claim 1, wherein the plurality of markers comprises at least one marker related to blood pressure regulation and at least one marker related to coagulation and hemostasis.

6. A method according to claim 1, wherein the plurality of markers comprises at least one marker related to pulmonary injury and at least one marker related to blood pressure regulation.

7. A method according to claim 1, wherein the plurality of markers comprises at least one marker related to blood pressure regulation, at least one marker related to inflammation, and at least one marker related to coagulation and hemostasis.

8. A method according to claim 1, wherein the plurality of markers comprises at least one marker related to apoptosis and at least one marker related to coagulation and hemostasis.

9. A method according to claim 1, wherein the plurality of markers comprises at least one marker related to reactive oxygen species and at least one marker related to coagulation and hemostasis.

10. A method according to claim 1, wherein the plurality of markers comprises at least one marker related to myocardial injury and at least one marker related to coagulation and hemostasis.

11. A method according to claim 1, wherein the plurality of markers comprises at least one marker related to pulmonary injury and at least one marker related to coagulation and hemostasis.

12. A method according to claim 1, wherein the plurality of markers comprises at least one marker related to blood pressure regulation, at least one marker related to myocardial injury, and at least one marker related to coagulation and hemostasis.

13. A method according to claim 1, wherein the plurality of markers comprises at least one marker related to blood pressure regulation, at least one marker related to pulmonary injury, and at least one marker related to coagulation and hemostasis.

14. A method according to claim 1, wherein the plurality of markers comprises at least one marker related to blood pressure regulation, at least one marker related to apoptosis, and at least one marker related to coagulation and hemostasis.

15. A method according to claim 1, wherein the plurality of markers comprises at least one marker related to blood pressure regulation, at least one marker related to apoptosis, at least one marker related to inflammation, and at least one marker related to coagulation and hemostasis.

16. A method according to claim 1, wherein or the plurality of markers comprises at least one marker related to blood pressure regulation, at least one marker related to myocardial injury, at least one marker related to pulmonary injury, and at least one marker related to coagulation and hemostasis.

17. A method according to claim 1, wherein the sample is from a human.

18. A method according to claim 1, wherein the sample is selected from the group consisting of blood, serum, and plasma.

19. A method according to claim 1, wherein the assay method is an immunoassay method.

20. A method according to claim 1, wherein the plurality of subject-derived markers comprise one or more markers related to blood pressure regulation selected from the group consisting of atrial natriuretic factor, B-type natriuretic peptide, a marker related to B-type natriuretic peptide, C-type natriuretic peptide, urotensin II, urocortin I, urocortin II, urocortin III, arginine vasopressin, aldosterone, angiotensin I, angiotensin II, angiotensin III, bradykinin, calcitonin, procalcitonin, calcitonin gene related peptide, adrenomedullin, calcyphosine, endothelin-2, endothelin-3, renin, and urodilatin, or marker(s) related thereto.

21. A method according to claim 20, wherein the plurality of subject-derived markers comprise B-type natriuretic peptide, NT-proBNP, proBNP, BNP₇₉₋₁₀₈, or BNP₃₋₁₀₈.

22. A method according to claim 1, wherein the plurality of subject-derived markers comprise one or more markers related to inflammation selected from the group consisting of acute phase reactants, vascular cell adhesion molecule, intercellular adhesion molecule-1, intercellular adhesion molecule-2, intercellular adhesion molecule-3, C-reactive protein, caspase-1, HMG-1, IL-1β, IL-6, IL-8, interleukin-1 receptor agonist, monocyte chemotactic protein-1, caspase-3, lipocalin-type prostaglandin D synthase, mast cell tryptase, eosinophil cationic protein, KL-6, haptoglobin, tumor necrosis factor α, tumor necrosis factor β, fibronectin, macrophage migration inhibitory factor, and vascular endothelial growth factor, or marker(s) related thereto.

23. A method according to claim 22, wherein the plurality of subject-derived markers comprise one or more acute phase reactants selected from the group consisting of hepcidin, HSP-60, HSP-65, HSP-70, S-FAS ligand, asymmetric dimethylarginine, matrix metalloproteins 11, 3, and 9, defensin HBD 1, defensin HBD 2, serum amyloid A, oxidized LDL, insulin like growth factor, transforming growth factor β, an inter-α-inhibitor, e-selectin, glutathione-S-transferase, hypoxia-inducible factor-1α, inducible nitric oxide synthase, intracellular adhesion molecule, lactate dehydrogenase, monocyte chemoattractant peptide-1, n-acetyl aspartate, prostaglandin E2, receptor activator of nuclear factor ligand, TNF receptor superfamily member 1A, and cystatin C, or marker(s) related thereto.

24. A method according to claim 1, wherein the plurality of subject-derived markers comprise one or more markers related to coagulation and hemostasis selected from the group consisting of plasmin, fibrinogen, D-dimer, β-thromboglobulin, platelet factor 4, fibrinopeptide A, platelet-derived growth factor, prothrombin fragment 1+2, plasmin-α2-antiplasmin complex, thrombin-antithrombin III complex, P-selectin, thrombin, von Willebrand factor, tissue factor, and thrombus precursor protein, or marker(s) related thereto.

25. A method according to claim 1, wherein the plurality of subject-derived markers comprise one or more markers related to apoptosis selected from the group consisting of s-acetyl glutathione, cytochrome C, caspase 3, cathepsin D, and α-spectrin, or marker(s) related thereto.

26. A method according to claim 1, wherein the plurality of subject-derived markers comprise one or more markers related to myocardial injury selected from the group consisting of free cardiac troponin I, complexed cardiac troponin I, free and complexed cardiac troponin I, free cardiac troponin T, complexed cardiac troponin T, free and complexed cardiac troponin T, total cardiac troponin, annexin V, B-enolase, CK-MB, glycogen phosphorylase-BB, heart type fatty acid binding protein, phosphoglyceric acid mutase, and S-100ao, or marker(s) related thereto.

27. A method according to claim 1, wherein the plurality of subject-derived markers comprise one or more markers related to pulmonary injury selected from the group consisting of neutrophil elastase, KL-6, LAMP 3, LAMP3, pulmonary surfactant protein A, pulmonary surfactant protein B, pulmonary surfactant protein C, pulmonary surfactant protein D, phospholipase D, PLA2G5, SFTPC, HT156, and HTI1280, or markers related thereto

28. A method according to claim 1, wherein the plurality of subject-derived markers comprise one or more markers related to reactive oxygen species selected from the group consisting of superoxide dismutase, glutathione, ce-tocopherol, ascorbate, inducible nitric oxide synthase, lipid peroxidation products, nitric oxide, myeloperoxidase, and breath hydrocarbons (preferably ethane), or markers related thereto.

29. A method according to claim 1, wherein the plurality of subject-derived markers comprise one or more markers selected from the group consisting of B-type natriuretic peptide, NT-proBNP, proBNP, BNP₇₉₋₁₀₈, BNP₃₋₁₀₈, caspase-3, CRP, D-dimer, TpP, MCP-1, MMP-9, myeloperoxidase, free cardiac troponin I, complexed cardiac troponin I, free and complexed cardiac troponin I, free cardiac troponin T, complexed cardiac troponin-T, free and complexed cardiac troponin T, total cardiac troponin, pulmonary surfactant protein A, pulmonary surfactant protein B, pulmonary surfactant protein C, pulmonary surfactant protein D, or marker(s) related thereto.

30. A method according to claim 29, wherein the plurality of subject-derived markers comprise a plurality of markers selected from the group consisting of B-type natriuretic peptide, NT-proBNP, proBNP, BNP₇₉₋₁₀₈, BNP₃₋₁₀₈, caspase-3, CRP, D-dimer, TpP, MCP-1, MMP-9, myeloperoxidase, free cardiac troponin I, complexed cardiac troponin I, free and complexed cardiac troponin I, free cardiac troponin T, complexed cardiac troponin T, free and complexed cardiac troponin T, total cardiac troponin, pulmonary surfactant protein A, pulmonary surfactant protein B, pulmonary surfactant protein C, pulmonary surfactant protein D, or marker(s) related thereto.

31. A method according to claim 29, wherein the plurality of subject-derived markers are selected from the group consisting of B-type natriuretic peptide, NT-proBNP, proBNP, BNP₇₉₋₁₀₈, BNP₃₋₁₀₈, caspase-3, CRP, D-dimer, TpP, MCP-1, MMP-9, myeloperoxidase, free cardiac troponin I, complexed cardiac troponin I, free and complexed cardiac troponin I, free cardiac troponin T, complexed cardiac troponin T, free and complexed cardiac troponin T, total cardiac troponin, pulmonary surfactant protein A, pulmonary surfactant protein B, pulmonary surfactant protein C, pulmonary surfactant protein D, or marker(s) related thereto.

32. A method according to claim 1, further comprising measuring at least one non subject-derived marker, wherein said on subject-derived marker(s) is/are correlated with the presence or absence of venous thromboembolic disease in said subject, or the likelihood of an outcome in said subject.

33. A method according to claim 1, wherein the plurality of subject-derived markers comprise D-dimer, one or more markers related to blood pressure regulation selected from the group consisting of B-type natriuretic peptide, NT-proBNP, proBNP, or BNP₃₋₁₀₈, and one or more markers related to myocardial injury selected from the group consisting of free cardiac troponin I, complexed cardiac troponin I, free and complexed cardiac troponin I, free cardiac troponin T, complexed cardiac troponin T, free and complexed cardiac troponin T, total cardiac troponin, annexin V, B-enolase, CK-MB, glycogen phosphorylase-BB, heart type fatty acid binding protein, phosphoglyceric acid mutase, and S-100ao, pulmonary surfactant protein A, pulmonary surfactant protein B, pulmonary surfactant protein C, pulmonary surfactant protein D, or marker(s) related thereto.

34. A method for assigning a prognosis to a subject diagnosed with venous thromboembolic disease, comprising:
performing an assay method on a sample obtained from said subject, wherein said assay method provides a detectable signal related to the presence or amount of B-type natriuretic peptide or a marker related thereto; and
correlating the signal obtained from said assay method to a likelihood of an outcome in said subject.

35. A method according to claim 34, wherein said assay method further provides one or more detectable signals related to the presence or amount of said one or more other subject-derived markers that are correlated with the likelihood of an outcome in said subject.

36. A method according to claim 35, wherein said one or more other subject-derived markers comprise one or more markers selected from the group consisting of free cardiac troponin I, complexed cardiac troponin I, free and complexed cardiac troponin I, free cardiac troponin T, complexed cardiac troponin T, free and complexed cardiac troponin T, total cardiac troponin, and D-dimer, pulmonary surfactant protein A, pulmonary surfactant protein B, pulmonary surfactant protein C, pulmonary surfactant protein D, or marker(s) related thereto.

37. A method according to claim 34, further comprising measuring at least one characteristic of said subject that is not a subject-derived marker, wherein said characteristic(s) is/are correlated with the likelihood of an outcome in said subject.

38. A method for assigning a prognosis to a subject diagnosed with venous thromboembolic disease, comprising:
performing an assay method on a sample obtained from said subject, wherein said assay method provides a detectable signal related to the presence or amount of one or more markers related to myocardial injury selected from the group consisting of free cardiac troponin I, complexed cardiac troponin I, free and complexed cardiac troponin I, free cardiac troponin T, complexed cardiac troponin T, free and complexed cardiac troponin T, and total cardiac troponin, or a marker related thereto; and
correlating the signal obtained from said assay method to a likelihood of an outcome in said subject.

39. A method according to claim 38, wherein said assay method further provides one or more detectable signals related to the presence or amount of said one or more other subject-derived markers that are correlated with the likelihood of an outcome in said subject.

40. A method according to claim 39, wherein said one or more other subject-derived markers comprise one or more markers selected from the group consisting of B-type natriuretic peptide, NT-proBNP, proBNP, BNP₇₉₋₁₀₈, BNP₃₋₁₀₈, pulmonary surfactant protein A, pulmonary surfactant protein B, pulmonary surfactant protein C, pulmonary surfactant protein D, and D-dimer, or marker(s) related thereto.

41. A method according to claim 38, further comprising measuring at least one characteristic of said subject that is not a subject-derived marker, wherein said characteristic(s) is/are correlated with the likelihood of an outcome in said subject.

42. A method for assigning a diagnosis to a subject suspected of having venous thromboembolic disease, or a prognosis to a subject diagnosed with venous thromboembolic disease, comprising:
performing one or more assays on one or more samples obtained from said subject, wherein said assay(s) are configured to detect one or more markers selected from the group consisting of acidic calponin, adrenomedullin, angiopoietin-4, basic calponin, bone morphogenic protein-4 (BNP-4), B-type natriuretic peptide (BNP), BNP₁₋₁₀₈ (proBNP), BNP₃₋₁₀₈, BNP₇₉₋₁₀₈, CCL11, calcitonin gene-related peptide (CGRP), creatine kinase-BB (CK-BB), creatine kinase-MB (CK-MB), C-reactive protein (CRP), soluble elastin fragment (sELAF), endothelin-1, glutathione-S-transferase 3 (GSTP), heart fatty acid binding protein (hFABP), IL-1ra, IL-25, leptin, soluble lymphotoxin B receptor (sLTBR), monocyte chemotactic protein-1 (MCP-1), matrix metalloproteinase-9 (MMP-9), myeloperoxidase (MPO), nucleotide diphosphate kinase A (NDKA), neuropilin-2, neutrophil gelatinase-associated lipocalin (NGAL), placental growth factor 1 (PLGF-1), placental growth factor 1 and 2 (PLGF 1+2), activated protein C, total protein C, pulmonary surfactant protein A (PSAP-A), pulmonary surfactant protein B (PSAP-B), pulmonary surfactant protein C (PSAP-C), pulmonary surfactant protein D (PSAP-D), soluble receptor for advanced glycosylation end products (sRAGE), soluble platelet endothelial cell adhesion molecule-1 (sPECAM-1), spectrin α-chain 145 kDa (spectrin 145), soluble angiopoietin-1 receptor (TIE-2), tissue factor, soluble tumor necrosis factor receptor 1a (sTNFR1a), soluble tumor necrosis factor receptor superfamily member 7 (sTNFRSF7), soluble tumor necrosis factor receptor superfamily member 14 (TNFsR14), thrombus precursor protein (TpP), ubiquitin fusion degradation protein 1 homolog (UFDP1H), urokinase-type plasminogen activator (UPA), cascular cell adhesion protein 1 (VCAM-1), VE-cadherin, vascular endothelial growth factor (VEGF), soluble flt-1 (sVEGF-R1), von Willebrand factor comprising an integrin domain (VWF-integrin), and ANP₂₈₋₁₅₁; and
relating the results obtained from said assay(s) to the presence or absence of venous thromboembolic disease in said subject, or to a likelihood of an outcome in said subject.

43. A method according to claim 42, wherein said method comprises relating the results obtained from said assay(s) to the presence or absence of pulmonary embolism in said subject.

44. A method according to claim 42, wherein said method comprises relating the results obtained from said assay(s) to the presence or absence of deep vein thrombosis in said subject.

45. A method according to claim 42, wherein said method comprises relating the results obtained from said assay(s) to the distinguishing between pulmonary embolism and deep vein thrombosis in said subject.

46. A method according to claim 42, further comprising performing one or more additional assays configured to detect one or more one or more additional markers in said sample, and said relating step comprises relating the results of said assay(s) and the results of said one or more additional assays to the presence or absence of venous thromboembolic disease in said subject, or to a likelihood of an outcome in said subject.

47. A method according to claim 46, wherein said one or more other markers are independently selected from the group consisting of specific markers of myocardial injury, markers related to pulmonary injury, markers related to blood pressure regulation, markers related to coagulation and hemostasis, markers related to inflammation, and markers related to apoptosis.

48. A method according to claim 46, wherein said one or more additional markers comprise D-dimer.

49. A method according to claim 42, wherein the subject is a human.

50. A method according to claim 42, wherein the sample(s) are selected from the group consisting of blood, serum, and plasma.

51. A method according to claim 42, wherein said one or more assays are one or more immunoassays, and said relating step comprises generating a signal from each of said assay(s) and converting each said signal to a measured concentration of one of said markers.

52. A method according to claim 51, wherein relating step further comprises comparing each said measured concentration to a threshold concentration selected to distinguish a non-diseased population from a diseased population.

53. A method according to claim 42, wherein said method comprises relating the results obtained from said assay(s) to the presence or absence of venous thromboembolic disease in said subject, and said method comprises performing one or more assays on one or more samples obtained from said subject, wherein said assay(s) are configured to detect one or more markers selected from the group consisting of acidic calponin, adrenomedullin, basic calponin, BMP-4, BNP, BNP₁₋₁₀₈, BNP₃₋₁₀₈, BNP₇₉₋₁₀₈, CCL11, CK-BB, CRP, D-dimer, sELAF, endothelin-1, GSTP, IL-1ra, IL-25, leptin, sLTBR, MMP-9, MPO, NDKA, neuropilin-2, NGAL, PLGF-1, PLGF 1+2, activated protein C, total protein C, PSAP-A, PSAP-C, sRAGE, sPECAM-1, TIE-2, tissue factor, sTNFR1a, sTNFRSF7, TNFsR14, TpP, UFDP1H, UPA, VCAM-1, VE-cadherin, VEGF, sVEGF-R1, VWF-integrin, and ANP₂₈₋₁₅₁.

54. A method according to claim 42, wherein said method comprises relating the results obtained from said assay(s) to the presence or absence of venous thromboembolic disease in said subject, and said method comprises performing one or more assays on one or more samples obtained from said subject, wherein said assay(s) are configured to detect one or more markers selected from the group consisting of acidic calponin, adrenomedullin, basic calponin, BNP₁₋₁₀₈, BNP₃₋₁₀₈, CK-BB, CRP, sELAF, GSTP, IL-1ra, MMP-9, MPO, NDKA, neuropilin-2, NGAL, PLGF 1+2, total protein C, sTNFR1a, UFDP1H, VCAM-1, VEGF, sVEGF-R1, and VWF-integrin.

55. A method according to claim 42, wherein said method comprises relating the results obtained from said assay(s) to the presence or absence of venous thromboembolic disease in said subject, and said method comprises performing one or more assays on one or more samples obtained from said subject, wherein said assay(s) are configured to detect one or more markers selected from the group consisting of adrenomedullin, basic calponin, GSTP, MMP-9, NDKA, neuropilin-2, total protein C, and UFDP1H.

56. A method according to claim 42, wherein said method comprises relating the results obtained from said assay(s) to a likelihood of an outcome in said subject, and said method comprises performing one or more assays on one or more samples obtained from said subject, wherein said assay(s) are configured to detect one or more markers selected from the group consisting of acidic calponin, adrenomedullin, angiopoietin-4, basic calponin, BMP-4, BNP, BNP₁₋₁₀₈, BN₃₋₁₀₈, BNP₇₉₋₁₀₈, CCL11, CGRP, CK-BB, CK-MB, CRP, sELAF, hFABP, IL-1ra, IL-25, leptin, sLTBR, MCP-1, MMP-9, MPO, NDKA, neuropilin-2, NGAL, PLGF 1+2, activated protein C, PSAP-A, PSAP-B, PSAP-C, PSAP-D, sRAGE, sPECAM-1, spectrin 145, tissue factor, sTNFR1a, sTNFRSF7, TNFsR14, TpP, UFDP1H, UPA, VCAM-1, VE-cadherin, VEGF, sVEGF-R1, VWF-integrin, and ANP₂₈₋₁₅₁.

57. A method according to claim 42, wherein said method comprises relating the results obtained from said assay(s) to a likelihood of an outcome in said subject, and said method comprises performing one or more assays on one or more samples obtained from said subject, wherein said assay(s) are configured to detect one or more markers selected from the group consisting of acidic calponin, adrenomedullin, basic calponin, BMP-4, BNP, BNP₃₋₁₀₈, CK-BB, CK-MB, CRP, sELAF, IL-25, leptin, MCP-1, MMP-9, MPO, NDKA, NGAL, PSAP-A, PSAP-B, sPECAM-1, sTNFRSF7, UFDP1H, UPA, VEGF, and sVEGF-R1.

58. A method according to claim 42, wherein said method comprises relating the results obtained from said assay(s) to a likelihood of an outcome in said subject, and said method comprises performing one or more assays on one or more samples obtained from said subject, wherein said assay(s) are configured to detect one or more markers selected from the group consisting of CRP, IL-25, leptin, MCP-1, PSAP-A, PSAP-B, sPECAM-1, UFDP1H, and VEGF.

59. A method according to claim 42, wherein said method comprises performing at least 2 said assays.

60. A method according to claim 42, wherein said method comprises performing at least 3 said assays.

61. A method according to claim 42, wherein said method comprises performing at least 4 said assays.

62. A method according to claim 42, wherein said method comprises performing at least 5 said assays.

63. A method according to claim 42, wherein said method comprises performing at least 6 said assays.
